SEQUENCE LISTING (37 sequences)

SEQ ID NO: 1 - DNA, 1080 bp, Artificial Sequence
  DNA coding of expression cassettes constructed for Lym1 single-chain (sL1) expression in Pichia pastoris (aMFsL1XE)
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctagatctcaggtgcagctgaaggagtcaggacctggcctggtggcgccctcacagagcctgtccatcacatgcaccatctcagggttctcattaaccagctatggtgtacactgggttcgccagcctccaggaaagggtctggagtggctggtagtgatatggagtgatggaagcacaacctataattcagctctcaaatccagactgagcatcagcaaggacaactccaagagccaagttttcttaaaaatgaacagtctccaaactgatgacacagccatatactactgtgccagtcactacggtagtacccttgcctttgcttcctggggccacgggactctggtcactgtctctgctggcggtggtggttccggtggtggtggttccggtggtggtggttccgacattcagatgactcagtctccagcctccctatctgcatctgtgggggaaactgtcaccatcatatgtcgagcaagtgtgaatatttacagttatttagcatggtatcagcagaaacagggaaaatctcctcagctcctggtctataatgccaaaatcttagcagaaggtgtgccatcaaggttcagtggcagtggatcaggcacacagttttctctgaagatcaacagcctgcagcctgaagattttgggagttattactgtcaacatcattatggtacattcacgttcggctcggggacaaagttggaaataaaatctagagggcccttcgaaggtaagcctatccctaaccctctcctcggtctcgattctacgcgtaccggtcatcatcaccatcaccattga

SEQ ID NO: 2 - DNA, 1011 bp, Artificial Sequence
  DNA coding of expression cassettes constructed for Lym1 single-chain (sL1) expression in Pichia pastoris (aMFsL1XN)
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctagatctcaggtgcagctgaaggagtcaggacctggcctggtggcgccctcacagagcctgtccatcacatgcaccatctcagggttctcattaaccagctatggtgtacactgggttcgccagcctccaggaaagggtctggagtggctggtagtgatatggagtgatggaagcacaacctataattcagctctcaaatccagactgagcatcagcaaggacaactccaagagccaagttttcttaaaaatgaacagtctccaaactgatgacacagccatatactactgtgccagtcactacggtagtacccttgcctttgcttcctggggccacgggactctggtcactgtctctgctggcggtggtggttccggtggtggtggttccggtggtggtggttccgacattcagatgactcagtctccagcctccctatctgcatctgtgggggaaactgtcaccatcatatgtcgagcaagtgtgaatatttacagttatttagcatggtatcagcagaaacagggaaaatctcctcagctcctggtctataatgccaaaatcttagcagaaggtgtgccatcaaggttcagtggcagtggatcaggcacacagttttctctgaagatcaacagcctgcagcctgaagattttgggagttattactgtcaacatcattatggtacattcacgttcggctcggggacaaagttggaaataaaacaccaccatcaccatcactga

SEQ ID NO: 3 - DNA, 879 bp, Artificial Sequence
  DNA coding of expression cassettes constructed for Lym1 single-chain (sL1) expression in Pichia pastoris (PHO1sL1XE)
atgttctctccaattttgtccttggaaattattttagctttggctactttgcaatctgtcttcgctcgagctcaggtgcagctgaaggagtcaggacctggcctggtggcgccctcacagagcctgtccatcacatgcaccatctcagggttctcattaaccagctatggtgtacactgggttcgccagcctccaggaaagggtctggagtggctggtagtgatatggagtgatggaagcacaacctataattcagctctcaaatccagactgagcatcagcaaggacaactccaagagccaagttttcttaaaaatgaacagtctccaaactgatgacacagccatatactactgtgccagtcactacggtagtacccttgcctttgcttcctggggccacgggactctggtcactgtctctgctggcggtggtggttccggtggtggtggttccggtggtggtggttccgacattcagatgactcagtctccagcctccctatctgcatctgtgggggaaactgtcaccatcatatgtcgagcaagtgtgaatatttacagttatttagcatggtatcagcagaaacagggaaaatctcctcagctcctggtctataatgccaaaatcttagcagaaggtgtgccatcaaggttcagtggcagtggatcaggcacacagttttctctgaagatcaacagcctgcagcctgaagattttgggagttattactgtcaacatcattatggtacattcacgttcggctcggggacaaagttggaaataaaatctagagggcccttcgaaggtaagcctatccctaaccctctcctcggtctcgattctacgcgtaccggtcatcatcaccatcaccattga

SEQ ID NO: 4 - protein, 359 aa, Artificial Sequence
  Translated expression cassette for Lym1 single-chain (sL1) expression in Pichia pastoris (aMFsL1XE)
MRFPSIFTAVLFAASSALAAPVNTTTEDETAQIPAEAVIGYSDLEGDFDVAVLPFSNSTNNGLLFINTTIASIAAKEEGVSLEKREAEARSQVQLKESGPGLVAPSQSLSITCTISGFSLTSYGVHWVRQPPGKGLEWLVVIWSDGSTTYNSALKSRLSISKDNSKSQVFLKMNSLQTDDTAIYYCASHYGSTLAFASWGHGTLVTVSAGGGGSGGGGSGGGGSDIQMTQSPASLSASVGETVTIICRASVNIYSYLAWYQQKQGKSPQLLVYNAKILAEGVPSRFSGSGSGTQFSLKINSLQPEDFGSYYCQHHYGTFTFGSGTKLEIKSRGPFEGKPIPNPLLGLDSTRTGHHHHHH

SEQ ID NO: 5 - protein, 336 aa, Artificial Sequence
  Translated expression cassette for Lym1 single-chain (sL1) expression in Pichia pastoris (aMFsL1XN)
MRFPSIFTAVLFAASSALAAPVNTTTEDETAQIPAEAVIGYSDLEGDFDVAVLPFSNSTNNGLLFINTTIASIAAKEEGVSLEKREAEARSQVQLKESGPGLVAPSQSLSITCTISGFSLTSYGVHWVRQPPGKGLEWLVVIWSDGSTTYNSALKSRLSISKDNSKSQVFLKMNSLQTDDTAIYYCASHYGSTLAFASWGHGTLVTVSAGGGGSGGGGSGGGGSDIQMTQSPASLSASVGETVTIICRASVNIYSYLAWYQQKQGKSPQLLVYNAKILAEGVPSRFSGSGSGTQFSLKINSLQPEDFGSYYCQHHYGTFTFGSGTKLEIKHHHHHH

SEQ ID NO: 6 - protein, 292 aa, Artificial Sequence
  Translated expression cassette for Lym1 single-chain (sL1) expression in Pichia pastoris (PHO1sL1XE)
MFSPILSLEIILALATLQSVFARAQVQLKESGPGLVAPSQSLSITCTISGFSLTSYGVHWVRQPPGKGLEWLVVIWSDGSTTYNSALKSRLSISKDNSKSQVFLKMNSLQTDDTAIYYCASHYGSTLAFASWGHGTLVTVSAGGGGSGGGGSGGGGSDIQMTQSPASLSASVGETVTIICRASVNIYSYLAWYQQKQGKSPQLLVYNAKILAEGVPSRFSGSGSGTQFSLKINSLQPEDFGSYYCQHHYGTFTFGSGTKLEIKSRGPFEGKPIPNPLLGLDSTRTGHHHHHH

SEQ ID NO: 7 - DNA, 1080 bp, Artificial Sequence; identical to SEQ ID NO: 1
  DNA sequence of aMFsL1XE expression cassette

SEQ ID NO: 8 - protein, 359 aa, Artificial Sequence; identical to SEQ ID NO: 4
  Amino acid translation of the aMFsL1XE expression cassette

SEQ ID NO: 9 - protein, 118 aa, Artificial Sequence
  scLym1_VH
QVQLKESGPGLVAPSQSLSITCTISGFSLTSYGVHWVRQPPGKGLEWLVVIWSDGSTTYNSALKSRLSISKDNSKSQVFLKMNSLQTDDTAIYYCASHYGSTLAFASWGHGTLVTVSA

SEQ ID NO: 10 - protein, 5 aa, Artificial Sequence
  VH CDR1
SYGVH

SEQ ID NO: 11 - protein, 16 aa, Artificial Sequence
  VH CDR2
VIWSDGSTTYNSALKS

SEQ ID NO: 12 - protein, 10 aa, Artificial Sequence
  VH CDR3
HYGSTLAFAS

SEQ ID NO: 13 - protein, 15 aa, Artificial Sequence
  (G4S)3_Linker
GGGGSGGGGSGGGGS

SEQ ID NO: 14 - protein, 106 aa, Artificial Sequence
  scLym1_VL
DIQMTQSPASLSASVGETVTIICRASVNIYSYLAWYQQKQGKSPQLLVYNAKILAEGVPSRFSGSGSGTQFSLKINSLQPEDFGSYYCQHHYGTFTFGSGTKLEIK

SEQ ID NO: 15 - protein, 11 aa, Artificial Sequence
  VL CDR1
RASVNIYSYLA

SEQ ID NO: 16 - protein, 7 aa, Artificial Sequence
  VL CDR2
NAKILAE

SEQ ID NO: 17 - protein, 8 aa, Artificial Sequence
  VL CDR3
QHHYGTFT

SEQ ID NO: 18 - DNA, 1080 bp, Artificial Sequence
  p9S31C
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctagatctcaggtgcagctgaaggagtcaggacctggcctggtggcgccctcacagagcctgtccatcacatgcaccatctcagggttctcattaacctgttatggtgtacactgggttcgccagcctccaggaaagggtctggagtggctggtagtgatatggagtgatggaagcacaacctataattcagctctcaaatccagactgagcatcagcaaggacaactccaagagccaagttttcttaaaaatgaacagtctccaaactgatgacacagccatatactactgtgccagtcactacggtagtacccttgcctttgcttcctggggccacgggactctggtcactgtctctgctggcggtggtggttccggtggtggtggttccggtggtggtggttccgacattcagatgactcagtctccagcctccctatctgcatctgtgggggaaactgtcaccatcatatgtcgagcaagtgtgaatatttacagttatttagcatggtatcagcagaaacagggaaaatctcctcagctcctggtctataatgccaaaatcttagcagaaggtgtgccatcaaggttcagtggcagtggatcaggcacacagttttctctgaagatcaacagcctgcagcctgaagattttgggagttattactgtcaacatcattatggtacattcacgttcggctcggggacaaagttggaaataaaatctagagggcccttcgaaggtaagcctatccctaaccctctcctcggtctcgattctacgcgtaccggtcatcatcaccatcaccattga

SEQ ID NO: 19 - DNA, 1080 bp, Artificial Sequence
  p9Y32C
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctagatctcaggtgcagctgaaggagtcaggacctggcctggtggcgccctcacagagcctgtccatcacatgcaccatctcagggttctcattaaccagctgtggtgtacactgggttcgccagcctccaggaaagggtctggagtggctggtagtgatatggagtgatggaagcacaacctataattcagctctcaaatccagactgagcatcagcaaggacaactccaagagccaagttttcttaaaaatgaacagtctccaaactgatgacacagccatatactactgtgccagtcactacggtagtacccttgcctttgcttcctggggccacgggactctggtcactgtctctgctggcggtggtggttccggtggtggtggttccggtggtggtggttccgacattcagatgactcagtctccagcctccctatctgcatctgtgggggaaactgtcaccatcatatgtcgagcaagtgtgaatatttacagttatttagcatggtatcagcagaaacagggaaaatctcctcagctcctggtctataatgccaaaatcttagcagaaggtgtgccatcaaggttcagtggcagtggatcaggcacacagttttctctgaagatcaacagcctgcagcctgaagattttgggagttattactgtcaacatcattatggtacattcacgttcggctcggggacaaagttggaaataaaatctagagggcccttcgaaggtaagcctatccctaaccctctcctcggtctcgattctacgcgtaccggtcatcatcaccatcaccattga

SEQ ID NO: 20 - DNA, 1080 bp, Artificial Sequence
  p9G33C
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctagatctcaggtgcagctgaaggagtcaggacctggcctggtggcgccctcacagagcctgtccatcacatgcaccatctcagggttctcattaaccagctattgtgtacactgggttcgccagcctccaggaaagggtctggagtggctggtagtgatatggagtgatggaagcacaacctataattcagctctcaaatccagactgagcatcagcaaggacaactccaagagccaagttttcttaaaaatgaacagtctccaaactgatgacacagccatatactactgtgccagtcactacggtagtacccttgcctttgcttcctggggccacgggactctggtcactgtctctgctggcggtggtggttccggtggtggtggttccggtggtggtggttccgacattcagatgactcagtctccagcctccctatctgcatctgtgggggaaactgtcaccatcatatgtcgagcaagtgtgaatatttacagttatttagcatggtatcagcagaaacagggaaaatctcctcagctcctggtctataatgccaaaatcttagcagaaggtgtgccatcaaggttcagtggcagtggatcaggcacacagttttctctgaagatcaacagcctgcagcctgaagattttgggagttattactgtcaacatcattatggtacattcacgttcggctcggggacaaagttggaaataaaatctagagggcccttcgaaggtaagcctatccctaaccctctcctcggtctcgattctacgcgtaccggtcatcatcaccatcaccattga

SEQ ID NO: 21 - DNA, 1080 bp, Artificial Sequence
  p9V34C
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctagatctcaggtgcagctgaaggagtcaggacctggcctggtggcgccctcacagagcctgtccatcacatgcaccatctcagggttctcattaaccagctatggttgtcactgggttcgccagcctccaggaaagggtctggagtggctggtagtgatatggagtgatggaagcacaacctataattcagctctcaaatccagactgagcatcagcaaggacaactccaagagccaagttttcttaaaaatgaacagtctccaaactgatgacacagccatatactactgtgccagtcactacggtagtacccttgcctttgcttcctggggccacgggactctggtcactgtctctgctggcggtggtggttccggtggtggtggttccggtggtggtggttccgacattcagatgactcagtctccagcctccctatctgcatctgtgggggaaactgtcaccatcatatgtcgagcaagtgtgaatatttacagttatttagcatggtatcagcagaaacagggaaaatctcctcagctcctggtctataatgccaaaatcttagcagaaggtgtgccatcaaggttcagtggcagtggatcaggcacacagttttctctgaagatcaacagcctgcagcctgaagattttgggagttattactgtcaacatcattatggtacattcacgttcggctcggggacaaagttggaaataaaatctagagggcccttcgaaggtaagcctatccctaaccctctcctcggtctcgattctacgcgtaccggtcatcatcaccatcaccattga

SEQ ID NO: 22 - DNA, 1080 bp, Artificial Sequence
  p9H35C
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctagatctcaggtgcagctgaaggagtcaggacctggcctggtggcgccctcacagagcctgtccatcacatgcaccatctcagggttctcattaaccagctatggtgtatgttgggttcgccagcctccaggaaagggtctggagtggctggtagtgatatggagtgatggaagcacaacctataattcagctctcaaatccagactgagcatcagcaaggacaactccaagagccaagttttcttaaaaatgaacagtctccaaactgatgacacagccatatactactgtgccagtcactacggtagtacccttgcctttgcttcctggggccacgggactctggtcactgtctctgctggcggtggtggttccggtggtggtggttccggtggtggtggttccgacattcagatgactcagtctccagcctccctatctgcatctgtgggggaaactgtcaccatcatatgtcgagcaagtgtgaatatttacagttatttagcatggtatcagcagaaacagggaaaatctcctcagctcctggtctataatgccaaaatcttagcagaaggtgtgccatcaaggttcagtggcagtggatcaggcacacagttttctctgaagatcaacagcctgcagcctgaagattttgggagttattactgtcaacatcattatggtacattcacgttcggctcggggacaaagttggaaataaaatctagagggcccttcgaaggtaagcctatccctaaccctctcctcggtctcgattctacgcgtaccggtcatcatcaccatcaccattga

SEQ ID NO: 23 - protein, 359 aa, Artificial Sequence
  p9S31C
MRFPSIFTAVLFAASSALAAPVNTTTEDETAQIPAEAVIGYSDLEGDFDVAVLPFSNSTNNGLLFINTTIASIAAKEEGVSLEKREAEARSQVQLKESGPGLVAPSQSLSITCTISGFSLTCYGVHWVRQPPGKGLEWLVVIWSDGSTTYNSALKSRLSISKDNSKSQVFLKMNSLQTDDTAIYYCASHYGSTLAFASWGHGTLVTVSAGGGGSGGGGSGGGGSDIQMTQSPASLSASVGETVTIICRASVNIYSYLAWYQQKQGKSPQLLVYNAKILAEGVPSRFSGSGSGTQFSLKINSLQPEDFGSYYCQHHYGTFTFGSGTKLEIKSRGPFEGKPIPNPLLGLDSTRTGHHHHHH

SEQ ID NO: 24 - protein, 359 aa, Artificial Sequence
  p9Y32C
MRFPSIFTAVLFAASSALAAPVNTTTEDETAQIPAEAVIGYSDLEGDFDVAVLPFSNSTNNGLLFINTTIASIAAKEEGVSLEKREAEARSQVQLKESGPGLVAPSQSLSITCTISGFSLTSCGVHWVRQPPGKGLEWLVVIWSDGSTTYNSALKSRLSISKDNSKSQVFLKMNSLQTDDTAIYYCASHYGSTLAFASWGHGTLVTVSAGGGGSGGGGSGGGGSDIQMTQSPASLSASVGETVTIICRASVNIYSYLAWYQQKQGKSPQLLVYNAKILAEGVPSRFSGSGSGTQFSLKINSLQPEDFGSYYCQHHYGTFTFGSGTKLEIKSRGPFEGKPIPNPLLGLDSTRTGHHHHHH

SEQ ID NO: 25 - protein, 359 aa, Artificial Sequence
  p9G33C
MRFPSIFTAVLFAASSALAAPVNTTTEDETAQIPAEAVIGYSDLEGDFDVAVLPFSNSTNNGLLFINTTIASIAAKEEGVSLEKREAEARSQVQLKESGPGLVAPSQSLSITCTISGFSLTSYCVHWVRQPPGKGLEWLVVIWSDGSTTYNSALKSRLSISKDNSKSQVFLKMNSLQTDDTAIYYCASHYGSTLAFASWGHGTLVTVSAGGGGSGGGGSGGGGSDIQMTQSPASLSASVGETVTIICRASVNIYSYLAWYQQKQGKSPQLLVYNAKILAEGVPSRFSGSGSGTQFSLKINSLQPEDFGSYYCQHHYGTFTFGSGTKLEIKSRGPFEGKPIPNPLLGLDSTRTGHHHHHH

SEQ ID NO: 26 - protein, 359 aa, Artificial Sequence
  p9V34C
MRFPSIFTAVLFAASSALAAPVNTTTEDETAQIPAEAVIGYSDLEGDFDVAVLPFSNSTNNGLLFINTTIASIAAKEEGVSLEKREAEARSQVQLKESGPGLVAPSQSLSITCTISGFSLTSYGCHWVRQPPGKGLEWLVVIWSDGSTTYNSALKSRLSISKDNSKSQVFLKMNSLQTDDTAIYYCASHYGSTLAFASWGHGTLVTVSAGGGGSGGGGSGGGGSDIQMTQSPASLSASVGETVTIICRASVNIYSYLAWYQQKQGKSPQLLVYNAKILAEGVPSRFSGSGSGTQFSLKINSLQPEDFGSYYCQHHYGTFTFGSGTKLEIKSRGPFEGKPIPNPLLGLDSTRTGHHHHHH

SEQ ID NO: 27 - protein, 359 aa, Artificial Sequence
  p9H35C
MRFPSIFTAVLFAASSALAAPVNTTTEDETAQIPAEAVIGYSDLEGDFDVAVLPFSNSTNNGLLFINTTIASIAAKEEGVSLEKREAEARSQVQLKESGPGLVAPSQSLSITCTISGFSLTSYGVCWVRQPPGKGLEWLVVIWSDGSTTYNSALKSRLSISKDNSKSQVFLKMNSLQTDDTAIYYCASHYGSTLAFASWGHGTLVTVSAGGGGSGGGGSGGGGSDIQMTQSPASLSASVGETVTIICRASVNIYSYLAWYQQKQGKSPQLLVYNAKILAEGVPSRFSGSGSGTQFSLKINSLQPEDFGSYYCQHHYGTFTFGSGTKLEIKSRGPFEGKPIPNPLLGLDSTRTGHHHHHH

SEQ ID NO: 28 - protein, 10 aa, Artificial Sequence
  VH CDR1 definition under AbM method
GFSLTSYGVH

SEQ ID NO: 29 - protein, 9 aa, Artificial Sequence
  VH CDR2 definition under AbM method
VIWSDGSTT

SEQ ID NO: 30 - protein, 7 aa, Artificial Sequence
  VH CDR1 definition under Chothia method.
GFSLTSY

SEQ ID NO: 31 - protein, 5 aa, Artificial Sequence
  VH CDR2 definition under Chothia method.
WSDGS

SEQ ID NO: 32 - protein, 6 aa, Artificial Sequence
  VH CDR1 definition under Contact method.
TSYGVH

SEQ ID NO: 33 - protein, 12 aa, Artificial Sequence
  VH CDR2 definition under Contact method.
WLVVIWSDGSTT

SEQ ID NO: 34 - protein, 11 aa, Artificial Sequence
  VH CDR3 definition under Contact method.
ASHYGSTLAFA

SEQ ID NO: 35 - protein, 7 aa, Artificial Sequence
  VL CDR1 definition under Contact method
YSYLAWY

SEQ ID NO: 36 - protein, 10 aa, Artificial Sequence
  VL CDR2 definition under Contact method
LLVYNAKILA

SEQ ID NO: 37 - protein, 7 aa, Artificial Sequence
  VL CDR3 definition under Contact method
QHHYGTF

(19) United States
(12) Patent Application Publication
(10) Pub. No.: US 2006/0063209 A1
(43) Pub. Date: Mar. 23, 2006

(54) Engineered antibody fragment that irreversibly binds an antigen

(75) Inventors: Claude F. Meares, Davis, CA (US); Nathaniel G. Butlin, Davis, CA (US)

Correspondence Address: MORGAN, LEWIS & BOCKIUS LLP (SF), 2 PALO ALTO SQUARE, 3000 El Camino Real, Suite 700, PALO ALTO, CA, 94306, US

(73) Assignee: THE REGENTS OF THE UNIVERSITY OF CALIFORNIA, Oakland, CA (US)

(21) Appl. No.: 11/209,289

(22) Filed: Aug. 22, 2005

Related U.S. Application Data
(60) Provisional application No. 60/603,059, filed on Aug. 20, 2004.

(51) Int. Cl. G01N 33/567; C07K 16/46

(52) U.S. Cl. 435/7.2; 530/388.22; 530/391.1

(57) ABSTRACT

The present invention provides mutant antibodies with infinite affinity for a target antigen. The antibodies comprise a mutant amino acid at a position within or proximate to a complimentarity determining region of the antibody and a linker covalently bound to the mutant amino acid, the linker comprising a reactive functional group. Subsequent to binding an antigen, the reactive functional group is converted to a covalent bond by reaction with a group of complementary reactivity on the bound antigen. The invention also provides bispecific antibodies with infinite binding affinity that comprise a second domain that specifically binds a metal chelate. The invention further provides methods of using such antibodies to diagnose and treat diseases and conditions.

### CROSS-REFERENCES TO RELATED APPLICATIONS

This application is related to U.S. patent application Ser. No. 09/671,953, filed Sep. 27, 2000, and U.S. patent application Ser. No. 10/350,555, filed Jan. 23, 2003 and claims the benefit of U.S. Provisional Patent Application No. 60/603,059 filed Aug. 20, 2004, each of which is incorporated herein by reference in their entirety.

### STATEMENT AS TO RIGHTS TO INVENTIONS MADE UNDER FEDERALLY SPONSORED RESEARCH AND DEVELOPMENT

This invention was made with Government support under Grant Nos. CA 16861 and CA98207, awarded by the NIH/NCI to C. F. Meares. The Government has certain rights in this invention.

### BACKGROUND OF THE INVENTION

Over a million new cases of cancer will be diagnosed, and over half a million Americans will die from cancer this year. Although surgery can provide definitive treatment of cancer in its early stages, the eradication of metastases is crucial to the cure of more advanced disease. Chemotherapeutic drugs used in combinations provide the standard treatment for metastises and advanced disease. However, the side effects of these treatments seriously diminish the quality of life for cancer patients, and progressions and relapses following surgery and chemotherapy/radiation are common. Thus, despite the expenditure of large amounts of public and private resources over many years, better treatments for cancer are still sorely needed.

Post-genomic molecular approaches to cancer treatment promise to make cancer a treatable disease managed in much the same way as high blood pressure or diabetes. Hopefully, drugs will be available which are capable maintaining the cancer in check and/or even causing long term remission with a minimum of side effects.

Most pharmaceuticals available for cancer therapy are small molecules which traverse cell membranes and become widely distributed through the body. Unfortunately, the systemic use of such conventional antineoplastic drugs is associated with undesirable side effects arising from the lack of specificity, and hence the concommitant toxicity to normal cells. Naturally, the lack of specificity and toxic side effects limit the doses tolerated by a patient for treatment of the disease.

Thus, developing the technology to target therapeutic drugs to cancer cells while sparing normal cells, is the obvious goal for improved treatment of cancer. Macromolecules such as monoclonal antibodies and their derivatives or fragments, which bind to highly expressed tumor antigens and not significantly to normal cells, are the best candidates for targeted therapies.

To date, antibodies have in fact proved to be surprisingly effective therapeutics. There are now almost 60 antibodies approved for the treatment of diseases including cancer, and many more are in clinical trials. These antibody therapeutics are used in much the same way as injected small-molecule chemotherapeutics, except that due to the mass difference, grams of antibody are usually administered rather than milligrams of small molecule. Typical antibodies for cancer are Rituxan, which binds to the CD20 molecule on B cells, and Herceptin, which binds the Her2/neu epidermal growth factor receptor on breast cancer cells (Cragg M S, et al. Blood 2003 Feb. 1; 101(3):1045-52; and Albanell J, et al. Adv Exp Med Biol. 2003; 532:253-68).

Radioimmunotherapy provides further examples of the successful use of antibodies in cancer therapeutics. Radiolabeled antibodies have the advantage that they can be effective even in the face of defective host immune effector function (Press O W. Semin Oncol. 2003 April; 30(2 Suppl 4):10-21). Some potentially useful antibodies which have been conjugated to metal chelates for radioimmunotherapy include antibodies HMFG1 (Nicholson, S; et al. Oncology Reports 5, 223-226 (1998)), L6 (DeNardo, S J; et al., Journal of Nuclear Medicine 39, 842-849 (1998)), and Lym-1 (DeNardo, G L; et al., Clinical Cancer Research, 3: 71-79 (1997)). Two radiolabeled monoclonal antibodies that have been approved by the FDA for targeted radiotherapy of lymphoma (Campbell P, et al., Blood Rev. 2003; 17(3): 143-52; and Silverman D H, et al., Cancer Treat Rev. 2004; 30(2): 165-72) are ⁹⁰Y-labeled Zevalin, an IgG that targets CD20 (Li H, et al., J Biol. Chem. 2003; 278(43): 42427-34; and Witzig T E, et al., J Clin Oncol. 2002 May 15; 20(10):2453-63.), and ¹³¹I-labeled Bexxar, another IgG that targets CD20.

Unfortunately despite the successful use of pure radiation-delivery vehicles such as Zevalin and Bexxar, these antibody based drugs have serious shortcomings. Both are whole IgG molecules that remain in the circulation for days: they pass through the highly radiation-sensitive bone marrow throughout this period, and bone-marrow toxicity limits the dose of radiation that can be tolerated by patients.

In an attempt to overcome the bone marrow toxicity associated with radiolabeled drugs such as Zevalin and Bexxar, unlabled antibodies have been developed to pretarget a tumor and then capture a labeled small molecule. Thus, rather than carrying a radionuclide to a tumor, the antibody carries a receptor. For example, bispecific antibodies that can bind to tumors and to metal chelates have been developed (Stickney, Dwight R.; et al., Cancer Res. (1991), 51(24), 6650-5; Rouvier, Eric; et al., Horm. Res. (1997), 47(4-6), 163-167. and Cardillo T M, et al., Clin Cancer Res. 2004 May 15; 10(10):3552-61 and copending U.S. patent application Ser. No. 10/350,555 filed Jan. 23, 2003 which is herein incorporated by reference in its entirety). When pretargeted to tumors, these bispecific antibodies bind to antigens and remain on the target (for a while), providing receptors for metal chelates. Subsequent administration of small, hydrophilic metal chelates leads to their capture by the pretargeted chelate receptors. Uncaptured chelate molecules clear quickly through the kidneys and out of the body, leaving very little radioactivity in bone marrow or other normal tissues.

Unfortunately, the bound lifetimes of various indium chelates at 37° C. are in the 10-40 min range, and often a slow rate of dissociation is important for in vivo therapeutic targeting applications. Although the multivalent binding of antibody IgG molecules to cell surfaces can lead to bound lifetimes of several days and modern bifunctional chelating agents hold their metals for even longer periods, still, the most important challenge remaining is to safely increase the antibody-hapten bound lifetime.

One approach to solving the problem of short lived complexes and rapid dissociation rates has been to employ the long-lived (strept)avidin-biotin system in combination with biotinylated metal chelates (Chinol, M; et al., Nuclear Medicine Communications, 18, 176-182 (1997)). The biotin-avidin complex is unusually stable (K_{A}˜4×10⁹ M⁻¹) and does not dissociate into its components at a significant rate under normal circumstances. A biotin molecule will remain bound to streptavidin with a half-life of about 35 hr. The biotin-streptavidin association is adequately long-lived even for therapeutic applications, and highly promising preclinical results have been reported for cancer therapy. However, there is competition from natural biotin, and both hen egg avidin and bacterial streptavidin are immunogenic. Thus, in solving one problen, the biotin-avidin approach introduces another.

Thus, there remains a need in the art for target specific molecules that bind strongly to their target and which support a therapeutic moiety that remains intact for a sufficiently long time to permit accurate imaging and/or safe effective therapy.

As noted above antibodies are the first choice for specific cellular targeting of cancer therapeutics. Antibodies bind their targets with specificity and are readily manipulated protein scaffolds. Furthermore, antibodies can be produced to bind to an almost unlimited variety of natural and unnatural targets.

Unfortunately, a well-known problem in tumor targeting is that conventional, reversibly-binding antibodies with high affinity do not penetrate efficiently beyond the surface of a tumor (Fujimori, K., et al., Cancer Res. 49, 5656-5663; Yokota T, et al., Cancer Res. (1992) Jun. 15; 52(12):3402-8; Gregory P. Adams, Robert Schier Journal of Immunological Methods 231 1999 249-260; Adams G P, et al., Cancer Res. (2001) Jun. 15; 61(12):4750-5; and Graff C P, and Wittrup K D Cancer Res. 2003 Mar. 15; 63(6):1288-96). This is usually called the binding-site barrier, and its basis is the long bound lifetime exhibited by a high-affinity antibody on its target. Weakly binding antibodies, or more usually their monovalent fragments, do not share this problem because they bind and dissociate frequently (FIG. 33). However, for the same reason that they penetrate the tumor, weakly binding antibodies do not remain in the tumor long enough to deliver effective therapy.

The paradoxical need for ligands of simultaneous low and high affinity significantly limits the efficacy of antibodies for imaging and above all for therapy of solid tumors. Selective binding without dissociation solves an important practical problem in probe capture for tumor targeting, but may also present an obstacle to efficacious distribution of the ligand throughout the tumor. In contrast, weak binding permits tumor penetration without allowing the residence time necessary for effective therapy. Clearly, what is needed in the art is a composition that can combine the best of both worlds; strong specific binding and rapid association-dissociation sufficient to allow tumor penetration.

Fortunately, it has now been discovered that weak binding and infinite binding can be combined. The combination provides effective tumor penetration of antitumor antibodies or engineered antibody fragments, while at the same time providing binding affinity sufficient for imaging or therapy. Indeed, the present invention provides low-affinity anti-tumor antibodies that bind permanently to their targets, but only after many association-dissociation events. The antibody constructs infiltrate the tumor and eventually attach permanently to their targets. Thus, the invention provides antibodies that bind with infinite affinity for the treatment and diagnosis of disease.

### SUMMARY OF THE INVENTION

A common problem in targeting anti-tumor antibody therapeutics is that conventional, reversibly-binding antibodies that bind with high affinity do not penetrate efficiently beyond the surface of a tumor. This binding-site barrier problem has its basis in the long bound lifetime exhibited by a high-affinity antibody on its target. Although weakly binding antibodies, or their monovalent fragments, do not share this problem because they bind and dissociate frequently (FIG. 33), they also do not remain in the tumor long enough to deliver effective therapy. Thus, there is a need in the art for tumor targeting antibodies that can penetrate deep into a tumor and remain bound to the tumor long enough to deliver effective therapy.

The invention solves this, and other problems, by providing an engineered antibody fragment that is capable of forming a highly specific, covalent bond with its antigen in the natural biological environment. The irreversibly binding antibody fragment of the invention overcomes a fundamental limitation of monovalent antibody fragments used in cancer therapies, namely, the low affinity for the target antigen. Similarly, the invention overcomes the problem of insufficient tumor penetration as experienced by antibodies that strongly bind tumor antigens.

The invention provides a generalized methodology for engineering irreversibly binding antibody fragments that is particularly useful in that it can be applied to systems without prior knowledge of the protein structures or binding of the antibody fragment (scFv) and its antigen. Thus, in one embodiment the invention provides mutant antibodies comprising mutant polypeptide sequences. The mutant polypeptide sequences comprise a mutant amino acid at a position within or proximate to a complimentarity determining region of the antibody and a linker covalently bound to the mutant amino acid. The linker further comprises a reactive functional group that can form a covalent bond with a functional group of complementary reactivity on an antigen once the antigen once the antigen is bound by a mutant antibody.

In some embodiments the linker is a member selected from the group consisting of substituted or unsubstituted alkyl, substituted or unsubstituted heteroalkyl, substituted or unsubstituted aryl, substituted or unsubstituted heteroaryl, and substituted or unsubstituted heterocycloalkyl moieties.

In another embodiment, the mutant antibody comprises a first domain that specifically binds to an antigen e.g., a cell surface antigen. In a related embodiment, the antibody comprises a second domain that specifically binds a therapeutic or diagnostic agent e.g., metal chelate.

The invention also provides an antibody-antigen complex formed between a mutant antibody and an antigen to which the antibody specifically binds.

In one embodiment the invention provides an antibody-antigen complex wherein the mutant antibody is covalently bound to its antigen through the reactive group on the linker.

In another embodiment, the invention provides a method of forming an antibody antigen complex that does not dissociate under physiologically relevant conditions. The method comprises contacting the antigen with a mutant antibody comprising: (i) a mutant polypeptide sequence, including a mutant amino acid at a position within or proximate to a complementarity determining region of the antibody, wherein the mutant amino acid is not present in that position in the wild type antibody; and (ii) a linker covalently bound to the mutant amino acid. The linker includes a reactive functional group. The binding takes place under conditions appropriate to complex the antibody to the antigen. A covalent bond is formed between the reactive functional group and a group of complementary reactivity on the antigen, thereby forming an antigen-antibody complex.

The invention provides a significant improvement over conventional antibody and radioimmunotherapies. The smaller antibody fragments capitalize on their reduced mass with faster clearance and better solid tumor permeability, but unlike ordinary antibodies, the antibody fragments of the invention ultimately irreversibly bind their target. The irreversibly binding scFv antibody fragments of the invention provide prolonged residence time for therapeutic moieties while overcoming the disadvantages associated with whole antibody based therapies. An engineered antibody fragment of the invention is capable of specific covalent linkage to its antigen. Thus, it combines the best features of both whole antibody and antibody fragment therapies, providing fast clearance and high tumor permeability plus infinite antibody-antigen bound lifetime.

Other objects and advantages of the invention will be apparent to the skilled artisan from the detailed description that follows.

### BRIEF DESCRIPTION OF THE DRAWINGS

FIG. 1 Characteristics of an antibody with infinite affinity. (a) When the antibody and ligand are apart, their complementary reactive groups do not react significantly with other molecules in the medium. (b) When the ligand binds to the antibody, the effective concentrations of their complementary reactive groups are sharply elevated, and a permanent covalent link is formed. Michael addition of the mutant S95C sidechain to the acryl group of indium (S)-1-[p-(acrylamido)benzyl]ethylenediaminetetraacetate is shown. (c) The linked antibody-ligand complex cannot dissociate.

FIG. 2 Crystal structure of the CHA255 antibody-ligand complex, adapted from Love et al. Biochemistry 32, 10950-10959 (1993). Two residues in the wild-type antibody that are not directly involved in ligand binding but are favorably located close to the para-substituent of the ligand are light-chain residues S95 and N96 (Kabat positions 93 and 94). The ligand is modeled as indium (S)-1-[p-(acrylamido) benzyl]ethylenediaminetetraacetate, rather than the hydroxyethyl derivative used in the crystal structure determination.

FIG. 3 Ligands containing electrophilic substituents, tested with engineered Fab fragments S95C and N96C. AABE, (S)-1-[p-(acrylamido)benzyl]-EDTA; ABE, (S)-1-[p-(amino)benzyl]-EDTA; BABE, bromoacetamidobenzyl-EDTA; CABE, chloroacetamidobenzyl-EDTA; CpABE, (S)-1-p chloropropionamidobenzyl-EDTA. All were labeled with ¹¹¹In for the experiments.

FIG. 4 Whole-body clearance of indium-¹¹¹I labeled electrophilic derivatives of benzyl-EDTA from BALB/c mice after tail-vein injection. Here the objective was to find an electrophilic chelate that clears from the animal quickly when not captured by an engineered CHA255. As shown, ABE and AABE have apparent half-lives of about 4 h, while CABE has a half-life of 9 h, and BABE (the most reactive) 41 h. Apparently CABE and BABE indium chelates react significantly with biological nucleophiles and remain in the animals. AABE clears as completely—and almost as rapidly—as ABE (the non-reactive control in this experiment).

FIG. 5 Conjugation of electrophilic ¹¹¹In-benzyl-EDTA derivatives with engineered Fab S95C. Phosphorimage of 10-20% SDS-PAGE gel of samples of complete culture media incubated with ¹¹¹In-labeled (A) CABE, (B) CpABE, (C) AABE, and (D) ABE. The radiolabeled light chains migrate near 25 Kd. Structures of reagents are shown in FIG. 3.

FIG. 6 Demonstration that engineered Fab S95C retains the ligand-binding selectivity of the parent antibody. (a) Competition ELISA curves. Different metal-ABE chelates compete with an immobilized indium chelate for binding Fab S95C. (b) Competitive binding of a series of metal-ABE chelates, measured by blocking the covalent attachment of ¹¹¹In-AABE to Fab S95C. SDSPAGE gel bands show increased labeling of the light chain with decreasing competition.

FIG. 7 (a) Phosphorimage of a representative SDS-PAGE assay of the extent of permanent attachment of ¹¹¹In-AABE to the light chain of Fab S95C. (b)Kinetics of formation of the covalent bond between bound ¹¹¹In-AABE and Fab S95C, at 22° C., pH 7.4.

FIG. 8 DOTA (1,4,7,10-tetraazacyclododecane-N,N′,N″,N′″-tetraacetic acid) and two bifunctional analogs, (S)BAD ((S)-2-(4-(2-bromo)-acetamido)-benzyl)-DOTA) and (S)NBD ((S)-2-(4-nitrobenzyl)-DOTA).

FIG. 9 Relative binding of metal-(S)NBD complexes to antibody 2D12.5. A representative set of competitive binding curves obtained from ELISA experiments. Distance between midpoints is related to difference between dissociation constants. Error bars (representing the standard error of the mean) are shown but are generally smaller than the data points.

FIG. 10 Dependence of differences in the standard Gibbs Free Energy of binding on rare earth ionic radius shows thermodynamically elastic binding behavior between antibody 2D12.5 and rare earth-(S)NBD complexes. G values measured relative to Y-(S)NBD (open diamond symbol). Error bars represent standard error of the mean. The overall G° of binding for Y-(S)NBD is −45.7 kJ/mol.

FIG. 11 Crystal structure of the Y-(S)HETD-2D12.5 Fab complex.

FIG. 12 Comparison of the structures of (A) Antibody 2D12.5 bound to Y-(S)HETD and (B) Antibody 2D12.5 bound to Gd-(S)NBD. Each metal chelate is rendered as a stick model, with the metal as a sphere. The antibody is rendered as a surface, showing the binding cleft. At the bottom of the binding cleft in blue is Arg95(H) whose side chain forms a stabilizing salt bridge with a DOTA carboxylate; (clockwise from top right) are the side chain nitrogen atoms of Trp52(H), Trp96(L) and Asn100A(H), and the main chain amide nitrogen of Tyr98(H), which form hydrogen bonds to DOTA carboxylates. In each case the antibody structure is the same within experimental error, but the (S)HETD side chain is rotated 90° relative to the (S)NBD side chain.

FIG. 13 (A) Diagram showing the principal contacts between ligand and antibody in the 2D12.5-Y-(S)HETD complex, including five crystallographic water molecules. The Gd-(S)NBD is very similar. Figure designed with the aid of Ligplot (Wallace, A. C., et al. (1995) Protein Eng., 8, 127-134). (B) Three-dimensional structure showing the crystallographic bridging water molecules and the protein side chains within 5 Å of the Y-(S)HETD. Figure prepared with InsightII (Accelrys).

FIG. 14 Binding of yttrium chelates with different stereochemistry to antibody 2D12.5, showing that the antibody binds a chelate with the side chain in the R configuration approximately one order of magnitude less well than the S configuration. Yttrium-DOTA with no side chain, which is an equal mixture of R and S, is approximately in the middle. Error bars are smaller than the data symbols. Data were generated by competitive ELISA.

FIG. 15 The carboxylate oxygen atoms of Y-(S)HETD and Y-(R)HETD that do not coordinate to the metal are colored red and green, respectively, and are important for binding to antibody 2D12.5. Although these two molecules are enantiomers, the nature of the metal-complexed DOTA moiety allows for the two molecules and their non-coordinating carboxylate oxygen atoms to be almost superimposed.

FIG. 16 Site-directed cysteine mutations were designed using the crystal structure of 2D12.5 Fab bound to the Y-DOTA derivative, Y-HETD, which was modified in silico to the electrophilic acryl derivative (FIG. 17), Y-AABD (the p-substituent does not contact the protein). The native glycine residues 54, 55 and 56 in complementarity determining region 2 of the heavy chain appeared to be best suited for replacement with cysteine. The G54C mutant is shown.

FIG. 17 a) Scheme describing the permanent binding pair. b) Infinite binding is observed not only for ⁹⁰Y-AABD but also for ¹¹¹In-AABD, assayed by SDS-PAGE under denaturing conditions where only permanently bound complexes remain attached to the antibody. Increasing the concentration of unlabeled Y-NBD, a reversibly binding competitor, increasingly inhibits the infinite binding activity by preventing ⁹⁰Y-AABD or ¹¹¹In-AABD from accessing the binding site.

FIG. 18 The G54C mutant was preincubated in triplicate with 10 μM AABD complexes of Y³⁺, In³⁺ or Cu²⁺, a negative control N-(1-carbamoyl-2-(4-nitrophenyl)-ethyl)-acrylamide or N-(4-carboxymethylphenyl)-acrylamide, or just buffer, for 5 min, 20 min or 120 min at 37° C., pH 7.5. At the stated times ⁹⁰Y-AABD (1 μM) was added to each solution to compete for free G54C. Permanent binding was assayed by measuring band intensities after SDS-PAGE. a) Phosphorimage of gel. The fainter the bands, the greater the permanent binding by the unlabeled ligand. b) Crystal structures of Y-DOTA, In-DOTA-mono(p aminoanilide) (some atoms removed for clarity) and Cu-DOTA.

FIG. 19 a) Metal-AABD (10 μM) complexes were preincubated separately with aliquots of G54C for 5 min, followed by addition of 1 μM ⁹⁰Y-AABD, which competes for free G54C, and SDS-PAGE analysis. b) From quantitative phosphorimaging, the highest-affinity AABD complexes form permanent bonds with G54C with higher yields than more 3+weakly binding rare earth-AABD complexes whose ionic radii are slightly smaller (Lu Yb³⁺) or larger (Ce³⁺, La³⁺) than ideal.

FIG. 20 LC/MS analysis of Tb-AABD- and Tm-AABD-tagged G54C Fab peptide after enzymatic digestion with chymotrypsin. The labeled peptide was affinity purified with an immobilized 2D12.5 column prior to LC/MS analysis (Whetstone, P. A. et al. Bioconjugate Chem (2004), 15:3-6). Only the peptide containing the G54C engineered cysteine was labeled, and the ratio of Tb- and Tm-AABD labeled peptide was approximately equal as expected. MS2 analysis confirmed the sequence of the peptide and presence of either the Tb-AABD or Tm-AABD label.

FIG. 21 Kinetics of permanent bond formation between G54C Fab (1 μM) and 10 μM ⁹⁰Y-labeled YAABD. The addition of a high concentration of Y-NBD, a reversibly binding competitor, after 6 min competitively blocks the re-binding of any Y-AABD that dissociates without forming a permanent bond. The experiment containing no competitor, on the other hand, allows any Y-AABD molecules initially bound in an unproductive mode to rebind in an orientation favorable for permanent covalent bond formation until a maximum is reached.

FIG. 22 Assembled expression cassette of the Lym-1 single-chain antibody (sL1) used as template for all genetic mutants investigated for irreversible binding. The alpha-mating factor secretion signal (αMF) was used to target sL1 to the cellular secretion pathway of Pichia pastoris for ease of purification. The sL1 gene is a VH-(G4S)3-VL construct. The C-terminal epitope tag V5 was included for protein identification in heterologous expression media. A hexa-histidine tag was also engineered for downstream purification using immobilized metal affinity chromatography. Restriction sites included BglII and ApaI for the optional transfer of mutant gene constructs to S2 insect cell expression vectors.

FIG. 23 WAM¹¹¹ model of the Lym-1 single-chain antibody sL1.

FIG. 24 General outline of cross-linking strategy to produce an antibody that binds permanently to its protein target. Experiments with the target HLA-DR in vitro will be followed by experiments with Raji cells in culture.

FIG. 25 Gel shift assay of permanent attachment of sL1 to HLA-DR: western blot stained with anti-HLA-DR10β (antibody HL-40). The glycosylated β subunit of HLA-DR10 runs approximately 28-34 kDa, so the cross-linked sL1-HLA-DR10β should run at 57-63 kDa (bands in lanes 5 and 10). Lanes 2-6 are cysteine mutant F96C and lanes 7-11 are cysteine mutant T97C. Lanes 2 and 7 are sL1 mutant only; lanes 3 and 8 are sL1 mutant and HLA-DR10 target; lanes 4 and 9 are sL1 mutant and electrophile (no target); lanes 5 and 10 are the complete experiment: sL1 mutant, electrophile, and target; and lanes 6 and 11 are sL1 mutant with electrophile and recombinant human serum albumin as a negative control. Lnkr: nitrophenyl ester; Trgt: HLA-DR10.

FIG. 26 Control reaction of parental sL1 (no cysteine) compared to the T97C mutant. Lanes 2-6 are sL1 cysteine mutant T97C and lanes 7-11 are unmutated Lym-1 scFv. Permanent attachment to HLA-DR10β is seen with the conjugated cysteine mutant (lane 5) but not with the wild-type sL1 (lane 10). Experimental details are the same as for FIG. 25.

FIG. 27 Investigation of various linker reagents (carboxylic esters) with the sL1 mutant T97C. After 19 h incubation, the nitrophenyl ester shows strong reactivity (lane 6) followed by the phenyl ester (lane 8). All other reagents showed no reactivity above background. Western blotting with HLA-DR11β specific HL-40. Lane 2 is T97C only, lane 3 is HLA-DR prep only, lane 4 is T97C nitrophenylbromoacetate conjugate, lane 5 is identical to lane 4 except for the addition of excess human serum albumin as negative control.

FIG. 28 (from Adams et al., Cancer Res. 2001 Jun. 15; 61(12):4750-5, FIG. 3.) Immunohistochemical and immunofluorescence examination of the in vivo distribution of anti-HER-2/neu scFv molecules relative to the location of tumor vasculature in anephric SK-OV-3 tumor-bearing scid mice. a) low affinity (3.2×10⁻⁷ M) anti-HER-2/neu scFv displays a diffuse staining pattern relative to tumor blood vessels by immunohistochemistry. In b, immunofluorescence examination of nearby section of same tumor (3.2×10⁻⁷ M anti-HER-2/neu scFv and tumor blood vessels) again indicates diffusion from the blood vessels. In c, higher magnification of boxed area in B. In d, immunohistochemical examination of the localization of the high affinity (1.5×10⁻¹¹ M) anti-HER-2/neu scFv relative to tumor blood vessels reveals a focal staining pattern with short diffusion from the vascular bed. In E, immunofluorescence examination of nearby section of same tumor (1.5×10⁻¹¹ M anti-HER-2/neu scFv and tumor blood vessels again indicates a lack of diffusion from the tumor blood vessels. In f, higher magnification of boxed area in e. Original magnifications: ×10 (A, B, D, and E) and ×40 (C and F).

FIG. 29 Schemes for rate measurements. A). parental single-chain antibody (scFv) or unconjugated mutants; B). mutant scFv conjugated with non-reactive analog (amide); C). mutant scFv conjugated with cross-linker (ester). Ag: antigen.

FIG. 30 Genes for single-chain diabody T84.66-2D12.5 (A) and tandem scFv T84.66-2D12.5 (B). The N-terminal signal sequence (gray box), and the C-terminal V5 epitope and hexahistidyl tags are shown. Below: structures of folded single-chain diabody (A) and tandem scFv (B) protein molecules. The drawings suggest the rigidity of the single-1chain diabody and the flexible connection of the scFv fragments in the tandem scFv through the middle linker L5. Antigen-binding sites are highlighted as gray areas (Tina Kom, et al. J Gene Med (2004) δ: 642-651).

FIG. 31 DOTA derivatives that are to be synthesized for exploration of the permanent binding behavior of engineered 2D12.5 mutants.

FIG. 32 Schematic depiction contrasting the reversible binding of a wild-type (wt) Lym-1 single-chain (sL1) with the irreversible binding of an engineered sL1. The low affinity wt-sL1 reversibly binds its natural antigen HLA-DR10 with such low affinity to make it unfavorable as a targeting protein. An engineered sL1 that covalently binds HLA-DR10 via a reactive linker forms a complex with no effective dissociation even if the non-covalent antibody-antigen interactions are broken.

FIG. 33 General advantages of an irreversible scFv (i-scFv) over conventional IgG and scFv targeting strategies. IgG mediated targeting suffers from slow clearance of unbound IgG. When the IgG is labeled with a radionuclide, the slow clearance leads to significant irradiation of non-target tissues. IgG based strategies benefit however, from the bivalent nature of whole antibodies that naturally exhibit a long bound lifetime on the tumor cell surface. Engineered antibody fragments such as Fabs or scFvs have the advantage of fast clearance from circulation and much improved tumor penetration characteristics, however, they suffer from their monovalent nature with short bound lifetimes. An i-scFv would combine the best of both worlds with fast circulation clearance, good tumor penetration characteristics, and an infinite bound lifetime

FIG. 34 General structure of first round electrophiles used for crosslinking of sL1 with the target protein, HLA-DR1 Op. All compounds are commercially available and span a large reactivity with the most reactive R-group being nitrophenyl (a) down to a non-reactive acetate (g).

FIG. 35 DNA coding of expression cassettes constructed for Lym 1 single-chain (sL1) expression in Pichia pastoris. Two different secretion signals (aMF and PHO1) were initially investigated for expression efficiency and secreted yields of sL1. Both sequences also included C-terminal V5 and 6His epitopes. The DNA sequences are aligned above as aMFsL1XE (SEQ ID NO:1) and PHO1sL1XE (SEQ ID NO:2) respectively. A third construct (aMFsL1XN) (SEQ ID NO:3) was constructed that lacked the bulky C-terminal V5 epitope (Invitrogen).

FIG. 36 Translated expression cassette for Lym1 single-chain (sL1) expression in Pichia pastoris. Two different secretion signals (aMF and PHO1) were initially investigated for expression efficiency and secreted yields of sL1. Both sequences also included C-terminal V5 and 6His epitopes. The protein sequences are aligned above as aMFsL1XE (SEQ ID NO:4) and PHO1sL1XE (SEQ ID NO:5) respectively. A third construct (aMFsL1XN) (SEQ ID NO:6) was constructed that lacked the bulky C-terminal V5 epitope (Invitrogen).

FIG. 37 DNA (SEQ ID NO:7) and amino acid (SEQ ID NO:8) translation of the aMFsL1XE expression cassette. This construct was selected as the genetic template for subsequent mutations.

FIG. 38 Sequential and Kabat numbering alignment for expression cassette aMFsL1XE (SEQ ID NO:4). CDR residues based on Kabat definitions are highlighted in grey. Residue indicated with a dash (−) under Kabat sequence are expression cassette feature not associated with the sL1 coding region such as secretion signal, epitope tags and (G₄S)₃ linker.

FIG. 39. Selected features of expression cassette aMFsL1XE (SEQ ID NOs:4, 9-17).

FIG. 40. Representative DNA sequence alignment for V_{H}-CDR1 of sL1. Site-Directed mutagenesis was used to replace DNA sequence coding CDR residues with a cysteine residue. The five residues of the V_{H}-CDR1 (SYGVH) were each independently mutated to cysteine. See (SEQ ID NOs:7, 18-22). Although this alignment is specific for the V_{H}-CDR1, it is representative of all 6 CDRs present in the sL1 gene.

FIG. 41 Representative amino acid sequence alignment for V_{H}-CDR1 of sL1. Site-Directed mutagenesis was used to replace DNA sequence coding CDR residues with a cysteine residue. The five residues of the V_{H}-CDR1 (SYGVH) were each independently mutated to cysteine. See (SEQ ID NOs:4, 23-27). Although this alignment is specific for the V_{H}-CDR1, it is representative of all 6 CDRs present in the sL1 gene.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described below are those well known and commonly employed in the art. Standard techniques are used for nucleic acid and peptide synthesis. Generally, enzymatic reactions and purification steps are performed according to the manufacturer's specifications. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well known and commonly employed in the art. Standard techniques, or modifications thereof, are used for chemical syntheses and chemical analyses.

“Antibody” refers to a polypeptide encoded by an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition, i.e., an antigen recognition domain. As used herein, “antigen recognition domain” means that part of the antibody, recombinant molecule, the fusion protein, or the immunoconjugate of the invention which recognizes the target or portions thereof. Typically the antigen recognition domain comprises the variable region of the antibody or a portion thereof, e.g., one, two, three, four, five, six, or more hypervariable regions. The terms “V_{H}” or “VH” refer to the variable region of an immunoglobulin heavy chain, including an Fv, scFv, dsFv or Fab. The terms “V_{L}” or “VL” refer to the variable region of an immunoglobulin light chain, including an Fv, scFv, dsFv or Fab.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993). Thus, the term antibody, as used herein, also includes antibody fragments either produced by the modification of whole antibodies.

As used herein, “fragment” is defined as at least a portion of the variable region of the immunoglobulin molecule, which binds to its target, i.e. the antigen recognition domain or the antigen binding region. Some of the constant region of the immunoglobulin may be included. Examples of antibody functional fragments include, but are not limited to, complete antibody molecules, humanized antibodies, antibody fragments, such as Fv, single chain Fv (scFv), hypervariable regions ro complementarity determining regions (CDRs), V_{L} (light chain variable region), V_{H} (heavy chain variable region), Fab, F(ab)2′ and any combination of those or any other portion of an immunoglobulin peptide capable of binding to target antigen (see, e.g., Fundamental Immunology (Paul ed., 4th. 1999). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term antibody also includes bivalent or bispecific molecules, diabodies, triabodies, and tetrabodies. Bivalent and bispecific molecules are described in, e.g., Kostelny et al., J. Immunol. 148: 1547 (1992), Pack and Pluckthun, Biochemistry 31: 1579 (1992), Zhu et al. Protein Sci. 6: 781 (1997), Hu et al. Cancer Res. 56: 3055 (1996), Adams et al., Cancer Res. 53: 4026 (1993), and McCartney, et al., Protein Eng. 8: 301 (1995).

A “mutant antibody” refers to an antibody in which an amino acid at a selected position in the wild type antibody is absent or is replaced by a different amino acid. The mutant amino acid may be a single deletion or substitution, or may be a deletion or substitution of two or more amino acids. A “mutant amino acid” refers to an amino acid that is different from the amino acid present in the corresponding wild type peptide sequence.

A “humanized antibody” refers to an antibody in which the antigen binding loops, i.e., complementarity determining regions (CDRs), comprised by the V_{H} and V_{L} regions are grafted to a human framework sequence. Typically, the humanized antibodies have the same binding specificity as the non-humanized antibodies described herein. Techniques for humanizing antibodies are well known in the art and are described in e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al., Nature 321: 522 (1986); and Verhoyen et al., Science 239: 1534 (1988). Humanized antibodies are further described in, e.g., Winter and Milstein, Nature 349: 293 (1991).

The terms “complementarity determining region” or “CDR” or “hypervariable region”, refer to amino acid sequences within the variable regions of both the heavy and light chains of an antibody that function to recognize and bind specifically to antigen. Antibodies with different specificities have different complementarity determining regions, while antibodies of the exact same specificity have identical complementarity determining regions.

The term “antigen” refers to any substance that, as a result of coming into contact with appropriate cells, induces a state of sensitivity and/or immune responsiveness after a latent period (days or weeks) and that reacts in a demonstrable way with antibodies and/or immune cells of the sensitized subject in vitro or in vivo.

The phrase “infinite binding” or “binds infinitely” refers to a chemical interaction strong enough to endure beyond the time required for a diagnostic or therapeutic procedure to be performed. A therapeutic antibody binds an antigen with “infinite infinity” when it provides a greater therapeutic effect than an identical therapeutic antibody that binds the same antigen with “non-infinite binding affinity.” In a preferred embodiment, an antibody binds an antigen with “infinite affinity” when the binding of the antigen to the antibody results in the formation of a covalent bond between the antigen and the antibody. An antibody that binds with “infinite affinity”, may also be referred t an “infinite antibody”, and “infinite affinity antibody” or an “irreversible antibody”.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Rossolini et al. (1994) Mol. Cell. Probes 8:91-98). The term nucleic acid is used interchangeably with gene, cDNA, mRNA, oligonucleotide, and polynucleotide.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has at least 25% sequence identity. Alternatively, percent identity can be any integer from 25 to 100. More preferred embodiments include at least: 25%, 30%, 35%, 40%, 45%, 50%, 55%, 60%, 65%, 70%, 75%, 80%, 85%, 90%, 95%, or 99% or higher, compared to a reference sequence using the programs described herein, preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like. “Substantial identity” of amino acid sequences for these purposes normally means that a polypeptide comprises a sequence that has at least 40% sequence identity to the reference sequence. Preferred percent identity of polypeptides can be any integer from 40 to 100. More preferred embodiments include at least 60%, 65%, 70%, 75%, 80%, 85%, 90%, 95%, or 99%. Polypeptides which are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine.

Optimal alignment of sequences for comparison may be conducted by the local identity algorithm of Smith and Waterman (1981) Add. APL. Math. 2:482, by the identity alignment algorithm of Needleman and Wunsch (1970) J. Mol. Biol. 48:443, by the search for similarity method of Pearson and Lipman (1988) Proc. Natl. Acad. Sci. U.S.A. 85:2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

A preferred example of algorithms that are suitable for determining percent sequence identity and sequence similarity are the BLAST and BLAST 2.0 algorithms, which are described in Altschul et al. (1977) Nuc. Acids Res. 25:3389-3402 and Altschul et al. (1990) J. Mol. Biol. 215:403-410, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both

Another indication that nucleotide sequences are substantially identical is if two molecules hybridize to each other, or to a third nucleic acid, under moderately, and preferably highly, stringent conditions. Stringent conditions are sequence dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization.

Exemplary stringent hybridization conditions can be as following: 50% formamide, 5× SSC, and 1% SDS, incubating at 42° C., or, 5×SSC, 1% SDS, incubating at 65° C., with wash in 0.2× SSC, and 0.1% SDS at 65° C.

For the purpose of the invention, suitable “moderately stringent conditions” include, for example, prewashing in a solution of 5×SSC, 0.5% SDS, 1.0 mM EDTA (pH 8.0), hybridizing at 50° C.-65° C., 5×SSC overnight, followed by washing twice at 65° C. for 20 minutes with each of 2×, 0.5× and 0.2×SSC (containing 0.1% SDS). Such hybridizing DNA sequences are also within the scope of this invention. As used herein, “nucleic acid” means DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, and fluxionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications such as capping with a fluorophore or another moiety.

“Peptide,” “polypeptide” or “protein” refers to a polymer in which the monomers are amino acids and are joined together through amide bonds, alternatively referred to as a polypeptide. When the amino acids are α-amino acids, either the L-optical isomer or the D-optical isomer can be used. Additionally, unnatural amino acids, for example, β-alanine, phenylglycine and homoarginine are also included. Amino acids that are not gene-encoded may also be used in the present invention. Furthermore, amino acids that have been modified to include reactive groups may also be used in the invention. All of the amino acids used in the present invention may be either the D- or L-isomer. The L-isomers are generally preferred. In addition, other peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983). The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

“Reactive functional group,” as used herein refers to groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups alos include those used to prepare bioconjugates, e.g., N-hydroxysuccinimide esters, maleimides and the like. Methods to prepare each of these functional groups are well known in the art and their application to or modification for a particular purpose is within the ability of one of skill in the art (see, for example, Sandler and Karo, eds. ORGANIC FUNCTIONAL GROUP PREPARATIONS, Academic Press, San Diego, 1989).

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups, which are limited to hydrocarbon groups are termed “homoalkyl”.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′-represents both —C(O)₂R′— and —R′C(O)₂—.

The term “aryl” means, unless otherwise stated, a polyunsaturated, aromatic, hydrocarbon substituent, which can be a single ring or multiple rings (preferably from 1 to 3 rings), which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, —N—OR′, —NR′R″, —SR′, -halogen, —SiR′R″ R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″ R′″)═NR“ ”, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR′C(O)₂R′, —NR—C(NR′R″ R′″)═NR“ ”, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

The symbol [symbol] , whether utilized as a bond or displayed perpendicular to a bond indicates the point at which the displayed moiety is attached to the remainder of the molecule, solid support, etc.

“Non-covalent protein binding groups” are moieties that interact with an intact or denatured polypeptide in an associative manner. The interaction may be either reversible or irreversible in a biological milieu. The incorporation of a “non-covalent protein binding group” into a chelating agent or complex of the invention provides the agent or complex with the ability to interact with a polypeptide in a non-covalent manner. Exemplary non-covalent interactions include hydrophobic-hydrophobic and electrostatic interactions. Exemplary “non-covalent protein binding groups” include anionic groups, e.g., phosphate, thiophosphate, phosphonate, carboxylate, boronate, sulfate, sulfone, sulfonate, thiosulfate, and thiosulfonate.

The term “targeting moiety” is intended to mean a moiety that is (1) able to direct the entity to which it is attached (e.g., therapeutic agent or marker) to a target cell, for example to a specific type of tumor cell or (2) is preferentially activated at a target tissue, for example a tumor. The targeting group can be a small molecule, which is intended to include both non-peptides and peptides. The targeting group can also be a macromolecule, which includes saccharides, lectins, receptors, ligand for receptors, proteins such as BSA, antibodies, and so forth.

As used herein, an “immunoconjugate” means any molecule or ligand such as an antibody or growth factor (i.e., hormone) chemically or biologically linked to a cytotoxin, a radioactive agent, an anti-tumor drug or a therapeutic agent. The antibody or growth factor may be linked to the cytotoxin, radioactive agent, anti-tumor drug or therapeutic agent at any location along the molecule so long as the antibody is able to bind its target. Examples of immunoconjugates include immunotoxins and antibody conjugates.

As used herein, “selectively killing” means killing those cells to which the antibody binds.

As used herein, examples of “carcinomas” include bladder, breast, colon, larynx, liver, lung, ovarian, pancreatic, rectal, skin, spleen, stomach, testicular, thyroid, and vulval carcinomas.

As used herein, an “effective amount” is an amount of the antibody, immunoconjugate, which selectively kills cells or selectively inhibits the proliferation thereof.

As used herein, “therapeutic agent” means any agent useful for therapy including anti-tumor drugs, cytotoxins, cytotoxin agents, and radioactive agents.

As used herein, “anti-tumor drug” means any agent useful to combat cancer including, but not limited to, cytotoxins and agents such as antimetabolites, alkylating agents, anthracyclines, antibiotics, antimitotic agents, procarbazine, hydroxyurea, asparaginase, corticosteroids, mytotane (O,P′-(DDD)), interferons and radioactive agents.

As used herein, “a cytotoxin or cytotoxic agent” means any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

As used herein, “a radioactive agent” includes any radioisotope, which is effective in destroying a tumor. Examples include, but are not limited to, indium-111, Y-90, Lu-177, Sm-153, Er-169, Dy-165, Cu-67, cobalt-60 and X-rays. Additionally, naturally occurring radioactive elements such as uranium, radium, and thorium, which typically represent mixtures of radioisotopes, are suitable examples of a radioactive agent.

As used herein, “administering” means oral administration, intranasal administration, administration as a suppository, topical contact, intravenous, intraperitoneal, intramuscular, intralesional or subcutaneous administration, or the implantation of a slow-release device e.g., a miniosmotic pump, to the subject.

As used herein, “cell surface antigens” means any cell surface antigen which is generally associated with cells involved in a pathology (e.g., tumor cells), i.e., occurring to a greater extent as compared with normal cells. Such antigens may be tumor specific. Alternatively, such antigens may be found on the cell surface of both tumorigenic and non-tumorigenic cells. These antigens need not be tumor specific. However, they are generally more frequently associated with tumor cells than they are associated with normal cells.

As used herein, “tumor targeted antibody” means any antibody, which recognizes cell surface antigens on tumor (i.e., cancer) cells. Although such antibodies need not be tumor specific, they are tumor selective, i.e. bind tumor cells more so than it does normal cells.

As used herein, “pharmaceutically acceptable carrier” includes any material which when combined with the antibody retains the antibody's immunogenicity and non-reactive with the subject's immune systems. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods.

### DETAILED DESCRIPTION OF THE INVENTION AND THE PREFERRED EMBODIMENTS

A. The Compositions

The present invention provides compositions for delivering therapeutic and diagnostic agents directly to cells involved in a disease or condition. The compositions of the invention include antibodies with infinite binding affinity for their specific antigen. The antibodies comprise a mutant amino acid at a position within or proximate to a complimentarity determining region of the antibody, and further, a linker covalently bound to the mutant amino acid. The linker covalently bound to the mutant amino acid comprises a reactive functional group. Subsequent to binding of an antigen by the antibody, the reactive functional group is converted to a covalent bond by reaction with a group of complementary reactivity on the bound antigen. The invention also provides bispecific antibodies with infinite binding affinity that comprise a second domain that specifically binds a diagnostic or therapeutic agent e.g., a metal chelate. The invention further provides methods of using such antibodies to diagnose and treat diseases and conditions.

The present invention is illustrated by reference to the use of single chain Lym-1 antibodies as an exemplary embodiment. The use of single chain Lym-1 antibodies to illustrate the concept of the invention is not intended to define or limit the scope of the invention. Those of skill in the art will readily appreciate that the concepts underlying the compositions and methods described herein are equally applicable to any therapeutic or diagnostic agent to which an antibody can be raised (e.g., antitumor drugs, cytotoxins, etc.).

The specific recognition and binding of biological molecules by antibodies is fundamental to many phenomena in biology and medicine. Antibodies naturally associate with their ligands in a reversible manner, to form complexes held together by non-covalent molecular interactions.

For practical applications it is desirable to produce antibodies that bind very tightly to their ligands, because this extends the residence of antibodies on their targets. Approaches based on combinatorial molecular biology have been developed to increase the binding affinity (e.g., decrease the dissociation constant _{KD}=1/KA=k_{off}/kₒₙ, units M) of an engineered antibody fragment for its ligand (Hoogenboom, H. R. (1997) Trends in Biotechnology 15, 62-70; Rader, C. & Barbas, C. F. 3rd. (1997) Current Opinion in Biotechnology 8, 503-508; Crameri, A., Cwirla, S., & Stemmer, W. P. C. (1996) Nature Medicine 2, 100-102; Schier, R., et al., (1996) J. Mol. Biol. 255, 28-43; Chen, Y., et al., (1999) J. Mol. Biol. 293, 865-881; Yang, W.-P., et al., (1995) J. Mol. Biol. 254, 392-403; Boder, E. T., et al., (2000) Proc. Natl. Acad. Sci. USA 97, 10701-10705; Shreder, K. (2000) Methods 20, 372-379.). These methods generally succeed by reducing k_{off}, while having less effect upon kₒₙ, functionally increasing the affinity. While the half-life for ligand dissociation from a typical antibody with nanomolar affinity is likely to be on the order of an hour at most (Northrup, S. H.; and Erickson, H. P. Proc Natl Acad Sci USA 1992, 89, 3338-3342; Foote, J.; and Eisen, H. N. Proc Natl Acad Sci USA 1995, 92, 1254-1256; and Meyer, D. L.; et al., Bioconjugate Chem 1990, 1, 278-284) advances in combinatorial selection have produced much stronger affinities, into the picomolar or even femtomolar range, with half-lives for dissociation up to several days (Boder E T, et al., Proc Natl Acad Sci USA. 2000 Sep. 26;97(20):10701-5; Yang, W. P., et al. (1995) J Mol Biol 254, 392-403; Schier, R., et al., (1996) J Mol Biol 263, 551-567; and Pini, A., et al., (1998) J Biol Chem 273, 21769-21776).

Unfortunately however, as discussed above, very high affinity interactions between antibodies and tumor antigens impairs efficient tumor penetration by the antibodies. Therefore, the problem is not just to make antibodies with stronger binding affinity, but to make strong binders that are also able to effectively penetrate tumors.

In one embodiment, the invention therefore provides mutant antibodies comprising a mutant polypeptide sequence. The mutant polypeptide sequence comprises a mutant amino acid at a position within or proximate to a complimentarity determining region of the antibody and a linker covalently bound to the mutant amino acid. The linker comprises a reactive functional group that can form a covalent bond with a functional group of complementary reactivity on an antigen bound by a mutant antibody. Thus, the invention provides antibodies with infinite binding affinity.

The invention provides significant advantages over conventional antibodies within the scope of cancer therapeutics and imaging. Indeed, increasing the bound lifetime of a targeting antibody increases the effective dose of any therapeutic agent e.g., cytotoxic conjugate or diagnostic coupled with the infinite affinity antibody without increasing the patient dose to non-target tissues.

A promising method to prolong the lifetime of a complex indefinitely is to make a permanent covalent bond between its components. Indeed, permanent attachment of protein to ligand essentially prevents dissociation, extending the life of the complex, in a preferred embodiment, infinitely (k_{off}=0 therefore, K_{A} approaches infinity).

In an exemplary embodiment a reactive site is created on the antibody by engineering a cysteine at one of several potentially interesting locations (e.g., within a complementarity determining region (CDR) of the antibody). A small library of single-Cys mutants may thus be produced. The library can be tested against a small library of electrophilic reagents, differing in structure and reactivity, to determine the best pairs for use in applications.

The electrophilic chelates preferrably are able to pass through the circulation and bind to the targeted antibody. Thus, they should not react prematurely with nucleophiles normally present in blood. Nucleophiles of amino groups, for example the thiols on glutathione and other small molecules, and cysteine in albumin. The mild electrophiles on alkylating agents used in cancer chemotherapy (nitrogen mustards, ethyleneimine derivatives, mesylate esters, etc.) provide guidance concerning the practical limits of reactivity. Because of the high local concentrations of nucleophile and electrophile in the antibody ligand complex, significantly weaker electrophiles suffice for reaction to create a covalent bond than would be required under lower effective local concentrations.

As discussed by Fersht (Alan Fersht, ENZYME STRUCTURE AND MECHANISM, 2nd Ed., Freeman, New York, 1985, pp. 56-63), the effect of local concentration can be appreciated by comparing rate constants for the same chemical reaction between two separate reactants, and between two reactive groups joined by a linker: 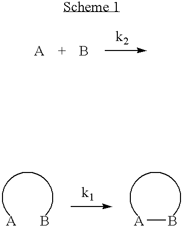
Effective local concentration of A in the presence of B in the unimolecular reaction=k₁/k₂ Fersht cites examples where the effective local concentration defined in this way is enormous (>10⁵ M). Thus, a hapten bearing a weakly reactive electrophile could diffuse intact through a dilute solution of nucleophiles, and still bind to its antibody and undergo attack by a nucleophilic sidechain.

Infinite affinity antibodies form a covalent attachment to any target to which they bind with measurable affinity, and that possess the required reaction partner. Indeed, ligands with even modest affinity for reversible binding to the parent antibody can be converted to infinite binders. However, the rate of covalent attachment may depend on the affinity. For example, a ligand with 10 nM affinity bound efficiently and permanently to mutant antibody 2D12.5 G54C and could not be significantly displaced by a competitor after 5 min; a ligand with 1 μM affinity bound permanently with approximately 50% yield after 5 min; and a ligand with 100 μM affinity bound permanently with approximately 70% yield after 2 hr. Without being bound by theory, it is likely that strong binders tend to have longer bound lifetimes in the complex and therefore are more likely to permanently attach at the first association, while weak binders associate and dissociate with the binding partners multiple times before forming a permanent link. Infinite affinity systems based on weak binders therefore have a combination of properties—both weak and covalent binding.

In one embodiment, the binding-site barrier is avoided by, for example, starting with a weak binder such as the Lym-1 single-chain antibody sL1 (which binds monovalently to its target HLADR with an affinity too weak to measure confidently, but probably in the micromolar range), and using the methods below to engineer a small set of permanent binders based on sL1, to produce constructs that will (1) bind and dissociate many times before (2) becoming permanently attached to HLA-DR on a cell surface. This is a combination of (1) the behavior characteristic of a weak binder, which should permeate a tumor, and (2) the behavior of an extremely strong binder, which should ultimately lock onto its targets throughout the tumor. This combination of good penetration and permanent binding is a unique and revolutionary aspect of this technology.

For purposes of illustration, the invention is described further by reference to an exemplary antibody-chelate pair. The description is for clarity of illustration, and is not intended to define or limit the scope of the present invention.

In an exemplary embodiment, a reactive site for attachment of a reactive linker is incorporated into an antibody by engineering a cysteine at one of several locations that are in or proximate to one of the CDR sequences of the antibody. The engineering is typically accomplished by site-directed mutagenesis of nucleic acids encoding the wild-type of the antibody. According to this method an array of mutant antibodies comprising a library of single-Cys mutants is prepared. Mutated antibodies, such as the single-Cys mutants can be prepared using methods that are now routine in the art (see, for example, Owens et al., Proceedings of the National Academy of Sciences USA 95: 6021-6026 (1998); Owens et al., Biochemistry 37: 7670-7675 (1998)). The library members are then tested against one or more electrophiles, differing in structure and reactivity, to determine the best pairs for a given purpose. As discussed above, the electrophiles preferably do not react prematurely with nucleophiles normally present in the blood.

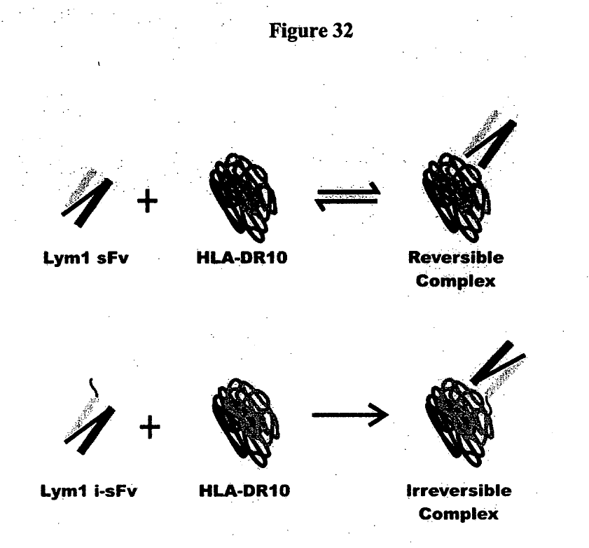

Thus, the invention provides an engineered antibody fragment capable of forming a highly specific, covalent bond with its antigen in the natural biological environment. As outlined in FIG. 32, an irreversible single-chain antibody (i-scFv) overcomes a fundamental limitation of monovalent antibody fragments, which is the low affinity for the target antigen.

The invention applies broadly to many antibody-antigen pairs, particularly when the antigen is a protein. In an exemplary embodiment the invention provides an irreversible Lym-1 single-chain antibody fragment (scFv or sFv). However, the methodology for engineering an irreversible antibody fragment can readily be applied to systems without prior specific knowledge of the protein structures or binding orientation of the scFv and its antigen.

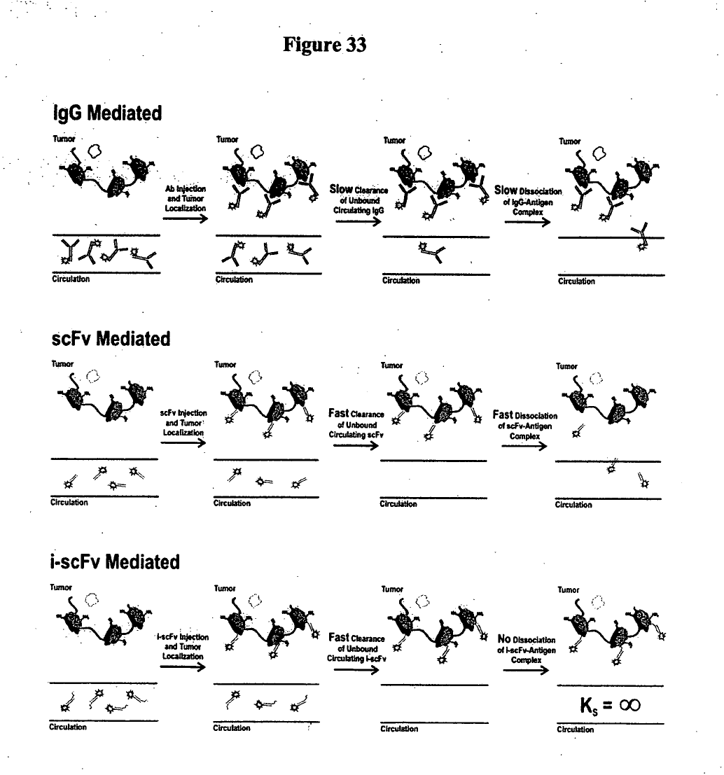

A significant improvement over conventional radioimmunotherapy (RAIT) approaches is expected with an irreversibly binding scFv targeting antibody. Whole antibody based targeting strategies possess sufficient tumor residence lifetimes, but suffer from slow circulation clearance and poor tumor penetration. Smaller antibody fragments capitalize on their reduced mass with faster clearance and better solid tumor permeability, but have reduced bound lifetimes severely hindering their therapeutic effectiveness. An engineered antibody fragment capable of specific covalent linkage to its antigen aims to combine the best features of both, with fast clearance and high tumor permeability plus infinite bound lifetime (see FIG. 33).

1. The Antibodies

The present invention provides antibodies that specifically bind to antigens. For preparation of monoclonal or polyclonal antibodies, any technique known in the art can be used (see, e.g., Kohler & Milstein, Nature 256: 495-497 (1975); Kozbor et al., Immunology Today 4: 72 (1983); Cole et al., pp. 77-96 in MONOCLONAL ANTIBODIES AND CANCER THERAPY, Alan R. Liss, Inc. (1985)).

Methods of producing of polyclonal antibodies are known to those of skill in the art. In an exemplary method, an inbred strain of mice (e.g., BALB/C mice) or rabbits is immunized with the antigen using a standard adjuvant, such as Freund's adjuvant, and a standard immunization protocol. Alternatively, or in addition to the use of an adjuvant, the antigen is coupled to a carrier that is itself immunogenic (e.g., keyhole limpet hemocyanin (“KLH”). The animal's immune response to the immunogen preparation is monitored by taking test bleeds and determining the titer of reactivity to the immunogen. When appropriately high titers of antibody to the immunogen are obtained, blood is collected from the animal and antisera are prepared. Further fractionation of the antisera to enrich for antibodies reactive to the protein can be done if desired.

Monoclonal antibodies are obtained by various techniques familiar to those skilled in the art. Briefly, spleen cells from an animal immunized with a desired antigen are immortalized, commonly by fusion with a myeloma cell (see, for example, Kohler & Milstein, Eur. J. Immunol. 6: 511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse et al., Science 246: 1275-1281 (1989).

Monoclonal antibodies and polyclonal sera are collected and titered against the immunogen in an immunoassay, for example, a solid phase immunoassay with the immunogen immobilized on a solid support. Typically, polyclonal antisera with a titer of 10⁴ or greater are selected and tested for cross reactivity against different antigens, using a competitive binding immunoassay. Specific polyclonal antisera and monoclonal antibodies will usually bind with a K_{d} of at least about 0.1 mM, more usually at least about 1 μM, preferably, at least about 0.1 μM or better, and most preferably, 0.01 μM or better.

Techniques for the production of single chain antibodies (U.S. Pat. No. 4,946,778) can be adapted to produce antibodies to antigens and other diagnostic, analytical and therapeutic agents. Also, transgenic mice, or other organisms such as other mammals, may be used to express humanized antibodies. Alternatively, phage display technology can be used to produce and identify antibodies and heteromeric Fab fragments that specifically bind to selected antigens (see, e.g., McCafferty et al., Nature 348: 552-554 (1990); Marks et al., Biotechnology 10: 779-783 (1992)).

In an exemplary embodiment, an animal, such as a rabbit or mouse is immunized with an antigen, or an immunogenic construct. The antibodies produced as a result of the immunization are preferably isolated using standard methods.

In a still further preferred embodiment, the antibody is a humanized antibody. “Humanized” refers to a non-human polypeptide sequence that has been modified to minimize immunoreactivity in humans, typically by altering the amino acid sequence to mimic existing human sequences, without substantially altering the function of the polypeptide sequence (see, e.g., Jones et al., Nature 321: 522-525 (1986), and published UK patent application No. 8707252).

In another preferred embodiment, the present invention provides an antibody, as described above, further comprising a member selected from detectable labels, biologically active agents and combinations thereof attached to the antibody.

When the antibody is conjugated to a detectable label, the label is preferably a member selected from the group consisting of radioactive isotopes, fluorescent agents, fluorescent agent precursors, chromophores, enzymes and combinations thereof. Methods for conjugating various groups to antibodies are well known in the art. For example, a detectable label that is frequently conjugated to an antibody is an enzyme, such as horseradish peroxidase, alkaline phosphatase, β-galactosidase, and glucose oxidase.

In an exemplary embodiment of the present invention, horseradish peroxidase is conjugated to an antibody raised against an antigen. In this embodiment, the saccharide portion of the horseradish peroxidase is oxidized by periodate and subsequently coupled to the desired immunoglobin via reductive amination of the oxidized saccharide hydroxyl groups with available amine groups on the immunoglobin.

Methods of producing antibodies labeled with small molecules, for example, fluorescent agents, are well known in the art. Fluorescent labeled antibodies can be used in immunohistochemical staining (Osborn et al., Methods Cell Biol. 24: 97-132 (1990); in flow cytometry or cell sorting techniques (Ormerod, M. G. (ed.), FLOW CYTOMETRY. A PRACTICAL APPROACH, IRL Press, New York, 1990); for tracking and localization of antigens, and in various double-staining methods (Kawamura, A., Jr., FLUORESCENT ANTIBODY TECHNIQUES AND THEIR APPLICATION, Univ. Tokyo Press, Baltimore, 1977).

Many reactive fluorescent labels are available commercially (e.g., Molecular Probes, Eugene, Oreg.) or they can be synthesized using art-recognized techniques. In an exemplary embodiment, an antibody of the invention is labeled with an amine-reactive fluorescent agent, such as fluorescein isothiocyanate under mildly basic conditions. For other examples of antibody labeling techniques, see, Goding, J. Immunol. Methods 13: 215-226 (1976); and in, MONOCLONAL ANTIBODIES: PRINCIPLES AND PRACTICE, pp. 6-58, Academic Press, Orlando (1988).

In some embodiments, the antibodies are mutant antibodies that have infinite affinity for a target antigen. The antibodies comprise a mutant amino acid at a position within or proximate to a complimentarity determining region of the antibody and a linker covalently bound to the mutant amino acid. Prior to constructing the mutagenized antibodies of the invention, it is often useful to prepare the wild-type antibody from an isolated nucleic acid encoding an antibody or a portion of an antibody of the invention. In a further preferred embodiment, the antibody fragment is an Fᵥ fragment. Fᵥ fragments of antibodies are heterodimers of antibody V_{H} (variable region of the heavy chain) and V_{L} domains (variable region of the light chain). They are the smallest antibody fragments that contain all structural information necessary for specific antigen binding. Fᵥ fragments are useful for diagnostic and therapeutic applications such as imaging of tumors or targeted cancer therapy. In particular, because of their small size, Fᵥ fragments are useful in applications that require good tissue or tumor penetration, because small molecules penetrate tissues much faster than large molecules (Yokota et al., Cancer Res., 52: 3402-3408 (1992)).

The heterodimers of heavy and light chain domains that occur in whole IgG, for example, are connected by a disulfide bond, but Fᵥ fragments lack this connection. Although native unstabilized Fᵥ heterodimers have been produced from unusual antibodies (Skerra et al., Science, 240: 1038-1041 (1988); Webber et al., Mol. Immunol. 4: 249-258 (1995), generally Fᵥ fragments by themselves are unstable because the V_{H} and V_{L} domains of the heterodimer can dissociate (Glockshuber et al., Biochemistry 29: 1362-1367 (1990)). This potential dissociation results in drastically reduced binding affinity and is often accompanied by aggregation.

Solutions to the stabilization problem have resulted from a combination of genetic engineering and recombinant protein expression techniques. Such techniques are of use in constructing the antibodies of the present invention. The most common method of stabilizing Fᵥs is the covalent connection of V_{H} and V_{L} by a flexible peptide linker, which results in single chain Fᵥ molecules (scFv; see, Bird et al., Science 242: 423-426 (1988); Huston et al., Proc. Natl. Acad. Sci. USA 16: 5879-5883 (1988)). The scFᵥs are generally more stable than Fᵥs alone.

Another way to generate stable recombinant Fᵥs is to connect V_{H} and V_{L} by an interdomain disulfide bond instead of a linker peptide; this technique results in disulfide stabilized Fᵥ (dsFᵥ). The dsFᵥs solve many problems that can be associated with scFᵥs: they are very stable, often show full antigen binding activity, and sometimes have better affinity than scFᵥs (Reiter et al., Int. Cancer 58: 142-149 (1994)). Thus, in another preferred embodiment, the antibody of the invention is a dsFᵥs

Peptide linkers, such as those used in the expression of recombinant single chain antibodies, may be employed as the linkers and connectors of the invention. Peptide linkers and their use are well known in the art. (See, e.g., Huston et al., 1988; Bird et al., 1983; U.S. Pat. No. 4,946,778; U.S. Pat. No. 5,132,405; and Stemmer et al., Biotechniques 14:256-265 (1993)). The linkers and connectors are flexible and their sequence can vary. Preferably, the linkers and connectors are long enough to span the distance between the amino acids to be joined without putting strain on the structure. For example, the linker (gly₄ser)₃ is a useful linker because it is flexible and without a preferred structure (Freund et al., Biochemistry 33: 3296-3303 (1994)).

After the stabilized immunoglobin has been designed, a gene encoding at least Fᵥ or a fragment thereof is constructed. Methods for isolating and preparing recombinant nucleic acids are known to those skilled in the art (see, Sambrook et al., Molecular Cloning. A Laboratory Manual (2d ed. 1989); Ausubel et al., Current Protocols in Molecular Biology (1995)).

The present invention provides for the expression of nucleic acids corresponding to essentially any antibody that can be raised against an antigen, and the modification of that antibody to include a reactive site.

Those of skill in the art will understand that substituting selected codons from the sequences of the invention with equivalent codons is within the scope of the invention. Oligonucleotides that are not commercially available are preferably chemically synthesized according to the solid phase phosphoramidite triester method first described by Beaucage & Caruthers, Tetrahedron Letts. 22: 1859-1862 (1981), using an automated synthesizer, as described in Van Devanter et. al., Nucleic Acids Res. 12: 6159-6168 (1984). Purification of oligonucleotides is preferably by either native acrylamide gel electrophoresis or by anion-exchange HPLC as described in Pearson & Reanier, J. Chrom. 255: 137-149 (1983). The sequence of the cloned genes and synthetic oligonucleotides can be verified after cloning using art-recognized methods, e.g., the chain termination method for sequencing double-stranded templates of Wallace et al., Gene 16: 21-26 (1981).

One preferred method for obtaining specific nucleic acid sequences combines the use of synthetic oligonucleotide primers with polymerase extension or ligation on a mRNA or DNA template. Such a method, e.g., RT, PCR, or LCR, amplifies the desired nucleotide sequence, which is often known (see, U.S. Pat. Nos. 4,683,195 and 4,683,202). Restriction endonuclease sites can be incorporated into the primers. Amplified polynucleotides are purified and ligated into an appropriate vector. Alterations in the natural gene sequence can be introduced by techniques such as in vitro mutagenesis and PCR using primers that have been designed to incorporate appropriate mutations.

A particularly preferred method of constructing the immunoglobulin is by overlap extension PCR. In this technique, individual fragments are first generated by PCR using primers that are complementary to the immunoglobulin sequences of choice. These sequences are then joined in a specific order using a second set of primers that are complementary to “overlap” sequences in the first set of primers, thus linking the fragments in a specified order. Other suitable Fᵥ fragments can be identified by those skilled in the art. The immunoglobulin, e.g., Fᵥ, is inserted into an “expression vector,” “cloning vector,” or “vector.” Expression vectors can replicate autonomously, or they can replicate by being inserted into the genome of the host cell. Often, it is desirable for a vector to be usable in more than one host cell, e.g., in E. coli for cloning and construction, and in an insect cell or a mammalian cell for expression. Additional elements of the vector can include, for example, selectable markers, e.g., tetracycline resistance or hygromycin resistance, which permit detection and/or selection of those cells transformed with the desired polynucleotide sequences (see, e.g., U.S. Pat. No. 4,704,362). The particular vector used to transport the genetic information into the cell is also not particularly critical. Any suitable vector used for expression of recombinant proteins host cells can be used. In a preferred embodiment, a Pichia pastoris system is used for the expression of an scFv comprising sequences of the complementarity determining regions from the V_{H} and V_{L} chains of an antibody.

Expression vectors typically have an expression cassette that contains all the elements required for the expression of the polynucleotide of choice in a host cell. A typical expression cassette contains a promoter operably linked to the polynucleotide sequence of choice. The promoter used to direct expression of the nucleic acid depends on the particular application, for example, the promoter may be a prokaryotic or eukaryotic promoter depending on the host cell of choice. The promoter is preferably positioned about the same distance from the heterologous transcription start site as it is from the transcription start site in its natural setting. As is known in the art, however, some variation in this distance can be accommodated without loss of promoter function.

Promoters include any promoter suitable for driving the expression of a heterologous gene in a host cell, including those typically used in standard expression cassettes. In addition to the promoter, the recombinant protein gene will be operably linked to appropriate expression control sequences for each host. For E. coli this includes a promoter such as the T7, trp, tac, lac or lambda promoters, a ribosome binding site, and preferably a transcription termination signal. For eukaryotic cells, the control sequences will include a promoter and preferably an enhancer derived from immunoglobulin genes, SV40, cytomegalovirus, etc., and a polyadenylation sequence, and may include splice donor and acceptor sequences.

The vectors can be transferred into the chosen host cell by well-known methods such as calcium chloride transformation for E. coli and calcium phosphate treatment or electroporation for insect cells or mammalian cells. Cells transformed by the plasmids can be selected by resistance to antibiotics conferred by genes contained on the plasmids, such as the amp, gpt, neo and hyg genes. One of skill in the art will appreciate that vectors comprising DNA encoding the V_{L} chain of an antibody and vectors comprising DNA encoding the V_{H} chain of an antibody can conveniently be separately transfected into different host cells. Alternately vectors comprising DNA encoding the V_{L} chain of an antibody and vectors comprising DNA encoding the V_{H} chain of an antibody may be cotransfected into the same host cells.

The antibodies and antibody fragments of the invention can be expressed in a variety of host cells, including E. coli, other bacterial hosts, yeast, insect cells such as S2 cells, and various higher eukaryotic cells such as the COS, CHO, and HeLa cells lines and myeloma cell lines. Methods for refolding single chain polypeptides expressed in bacteria such as E. coli have been described, are well-known and are applicable to the wild-type anti-chelate polypeptides. (See, e.g., Buchner et al., Analytical Biochemistry 205: 263-270 (1992); Pluckthun, Biotechnology 9: 545 (1991); Huse et al., Science 246: 1275 (1989) and Ward et al., Nature 341: 544 (1989)).

Often, functional protein from E. coli or other bacteria is generated from inclusion bodies and requires the solubilization of the protein using strong denaturants, and subsequent refolding. In the solubilization step, a reducing agent must be present to dissolve disulfide bonds as is well-known in the art. Renaturation to an appropriate folded form is typically accomplished by dilution (e.g. 100-fold) of the denatured and reduced protein into refolding buffer.

Once expressed, the recombinant proteins can be purified according to standard procedures of the art, including ammonium sulfate precipitation, affinity columns, column chromatography, and the like (see, generally, Scopes, PROTEIN PURIFICATION (1982)). The recombinant proteins can be separated from other proteins on the basis of their size, net surface charge, hydrophobicity, and affinity for ligands. In a preferred embodiments, the recombinant proteins comprise tags that facilitate column purification (e.g., tags comprising at least 2, 3, 4, 6, 8, or 8 histidine residues). Suitable columns include, for example, charge induction chromatography columns (HClCC), thiolphilic columns, ion exchange columns, gel filtration columns, immobilized metal affinity columns (IMAC), immunoaffinity columns, and combinations thereof. In some embodiments, DOTA complexes, ABD complexes, BAD complexes, NBD complexes, and AABD complexes, and combinations thereof can conveniently be used as affinity components of the columns (see, e.g., Example 6 below). It will be apparent to one of skill that chromatographic techniques can be performed at any scale and using equipment from many different manufacturers. It will also be apparent to one of skill in the art that additional processing of the recombinant proteins may be performed. For example, a reactive site on the protein or polypeptide may be treated to deblock the thiol groups using methods known in the art and described in, e.g., Stimmel et al., J. Biol. Chem. 275:30445-30450 (2000). Substantially pure compositions of at least about 90 to 95% homogeneity are preferred, and those of 98 to 99% or more homogeneity are most preferred for pharmaceutical uses. Once purified, partially or to homogeneity as desired, the polypeptides may then be used therapeutically and diagnostically.

a. Bispecific Antibodies

In another preferred embodiment, the present invention provides for a reactive antibody that is bispecific for both a metal chelate and a targeting reagent or a target tissue, such as a tumor. Bispecific antibodies (BsAbs) are antibodies that have binding specificities for at least two different antigens. Bispecific antibodies can be derived from full length antibodies or antibody fragments (e.g. SscFv)₂ bispecific antibodies). In a preferred embodiment, the bispecific antibody recognizes a DOTA complex (e.g., Y-, La-, Ce-, Pr-, Nd-, Sm-, Eu-, Gd-, Tb-, Dy-, Ho-, Er-, Ym-, Yb-, Lu-, Pm-, Ac-, Pa-, Am-, Sc-, Sr-, In-, Ti-, Bi-DOTA), an AABD complex (e.g., Y-, La-, Ce-, Pr-, Nd-, Sm-, Eu-, Gd-, Tb-, Dy-, Ho-, Er-, Ym-, Yb-, Lu-, Pm-, Ac-, Pa-, Am-, Sc-, Sr-, In-, Ti-, Bi-AABD), a BAD complex (e.g., Y-, La-, Ce-, Pr-, Nd-, Sm-, Eu-, Gd-, Tb-, Dy-, Ho-, Er-, Ym-, Yb-, Lu-, Pm-, Ac-, Pa-, Am-, Sc-, Sr-, In-, Ti-, Bi-BAD), an ABD complex (e.g., Y-, La-, Ce-, Pr-, Nd-, Sm-, Eu-, Gd-, Tb-, Dy-, Ho-, Er-, Ym-, Yb-, Lu-, Pm-, Ac-, Pa-, Am-, Sc-, Sr-, In-, Ti-, Bi-ABD), or a NBD complex (e.g., Y-, La-, Ce-, Pr-, Nd-, Sm-, Eu-, Gd-, Tb-, Dy-, Ho-, Er-, Ym-, Yb-, Lu-, Pm-, Ac-, Pa-, Am-, Sc-, Sr-, In-, Ti-, Bi-NBD), including reactive DOTA, AABD, BAD, ABD, and NBD complexes, of the invention and a human cancer cell (e.g., a carcinoma cell, a sarcoma cell, a colon cancer cell, stomach cancer cell, a liver cancer cell, a pancreatic cancer cell, a lung cancer cell, an ovarian cancer cell, a prostate cancer cell, a breast cancer cell, a skin cancer cell (e.g., a melanoma cell).

Methods for making bispecific antibodies are known in the art. Traditional production of full-length bispecific antibodies is based on the co-expression of two immunoglobulin heavy chain-light chain pairs, where the two chains have different specificities (Millstein and Cuello, Nature 305: 537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, published May 13, 1993, and in Traunecker et al., EMBO J. 10: 3655-3659 (1991)).

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. One of skill in the art will appreciate that any immunoglobulin heavy chain known in the art may be fused to an antibody variable domain with the desired binding specificity. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690 published Mar. 3, 1994. For further details of generating bispecific antibodies (see, for example, Suresh et al., Methods in Enzymology 121: 210 (1986)).

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al. (Science 229: 81 (1985)) describe a procedure wherein intact antibodies are proteolytically cleaved to generate F(ab′)₂ fragments. The fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the BsAb. The BsAbs produced can be used as agents for the selective immobilization of enzymes.

Recent progress has facilitated the direct recovery of Fab′—SH fragments from E. coli, which can be chemically coupled to form bispecific antibodies. Shalaby et al., J. Ex. Med., B 217-225 (1992) describe the production of a fully humanized BsAb F(ab′)₂ molecule. Each Fab′ fragment was separately secreted from E. coli and subjected to directed chemical coupling in vitro to form the BsAb. The BsAb thus formed was able to bind to cells overexpressing the HER2 receptor and normal human T cells, as well as trigger the lytic activity of human cytotoxic lymphocytes against human breast tumor targets. See also, Rodrigues et al., Int. J. Cancers, (Suppl.) 7: 45-50 (1992).

Various techniques for making and isolating BsAb fragments directly from recombinant cell culture have also been described and are useful in practicing the present invention. For example, bispecific F(ab′)₂ heterodimers have been produced using leucine zippers. Kostelny et al., J. Immunol., 148(5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. The “diabody” technology described by Hollinger et al., Proc. Natl. Acad. Sci. (USA), 90: 6444-6448 (1993) has provided an alternative mechanism for making BsAb fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making BsAb fragments by the use of single-chain Fv (sFv) dimers has also been reported (see, Gruber et al., J. Immunol., 152: 5368 (1994)). Gruber et al., designed an antibody which comprised the V_{H} and V_{L} domains of a first antibody joined by a 25-amino-acid-residue linker to the V_{H} and V_{L} domains of a second antibody. The refolded molecule bound to fluorescein and the T-cell receptor and redirected the lysis of human tumor cells that had fluorescein covalently linked to their surface.

The present invention also provides bispecific antibodies that include a mutant antibody that binds to metal chelates. The mutant antibodies are prepared by any method known in the art, most preferably by site directed mutagenesis of a nucleic acid encoding the wild-type antibody (see e.g., copending commonly owned U.S. patent application Ser. No. 09/671,953 which is herein incorporated by reference in its entirety).

b. Site-Directed Mutagenesis

The elements of the discussion above are also broadly applicable to aspects and embodiments of the invention in which site directed mutagenesis is used to produce mutant antibodies. The concept of site-directed mutagenesis as it applies to the present invention is discussed in greater detail to supplement, not to replace the discussion above.

The mutant antibodies are suitably prepared by introducing appropriate nucleotide changes into the DNA encoding the polypeptide of interest, or by in vitro synthesis of the desired mutant antibody. Such mutants include, for example, deletions from, or insertions or substitutions of, residues within the amino acid sequence of the polypeptide of interest so that it contains the proper epitope and is able to form a covalent bond with a reactive metal chelate. Any combination of deletion, insertion, and substitution is made to arrive at the final construct, provided that the final construct possesses the desired characteristics. The amino acid changes also may alter post-translational processes of the polypeptide of interest, such as changing the number or position of glycosylation sites. Moreover, like most mammalian genes, the antibody can be encoded by multi-exon genes.

In one embodiment, the variants are amino acid substitution variants. These variants have at least one amino acid residue in the polypeptide molecule removed and a different residue inserted in its place.

The nucleic acid molecules encoding amino acid sequence mutations of the antibodies of interest are prepared by a variety of methods known in the art. These methods include, but are not limited to, preparation by oligonucleotide-mediated (or site-directed) mutagenesis, PCR mutagenesis, and cassette mutagenesis of an earlier prepared variant or a non-variant version of the polypeptide on which the variant herein is based.

Oligonucleotide-mediated mutagenesis is a preferred method for preparing substitution, deletion, and insertion antibody mutants herein. This technique is well known in the art as described by Ito et al., Gene 102:67-70 (1991) and Adelman et al., DNA 2: 183 (1983). Briefly, the DNA is altered by hybridizing an oligonucleotide encoding the desired mutation to a DNA template, where the template is the single-stranded form of a plasmid or bacteriophage containing the unaltered or native DNA sequence of the polypeptide to be varied. After hybridization, a DNA polymerase is used to synthesize an entire second complementary strand of the template that will thus incorporate the oligonucleotide primer, and will code for the selected alteration in the DNA.

Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al., Proc. Natl. Acad. Sci. USA, 75: 5765 (1978).

The DNA template can be generated by those vectors that are either derived from bacteriophage M13 vectors (e.g., the commercially available M13 mp18 and M13 mp19 vectors are suitable), or those vectors that contain a single-stranded phage origin of replication as described by Viera et al. Meth. Enzymol., 153: 3 (1987). Thus, the DNA that is to be mutated may be inserted into one of these vectors to generate single-stranded template. Production of the single-stranded template is described in Sections 4.21-4.41 of Sambrook et al., supra. Alternatively, single-stranded DNA template is generated by denaturing double-stranded plasmid (or other) DNA using standard techniques.

Mutations in the V_{H} and V_{L} domains may be introduced using a number of methods known in the art. These include site-directed mutagenesis strategies.

The PCR products are subcloned into suitable cloning vectors that are well known to those of skill in the art and commercially available. Clones containing the correct size DNA insert are identified, for example, agarose gel electrophoresis. The nucleotide sequence of the heavy or light chain coding regions is then determined from double stranded plasmid DNA using the sequencing primers adjacent to the cloning site. Commercially available kits (e.g., the Sequenase® kit, United States Biochemical Corp., Cleveland, Ohio) are used to facilitate sequencing the DNA.

One of skill will appreciate that, utilizing the sequence information provided for the variable regions, nucleic acids encoding these sequences are obtained using a number of methods well known to those of skill in the art. Thus, DNA encoding the variable regions is prepared by any suitable method, including, for example, amplification techniques such as ligase chain reaction (LCR) (see, e.g., Wu & Wallace (1989) Genomics 4:560, Landegren, et al. (1988) Science 241:1077, and Barringer, et al. (1990) Gene 89:117), transcription amplification (see, e.g., Kwoh, et al. (1989) Proc. Natl. Acad. Sci. USA 86:1173), and self-sustained sequence replication (see, e.g., Guatelli, et al. (1990) Proc. Natl. Acad. Sci. USA 87:1874), cloning and restriction of appropriate sequences or direct chemical synthesis by methods such as the phosphotriester method of Narang, et al., (1979) Meth. Enzymol. 68:90; the phosphodiester method of Brown, et al., (1979) Meth. Enzymol. 68:109; the diethylphosphoramidite method of Beaucage, et al., (1981) Tetra. Lett. 22:1859; and the solid support method of U.S. Pat. No. 4,458,066.

The nucleic acid sequences that encode the single chain antibodies, or variable domains, are identified by techniques well known in the art (see, Sambrook, et al., supra). Briefly, the DNA products described above are separated on an electrophoretic gel. The contents of the gel are transferred to a suitable membrane (e.g., Hybond-N®, Amersham) and hybridized to a suitable probe under stringent conditions. The probe should comprise a nucleic acid sequence of a fragment embedded within the desired sequence.

If the DNA sequence is synthesized chemically, a single stranded oligonucleotide will result. This may be converted into double stranded DNA by hybridization with a complementary sequence, or by polymerization with a DNA polymerase using the single strand as a template. While it is possible to chemically synthesize an entire single chain Fv region, it is preferable to synthesize a number of shorter sequences (about 100 to 150 bases) that are later ligated together.

Alternatively, subsequences may be cloned and the appropriate subsequences cleaved using appropriate restriction enzymes. The fragments may then be ligated to produce the desired DNA sequence.

Nucleic acids encoding monoclonal antibodies or variable domains thereof are typically cloned into intermediate vectors before transformation into prokaryotic or eukaryotic cells for replication and/or expression. These intermediate vectors are typically prokaryote vectors, e.g., plasmids, or shuttle vectors. Isolated nucleic acids encoding therapeutic proteins comprise a nucleic acid sequence encoding a therapeutic protein and subsequences, interspecies homologues, alleles and polymorphic variants thereof.

To obtain high level expression of a cloned gene, such as those cDNAs encoding a suitable monoclonal antibody, one typically subclones the gene encoding the monoclonal antibody into an expression vector that contains a strong promoter to direct transcription, a transcription/translation terminator, and if for a nucleic acid encoding a protein, a ribosome binding site for translational initiation. Suitable promoters are well known in the art and described, e.g., in Sambrook et al., supra and Ausubel et al., supra. Eukaryotic expression systems for mammalian cells are well known in the art and are also commercially available. Kits for such expression systems are commercially available.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

The promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

The nucleic acid comprises a promoter to facilitate expression of the nucleic acid within a cell. Suitable promoters include strong, eukaryotic promoter such as, for example promoters from cytomegalovirus (CMV), mouse mammary tumor virus (MMTV), Rous sarcoma virus (RSV), and adenovirus. More specifically, suitable promoters include the promoter from the immediate early gene of human CMV (Boshart et al., (1985) Cell 41:521) and the promoter from the long terminal repeat (LTR) of RSV (Gorman et al., (1982) Proc. Natl. Acad. Sci. USA 79:6777).

For eukaryotic expression, the construct may comprise at a minimum a eukaryotic promoter operably linked to a nucleic acid operably linked to a polyadenylation sequence. The polyadenylation signal sequence may be selected from any of a variety of polyadenylation signal sequences known in the art, such as, for example, the SV40 early polyadenylation signal sequence. The construct may also include one or more introns, which can increase levels of expression of the nucleic acid of interest, particularly where the nucleic acid of interest is a cDNA (e.g., contains no introns of the naturally-occurring sequence). Any of a variety of introns known in the art may be used.

Other components of the construct may include, for example, a marker (e.g., an antibiotic resistance gene (such as an ampicillin resistance gene)) to aid in selection of cells containing and/or expressing the construct, an origin of replication for stable replication of the construct in a bacterial cell (preferably, a high copy number origin of replication), a nuclear localization signal, or other elements which facilitate production of the nucleic acid construct, the protein encoded thereby, or both.

In addition to the promoter, the expression vector typically contains a transcription unit or expression cassette that contains all the additional elements required for the expression of the nucleic acid in host cells. A typical expression cassette thus contains a promoter operably linked to the nucleic acid sequence and signals required for efficient polyadenylation of the transcript, ribosome binding sites, and translation termination. The nucleic acid sequence may typically be linked to a cleavable signal peptide sequence to promote secretion of the encoded protein by the transformed cell. Such signal peptides would include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette may also contain a transcription termination region downstream of the structural gene to provide for efficient termination. The termination region may be obtained from the same gene as the promoter sequence or may be obtained from different genes.

Some expression systems have markers that provide gene amplification such as thymidine kinase, hygromycin B phosphotransferase, and dihydrofolate reductase.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells.

Standard transfection methods are used to produce bacterial, mammalian, yeast, insect, or plant cell lines that express large quantities of the antibody or variable region domains, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the monoclonal antibody or a variable domain thereof.

After the expression vector is introduced into the cells, the transfected cells are cultured under conditions favoring expression of the monoclonal antibody or ariable domain region. The expressed protein is recovered from the culture using standard techniques known to those of skill in the art.

The monoclonal antibody or variable domain region may be purified to substantial purity by standard techniques known to those of skill in the art, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

c. Covalent Modifications of Polypeptide Variants

Covalent modifications of polypeptide variants are included within the scope of this invention. The modifications are made by chemical synthesis or by enzymatic or chemical cleavage or elaboration of the mutant antibody of the invention. Other types of covalent modifications of the polypeptide variant are introduced into the molecule by reacting targeted amino acid residues of the polypeptide variant with an organic derivatizing agent that is capable of reacting with selected side chains or the N- or C-terminal residues.

The modifications of the mutant antibody of the invention include the attachment of agents to, for example, enhance antibody stability, water-solubility, in vivo half-life and to target the antibody to a desired target tissue. Many methods are known in the art for derivatizing both the mutant antibodies of the invention. The discussion that follows is illustrative of reactive groups found on the mutant antibody and on the antigen and methods of forming conjugates between the mutant antibody and an antigen or ligand. The use of homo- and hetero-bifunctional derivatives of each of the reactive functionalities discussed below to link the mutant antibody to the antigen is within the scope of the present invention.

Cysteinyl residues most commonly are reacted with agents that include α-haloacetates (and corresponding amines), such as chloroacetic acid or chloroacetamide, to give carboxymethyl or carboxyamidomethyl derivatives. Cysteinyl residues also are derivatized by reaction with bromotrifluoroketones, α-bromo-β-(5-imidozoyl)carboxylic acids, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with, for example, groups that include pyrocarbonate at pH 5.5-7.0 because this agent is relatively specific for the histidyl side chain. Para-bromophenacyl halides also are useful; the reaction is preferably performed in 0.1 M sodium cacodylate at pH 6.0.

Lysinyl and amino-terminal residues are reacted with succinic or other carboxylic acid anhydrides. Derivatization with these agents has the effect of reversing the charge of the lysinyl residues. Other suitable reagents for derivatizing α-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKₐ of the guanidine site. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using ¹²⁵I or ¹³¹I to prepare labeled proteins for use in radioimmunoassay, the chloramine T method described above being suitable.

Carboxyl side groups (aspartyl or glutamyl) are selectively modified by reaction with carbodiimides (R—N═C═N—R′), where R and R′ are different alkyl groups, such as 1-cyclohexyl-3-(2-morpholinyl-4-ethyl)carbodiimide or 1-ethyl-3-(4-azo-4,4-dimethylpentyl)carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues, respectively. These residues are deamidated under neutral or basic conditions. The deamidated form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, PROTEINS: STRUCTURE AND MOLECULAR PROPERTIES, W. H. Freeman & Co., San Francisco, pp. 79-86 (1983)), acetylation of the N-terminal amine, and amidation of any C-terminal carboxyl group.

Another type of covalent modification of the polypeptide variant included within the scope of this invention comprises altering the original glycosylation pattern of the polypeptide variant. By altering is meant deleting one or more carbohydrate moieties found in the polypeptide variant, and/or adding one or more glycosylation sites that are not present in the polypeptide variant.

Glycosylation of the mutant antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the mutant antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original polypeptide variant (for O-linked glycosylation sites). For ease, the polypeptide variant amino acid sequence is preferably altered through changes at the DNA level, particularly by mutating the DNA encoding the polypeptide variant at preselected bases such that codons are generated that will translate into the desired amino acids. The DNA mutation(s) may be made using methods described above.

Another means of increasing the number of carbohydrate moieties on the mutant antibody is by chemical or enzymatic coupling of glycosides to the polypeptide variant. These procedures are advantageous in that they do not require production of the polypeptide variant in a host cell that has glycosylation capabilities for N- or O-linked glycosylation. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. These methods are described in WO 87/05330 published Sep. 11, 1987, and in Aplin and Wriston, CRC CRIT. REV. BIOCHEM., pp. 259-306 (1981).

Removal of any carbohydrate moieties present on the mutant antibody is accomplished either chemically or enzymatically. Chemical deglycosylation requires exposure of the polypeptide variant to the compound trifluoromethanesulfonic acid, or an equivalent compound. This treatment results in the cleavage of most or all sugars except the linking sugar (N-acetylglucosamine or N-acetylgalactosamine), while leaving the mutant antibody intact. Chemical deglycosylation is described by Hakimuddin et al., Arch. Biochem. Biophys. 259: 52 (1987) and by Edge et al., Anal. Biochem. 118: 131 (1981). Enzymatic cleavage of carbohydrate moieties on polypeptide variants can be achieved by the use of a variety of endo- and exo-glycosidases as described by Thotakura et al., Meth. Enzymol. 138: 350 (1987).

Another type of covalent modification of the polypeptide variant comprises linking the polypeptide variant to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or U.S. Pat. No. 4,179,337. The polymers may be added to alter the properties of the mutant antibody.

d. Preparation of the Mutant Antibody-Linker Moiety Conjugate

A variety of reagents are used to modify the components of the conjugate with intramolecular chemical crosslinks (for reviews of crosslinking reagents and crosslinking procedures see: Wold, F., Meth. Enzymol. 25: 623-651, 1972; Weetall, H. H., and Cooney, D. A., In: ENZYMES AS DRUGS. (J. S. Holcenberg, and J. Roberts, eds.) pp. 395-442, Wiley, New York, 1981; Ji, T. H., Meth. Enzymol. 91: 580-609, 1983; Mattson et al., Mol. Biol. Rep. 17: 167-183, 1993, all of which are incorporated herein by reference). Preferred useful crosslinking reagents are derived from various zero-length, homo-bifunctional, and hetero-bifunctional crosslinking reagents. Zero-length crosslinking reagents include direct conjugation of two intrinsic chemical groups with no introduction of extrinsic material. Agents that catalyze formation of a disulfide bond belong to this category. Another example is reagents that induce condensation of a carboxyl and a primary amino group to form an amide bond such as carbodiimides, ethylchloroformate, Woodward's reagent K (2-ethyl-5-phenylisoxazolium-3′-sulfonate), and carbonyldiimidazole. In addition to these chemical reagents, the enzyme transglutaminase (glutamyl-peptide γ-glutamyltransferase; EC 2.3.2.13) may be used as zero-length crosslinking reagent. This enzyme catalyzes acyl transfer reactions at carboxamide groups of protein-bound glutaminyl residues, usually with a primary amino group as substrate. Preferred homo- and hetero-bifunctional reagents contain two identical or two dissimilar sites, respectively, which may be reactive for amino, sulfhydryl, guanidino, indole, or nonspecific groups.

e. Preferred Specific Sites in Crosslinking Reagents

1. Amino-Reactive Groups

In one preferred embodiment, the sites are amino-reactive groups. Useful non-limiting examples of amino-reactive groups include N-hydroxysuccinimide (NHS) esters, imidoesters, isocyanates, acylhalides, arylazides, p-nitrophenyl esters, aldehydes, and sulfonyl chlorides.

NHS esters react preferentially with the primary (including aromatic) amino groups of the affinity component. The imidazole groups of histidines are known to compete with primary amines for reaction, but the reaction products are unstable and readily hydrolyzed. The reaction involves the nucleophilic attack of an amine on the acid carboxyl of an NHS ester to form an amide, releasing the N-hydroxysuccinimide. Thus, the positive charge of the original amino group is lost.

Imidoesters are the most specific acylating reagents for reaction with the amine groups of the conjugate components. At a pH between 7 and 10, imidoesters react only with primary amines. Primary amines attack imidates nucleophilically to produce an intermediate that breaks down to amidine at high pH or to a new imidate at low Ph. The new imidate can react with another primary amine, thus crosslinking two amino groups, a case of a putatively monofunctional imidate reacting bifunctionally. The principal product of reaction with primary amines is an amidine that is a stronger base than the original amine. The positive charge of the original amino group is therefore retained. As a result, imidoesters do not affect the overall charge of the conjugate.

Isocyanates (and isothiocyanates) react with the primary amines of the conjugate components to form stable bonds. Their reactions with sulfhydryl, imidazole, and tyrosyl groups give relatively unstable products.

Acylazides are also used as amino-specific reagents in which nucleophilic amines of the affinity component attack acidic carboxyl groups under slightly alkaline conditions, e.g. pH 8.5.

Arylhalides such as 1,5-difluoro-2,4-dinitrobenzene react preferentially with the amino groups and tyrosine phenolic groups of the conjugate components, but also with its sulfhydryl and imidazole groups.

p-Nitrophenyl esters of mono- and dicarboxylic acids are also useful amino-reactive groups. Although the reagent specificity is not very high, α- and ε-amino groups appear to react most rapidly.

Aldehydes such as glutaraldehyde react with primary amines of the conjugate components (e.g., ε-amino group of lysine residues). Glutaraldehyde, however, displays also reactivity with several other amino acid side chains including those of cysteine, histidine, and tyrosine. Since dilute glutaraldehyde solutions contain monomeric and a large number of polymeric forms (cyclic hemiacetal) of glutaraldehyde, the distance between two crosslinked groups within the affinity component varies. Although unstable Schiff bases are formed upon reaction of the protein amino groups with the aldehydes of the polymer, glutaraldehyde is capable of modifying the affinity component with stable crosslinks. At pH 6-8, the pH of typical crosslinking conditions, the cyclic polymers undergo a dehydration to form α-β unsaturated aldehyde polymers. Schiff bases, however, are stable, when conjugated to another double bond. The resonant interaction of both double bonds prevents hydrolysis of the Schiff linkage. Furthermore, amines at high local concentrations can attack the ethylenic double bond to form a stable Michael addition product.

Aromatic sulfonyl chlorides react with a variety of sites of the conjugate components, but reaction with the amino groups is the most important, resulting in a stable sulfonamide linkage.

2. Sulfhydryl-Reactive Groups

In another preferred embodiment, the sites are sulfhydryl-reactive groups. Useful non-limiting examples of sulfhydryl-reactive groups include maleimides, alkyl halides, pyridyl disulfides, thiophthalimides, and Michael acceptors e.g., acrylamides.

Maleimides react preferentially with the sulfhydryl group of the conjugate components to form stable thioether bonds. They also react at a much slower rate with primary amino groups and the imidazole groups of histidines. However, at pH 7 the maleimide group can be considered a sulfhydryl-specific group, since at this pH the reaction rate of simple thiols is 1000-fold greater than that of the corresponding amine.

Alkyl halides react with sulfhydryl groups, sulfides, imidazoles, and amino groups. At neutral to slightly alkaline pH, however, alkyl halides react primarily with sulfhydryl groups to form stable thioether bonds. At higher pH, reaction with amino groups is favored.

Pyridyl disulfides react with free sulfhydryls via disulfide exchange to give mixed disulfides. As a result, pyridyl disulfides are the most specific sulfhydryl-reactive groups.

Thiophthalimides react with free sulfhydryl groups to form also disulfides.

3. Guanidino-Reactive Groups

In another embodiment, the sites are guanidino-reactive groups. A useful non-limiting example of a guanidino-reactive group is phenylglyoxal. Phenylglyoxal reacts primarily with the guanidino groups of arginine residues in the affinity component. Histidine and cysteine also react, but to a much lesser extent.

4. Indole-Reactive Groups

In another embodiment, the sites are indole-reactive groups. Useful non-limiting examples of indole-reactive groups are sulfenyl halides. Sulfenyl halides react with tryptophan and cysteine, producing a thioester and a disulfide, respectively. To a minor extent, methionine may undergo oxidation in the presence of sulfenyl chloride.

5. Carboxyl-Reactive Residue

In another embodiment, carbodiimides soluble in both water and organic solvent, are used as carboxyl-reactive reagents. These compounds react with free carboxyl groups forming a pseudourea that can then couple to available amines yielding an amide linkage (Yamada et al., Biochemistry 20: 4836-4842, 1981) teach how to modify a protein with carbodiimde.

f. Preferred Nonspecific Sites in Crosslinking Reagents

In addition to the use of site-specific reactive moieties, the present invention contemplates the use of non-specific reactive groups as cross-linking agents. Non-specific groups include photoactivatable groups, for example. In another preferred embodiment, the sites are photoactivatable groups. Photoactivatable groups, completely inert in the dark, are converted to reactive species upon absorption of a photon of appropriate energy. In one preferred embodiment, photoactivatable groups are selected from precursors of nitrenes generated upon heating or photolysis of azides. Electron-deficient nitrenes are extremely reactive and can react with a variety of chemical bonds including N—H, O—H, C—H, and C═C. Although three types of azides (aryl, alkyl, and acyl derivatives) may be employed, arylazides are presently preferrred. The reactivity of arylazides upon photolysis is better with N—H and O—H than C—H bonds. Electron-deficient arylnitrenes rapidly ring-expand to form dehydroazepines, which tend to react with nucleophiles, rather than form C—H insertion products. The reactivity of arylazides can be increased by the presence of electron-withdrawing substituents such as nitro or hydroxyl groups in the ring. Such substituents push the absorption maximum of arylazides to longer wavelength. Unsubstituted arylazides have an absorption maximum in the range of 260-280 nm, while hydroxy and nitroarylazides absorb significant light beyond 305 nm. Therefore, hydroxy and nitroarylazides are most preferable since they allow to employ less harmful photolysis conditions for the affinity component than unsubstituted arylazides.

In another preferred embodiment, photoactivatable groups are selected from fluorinated arylazides. The photolysis products of fluorinated arylazides are arylnitrenes, all of which undergo the characteristic reactions of this group, including C—H bond insertion, with high efficiency (Keana et al., J. Org. Chem. 55: 3640-3647, 1990).

In another embodiment, photoactivatable groups are selected from benzophenone residues. Benzophenone reagents generally give higher crosslinking yields than arylazide reagents.

In another embodiment, photoactivatable groups are selected from diazo compounds, which form an electron-deficient carbene upon photolysis. These carbenes undergo a variety of reactions including insertion into C—H bonds, addition to double bonds (including aromatic systems), hydrogen attraction and coordination to nucleophilic centers to give carbon ions.

In still another embodiment, photoactivatable groups are selected from diazopyruvates. For example, the p-nitrophenyl ester of p-nitrophenyl diazopyruvate reacts with aliphatic amines to give diazopyruvic acid amides that undergo ultraviolet photolysis to form aldehydes. The photolyzed diazopyruvate-modified affinity component will react like formaldehyde or glutaraldehyde forming intraprotein crosslinks.

g. Homobifunctional Reagents

1. Homobifunctional Crosslinkers Reactive with Primary Amines

Synthesis, properties, and applications of such reagents are described in the literature (for reviews of crosslinking procedures and reagents, see above). Many reagents are available (e.g., Pierce Chemical Company, Rockford, Ill.; Sigma Chemical Company, St. Louis, Mo.; Molecular Probes, Inc., Eugene, Oreg.).

Preferred, non-limiting examples of homobifunctional NHS esters include disuccinimidyl glutarate (DSG), disuccinimidyl suberate (DSS), bis(sulfosuccinimidyl) suberate (BS), disuccinimidyl tartarate (DST), disulfosuccinimidyl tartarate (sulfo-DST), bis-2-(succinimidooxycarbonyloxy)ethylsulfone (BSOCOES), bis-2-(sulfosuccinimidooxy-carbonyloxy)ethylsulfone (sulfo-BSOCOES), ethylene glycolbis(succinimidylsuccinate) (EGS), ethylene glycolbis(sulfosuccinimidylsuccinate) (sulfo-EGS), dithiobis(succinimidyl-propionate (DSP), and dithiobis(sulfosuccinimidylpropionate (sulfo-DSP). Preferred, non-limiting examples of homobifunctional imidoesters include dimethyl malonimidate (DMM), dimethyl succinimidate (DMSC), dimethyl adipimidate (DMA), dimethyl pimelimidate (DMP), dimethyl suberimidate (DMS), dimethyl-3,3′-oxydipropionimidate (DODP), dimethyl-3,3′-(methylenedioxy)dipropionimidate (DMDP), dimethyl-,3′-(dimethylenedioxy)dipropionimidate (DDDP), dimethyl-3,3′-(tetramethylenedioxy)-dipropionimidate (DTDP), and dimethyl-3,3′-dithiobispropionimidate (DTBP).

Preferred, non-limiting examples of homobifunctional isothiocyanates include: p-phenylenediisothiocyanate (DITC), and 4,4′-diisothiocyano-2,2′-disulfonic acid stilbene (DIDS).

Preferred, non-limiting examples of homobifunctional isocyanates include xylene-diisocyanate, toluene-2,4-diisocyanate, toluene-2-isocyanate-4-isothiocyanate, 3-methoxydiphenylmethane-4,4′-diisocyanate, 2,2′-dicarboxy-4,4′-azophenyldiisocyanate, and hexamethylenediisocyanate.

Preferred, non-limiting examples of homobifunctional arylhalides include 1,5-difluoro-2,4-dinitrobenzene (DFDNB), and 4,4′-difluoro-3,3′-dinitrophenyl-sulfone.

Preferred, non-limiting examples of homobifunctional aliphatic aldehyde reagents include glyoxal, malondialdehyde, and glutaraldehyde.

Preferred, non-limiting examples of homobifunctional acylating reagents include nitrophenyl esters of dicarboxylic acids.

Preferred, non-limiting examples of homobifunctional aromatic sulfonyl chlorides include phenol-2,4-disulfonyl chloride, and .alpha.-naphthol-2,4-disulfonyl chloride.

Preferred, non-limiting examples of additional amino-reactive homobifunctional reagents include erythritolbiscarbonate which reacts with amines to give biscarbamates.

2. Homobifunctional Crosslinkers Reactive with Free Sulfhydryl Groups

Synthesis, properties, and applications of such reagents are described in the literature (for reviews of crosslinking procedures and reagents, see above). Many of the reagents e.g., Michael acceptors such as acrylamides, are commercially available (e.g., Pierce Chemical Company, Rockford, Ill.; Sigma Chemical Company, St. Louis, Mo.; Molecular Probes, Inc., Eugene, Oreg.).

Preferred, non-limiting examples of homobifunctional maleimides include bismaleimidohexane (BMH), N,N′-(1,3-phenylene) bismaleimide, N,N′-(1,2-phenylene)bismaleimide, azophenyldimaleimide, and bis(N-maleimidomethyl)ether. Preferred, non-limiting examples of homobifunctional pyridyl disulfides include 1,4-di->3′-(2′-pyridyldithio)propionamidobutane (DPDPB).

Preferred, non-limiting examples of homobifunctional alkyl halides include 2,2′-dicarboxy-4,4′-diiodoacetamidoazobenzene, α,α′-diiodo-p-xylenesulfonic acid, α,α′-dibromo-1p-xylenesulfonic acid, N,N′-bis(b-bromoethyl)benzylamine, N,N′-di(bromoacetyl)phenylthydrazine, and 1,2-di(bromoacetyl)amino-3-phenylpropane.

3. Homobifunctional Photoactivatable Crosslinkers

Synthesis, properties, and applications of such reagents are described in the literature (for reviews of crosslinking procedures and reagents, see above). Some of the reagents are commercially available (e.g., Pierce Chemical Company, Rockford, Ill.; Sigma Chemical Company, St. Louis, Mo.; Molecular Probes, Inc., Eugene, Oreg.).

Preferred, non-limiting examples of homobifunctional photoactivatable crosslinker include bis-b-(4-azidosalicylamido)ethyldisulfide (BASED), di-N-(2-nitro-4-azidophenyl)-cystamine-S,S-dioxide (DNCO), and 4,4′-dithiobisphenylazide.

h. Hetero-Bifunctional Reagents

1. Amino-Reactive Hetero-Bifunctional Reagents with a Pyridyl Disulfide Moiety

Synthesis, properties, and applications of such reagents are described in the literature (for reviews of crosslinking procedures and reagents, see above). Many of the reagents are commercially available (e.g., Pierce Chemical Company, Rockford, Ill.; Sigma Chemical Company, St. Louis, Mo.; Molecular Probes, Inc., Eugene, Oreg.).

Preferred, non-limiting examples of hetero-bifunctional reagents with a pyridyl disulfide moiety and an amino-reactive NHS ester include N-succinimidyl-3-(2-pyridyldithio)propionate (SPDP), succinimidyl 6-3-(2-pyridyldithio)propionamidohexanoate (LC-SPDP), sulfosuccinimidyl 6-3-(2-pyridyldithio)propionamidohexanoate (sulfo-LCSPDP), 4-succinimidyloxycarbonyl-a-methyl-α-(2-pyridyldithio)toluene (SMPT), and sulfosuccinimidyl 6-a-methyl-α-(2-pyridyldithio)toluamidohexanoate (sulfo-LC-SMPT).

2. Amino-Reactive Hetero-Bifunctional Reagents with a Maleimide Moiety

Synthesis, properties, and applications of such reagents are described in the literature. Preferred, non-limiting examples of hetero-bifunctional reagents with a maleimide moiety and an amino-reactive NHS ester include succinimidyl maleimidylacetate (AMAS), succinimidyl 3-maleimidylpropionate (BMPS), N-γ-maleimidobutyryloxysuccinimide ester (GMBS)N-γ-maleimidobutyryloxysulfo succinimide ester (sulfo-GMBS) succinimidyl 6-maleimidylhexanoate (EMCS), succinimidyl 3-maleimidylbenzoate (SMB), m-maleimidobenzoyl-N-hydroxysuccinimide ester (MBS), m-maleimidobenzoyl-N-hydroxysulfosuccinimide ester (sulfo-MBS), succinimidyl 4-(N-maleimidomethyl)-1cyclohexane-1-carboxylate (SMCC), sulfosuccinimidyl 4-(N-maleimidomethyl)cyclohexane-1-carboxylate (sulfo-SMCC), succinimidyl 4-(p-maleimidophenyl)butyrate (SMPB), and sulfosuccinimidyl 4-(p-maleimidophenyl)butyrate (sulfo-SMPB).

3. Amino-Reactive Hetero-Bifunctional Reagents with an Alkyl Halide Moiety

Synthesis, properties, and applications of such reagents are described in the literature. Preferred, non-limiting examples of hetero-bifunctional reagents with an alkyl halide moiety and an amino-reactive NHS ester include N-succinimidyl-(4-iodoacetyl)aminobenzoate (SIAB), sulfosuccinimidyl-(4-iodoacetyl)aminobenzoate (sulfo-SIAB), succinimidyl-6-(iodoacetyl)aminohexanoate (SIAX), succinimidyl-6-(6-((iodoacetyl)-amino)hexanoylamino)hexanoate (SIAXX), succinimidyl-6-(((4-(iodoacetyl)-amino)-methyl)-cyclohexane-1-carbonyl)aminohexanoate (SIACX), and succinimidyl-4-((iodoacetyl)-amino)methylcyclohexane-1-carboxylate (SIAC).

A preferred example of a hetero-bifunctional reagent with an amino-reactive NHS ester and an alkyl dihalide moiety is N-hydroxysuccinimidyl 2,3-dibromopropionate (SDBP). SDBP introduces intramolecular crosslinks to the affinity component by conjugating its amino groups. The reactivity of the dibromopropionyl moiety for primary amino groups is controlled by the reaction temperature (McKenzie et al., Protein Chem. 7: 581-592 (1988)).

Preferred, non-limiting examples of hetero-bifunctional reagents with an alkyl halide moiety and an amino-reactive p-nitrophenyl ester moiety include p-nitrophenyl iodoacetate (NPIA) and Michael acceptors e.g., acrylamides.

4. Photoactivatable Arylazide-Containing Hetero-Bifunctional Reagents with a NHS Ester Moiety

Synthesis, properties, and applications of such reagents are described in the literature. Preferred, non-limiting examples of photoactivatable arylazide-containing hetero-bifunctional reagents with an amino-reactive NHS ester include N-hydroxysuccinimidyl-4-azidosalicylic acid (NHS-ASA), N-hydroxysulfosuccinimidyl-4-azidosalicylic acid (sulfo-NHS-ASA), sulfosuccinimidyl-(4-azidosalicylamido)hexanoate (sulfo-NHS-LC-ASA), N-hydroxysuccinimidyl N-(4-azidosalicyl)-6-aminocaproic acid (NHS-ASC), N-hydroxy-succinimidyl-4-azidobenzoate (HSAB), N-hydroxysulfo-succinimidyl-4-azidobenzoate (sulfo-HSAB), sulfosuccinimidyl-4-(p-azidophenyl)butyrate (sulfo-SAPB), N-5-azido-2-nitrobenzoyloxy-succinimide (ANB-NOS), N-succinimidyl-6-(4′-azido-2′-nitrophenyl-amino)hexanoate (SANPAH), sulfosuccinimidyl-6-(4′-azido-2′-nitrophenylamino)-hexanoate (sulfo-SANPAH), N-succinimidyl 2-(4-azidophenyl)dithioacetic acid (NHS-APDA), N-succinimidyl-(4-azidophenyl)1,3′-dithiopropionate (SADP), sulfosuccinimidyl-(4-azidophenyl)-1,3′-dithiopropionate (sulfo-SADP), sulfosuccinimidyl-2-(m-azido-o-nitrobenzamido)ethyl-1,3′-dithiopropionate (SAND), sulfosuccinimidyl-2-(p-azidosalicylamido)-ethyl-1,3′-dithiopropionate (SASD), N-hydroxysuccinimidyl 4-azidobenzoylglycyltyrosine (NHS-ABGT), sulfosuccinimidyl-2-(7-azido-4-4-methylcoumarin-3-acetamide)ethyl-1,3′-dithiopropionate (SAED), and sulfosuccinimidyl-7-azido-4-methylcoumarin-3-acetate (sulfo-SAMCA).

Other cross-linking agents are known to those of skill in the art (see, for example, Pomato et al., U.S. Pat. No. 5,965,106.

i. Fusion Proteins

In a preferred form, the antibodies are recombinantly produced as fusion proteins, to form bispecific antibodies that bind to an antigen of a targeted tumor and a metal chelate.

Dozens of antitumor antigens and antibodies against them are known in the art, many of which are in clinical trials. Examples include AMD-Fab, LDP-02, αCD-11a, αCD-18, α-VEGF, α-IgE, and Herceptin, from Genentech, ABX-CBL, ABX-EGF, and ABX-IL8, from Abgenix, and aCD3, Smart 195 and Zenepax from Protein Design Labs. In preferred forms, the antibody is HMFG1, L6, or Lym-1, with Lym-1 being the most preferred. In preferred embodiments, an scFv or dsFv form of the antibody is employed. Formation of scFvs and dsFvs is known in the art. Formation of a scFv of Lym-1, for example, is described in Bin Song et al., Biotechnol Appl Biochem 28(2):163-7 (1998). See, also Cancer Immunol. Immunother. 43: 26-30 (1996). The two antibodies can be linked directly or, more commonly, are connected by a short peptide linker, such as Gly₄Ser repeated 3 times.

2. The Chelates

In addition to the mutant antibodies described in detail above, the invention also provides reactive chelates that are specifically recognized by an antibody antigen recognition domain (CDR) and which form a covalent bond with the reactive group on the mutant antibody.

In an exemplary embodiment, there is provided a metal chelate having four nitrogen atoms that is recognized by the antigen recognition domain of a mutant antibody. The antibody includes a reactive site not present in the wildtype of the antibody and the reactive site is in a position proximate to or within the antigen recognition domain.

In a preferred embodiment, the chelate includes a substituted or unsubstituted ethyl bridge that covalently links at least two of the nitrogen atoms. An exemplary ethyl bridge is shown in the formula below: 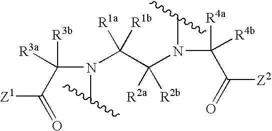
wherein Z¹ and Z² are members independently selected from OR and NR³R⁴, in which R³ and R⁴ are members independently selected from H, substituted or unsubstituted alkyl and substituted or unsubstituted heteroalkyl. The symbols R^{1a}, R^{1b}, R^{2a}, R^{2b}, R^{3a}, R^{3b}, R^{4a} and R^{4b} represent members independently selected from H, substituted or unsubstituted alkyl, substituted or unsubstituted heteroalkyl, substituted or unsubstituted aryl and linker moieties.

In another exemplary embodiment, the chelate has the formula: 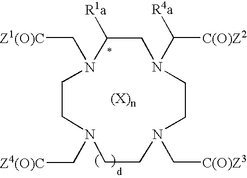
wherein Z¹, Z², Z³ and Z⁴ are members independently selected from OR¹ and NR¹R², in which R¹ and R² are members independently selected from H, substituted or unsubstituted alkyl and substituted or unsubstituted heteroalkyl. The symbol X represents a member selected from a lanthanide, an actinide, an alkaline earth metal, a group IIIb transition metal, or a metal. The symbol n represents 0 or 1; and d is 1 or 2. In a preferred embodiment, the carbon atom marked * is of S configuration.

In another exemplary embodiment, the chelate includes a moiety having the formula: 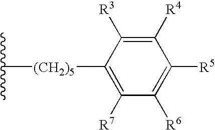
wherein s is 1-10, wherein R³, R⁴, R⁵, R⁶ and R⁷ are members independently selected from H, halogen, NO₂, CN, X¹R⁸, NR⁹R¹⁰, and C(X²)R¹¹. The symbol X¹ represents a member selected from O, NH and S. The symbols R⁸ and R⁹ are members independently selected from H, substituted or unsubstituted alkyl, substituted or unsubstituted heteroalkyl and C(Z³)R¹², in which X³ is a member selected from O, S and NH. R¹² is a member selected from substituted or unsubstituted alkyl, substituted or unsubstituted heteroalkyl and OR³, in which R¹³ is a member selected from substituted or unsubstituted alkyl, substituted or unsubstituted heteroalkyl, substituted or unsubstituted aryl and substituted or unsubstituted heteroaryl. The symbol R¹⁰ is a member selected from H, substituted or unsubstituted alkyl, substituted or unsubstituted heteroalkyl and OH, and R⁹ and R¹⁰, taken together are optionally (═C═S). X² is a member selected from O, S and NH. In some embodiments, R¹⁰ is —C(O)—CHCH². The symbol R¹¹ represents a member selected from H, halogen, substituted or unsubstituted alkyl, substituted or unsubstituted heteroalkyl, OR¹⁴, NR¹⁵R¹⁶ R¹⁴ is a member selected from H, substituted or unsubstituted alkyl, substituted or unsubstituted heteroalkyl, and C(O)R¹⁷. R¹⁷ is a member selected from substituted or unsubstituted alkyl and substituted or unsubstituted heteroalkyl; and R¹⁵ and R¹⁶ are members independently selected from H, substituted or unsubstituted alkyl and substituted or unsubstituted heteroalkyl.

In an exemplary embodiment, the chelate is (S)-2-(4-acrylamidobenzyl)-DOTA (AABD) and has the following formula:

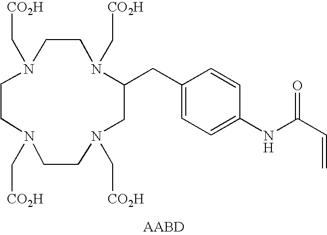

In practicing the present invention, the structure of the metal binding portion of the chelate is selected from an array of structures known to complex metal ions. Exemplary chelating agents of use in the present invention include, but are not limited to, reactive chelating groups capable of chelating radionuclides include macrocycles, linear, or branched moieties. Examples of macrocyclic chelating moieties include polyaza- and polyoxamacrocycles, polyether macrocycles, crown ether macrocycles, and cryptands (see, e.g., Synthesis of Macrocycles: the Diesgn of Selective Complexing Agents (Izatt and Christensen ed., 1987) and The Chemistry of Macrocyclic Ligand Complexes (Lindoy, 1989)). Examples of polyazamacrocyclic moieties include those derived from compounds such as 1,4,7,10-tetraazacyclododecane-N,N′,N″,N′″-tetraacetic acid (“DOTA”); 1,4,7,10-tetraazacyclotridecane-N,N′,N″,N′″-tetraacetic acid (“TRITA”); 1,4,8,11-tetraazacyclotetradecane-N,N′,N″,N′″-tetraacetic acid (“TETA”); and 1,5,9,13-tetraazacyclohexadecane-N,N′,N″,N′″-tetraacetic acid (abbreviated herein abbreviated as HETA). In a presently preferred embodiment, the chelating agent includes four nitrogen atoms. Other embodiments in which the chelate includes oxygen atoms or mixtures of oxygen and nitrogen atoms are within the scope of the present invention. Additional embodiments in which the chelate include three nitrogen atoms (e.g., 1,4,7-triazacyclononane-N,N′,N″ triacetic acid (NOTA) as described in, e.g., Studer and Meares, Bioconjugate Chemistry 3:337-341 (1992)) are also within the scope of the present invention.

Chelating moieties having carboxylic acid groups, such as DOTA, TRITA, HETA, and HEXA, may be derivatized to convert one or more carboxylic acid groups to reactive groups. Alternatively, a methylene group adjacent to an amine or a carboxylic acid group can be derivatized with a reactive functional group. Additional exemplary chelates of use in the present invention are set forth in Meares et al., U.S. Pat. No. 5,958,374.

The preparation of chelates useful in practicing the present invention is accomplished using art-recognized methodologies or modifications thereof. In a preferred embodiment of the invention, a reactive derivative of DOTA is used. Preparation of DOTA is described in, e.g., Moi et al., J. Am. Chem. Soc. 110:6266-67 (1988) and Renn and Meares, Bioconjugate Chem. 3:563-69 (1992). See also commonly owned and assigned U.S. Patent Publication No. 2004/0146934.

The chelate that is linked to the antibody or growth factor targeting agent will, of course, depend on the ultimate application of the invention. Where the aim is to provide an image of the tumor, one will desire to use a diagnostic agent that is detectable upon imaging, such as a paramagnetic, radioactive or fluorogenic agent. Many diagnostic agents are known in the art to be useful for imaging purposes, as are methods for their attachment to antibodies (see, e.g., U.S. Pat. Nos. 5,021,236 and 4,472,509, both incorporated herein by reference). In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred. Ions useful in other contexts, such as X-ray imaging, include but are not limited to lanthanum (III), gold (III), lead (II), and especially bismuth (III). Moreover, in the case of radioactive isotopes for therapeutic and/or diagnostic application, presently preferred isotopes include iodine¹³¹, iodine¹²³, technicium^{99m}, indium¹¹¹, rhenium¹⁸⁸, rhenium¹⁸⁶, gallium⁶⁷, copper⁶⁷, yttrium⁹⁰, iodine¹²⁵ or astatine²¹¹.

Antibody-Chelate Bond Formation

In general, after the formation of the antibody-antigen complex, the reactive chelate is administered. The chelate reactive functional group(s), is located at any position on the metal chelate. Reactive groups and classes of reactions useful in practicing the present invention are generally those that are well known in the art of bioconjugate chemistry. Currently favored classes of reactions available with reactive chelates are those that proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, Advanced Organic Chemistry, 3rd Ed., John Wiley & Sons, New York, 1985; Hermanson, Bioconjugate Techniques, Academic Press, San Diego, 1996; and Feeney et al., Modification of Proteins; Advances in Chemistry Series, Vol. 198, American Chemical Society, Washington, D.C., 1982.

Useful reactive pendant functional groups include, for example:
(a) carboxyl groups and various derivatives thereof including, but not limited to, N-hydroxysuccinimide esters, N-hydroxybenztriazole esters, acid halides (e.g., 1, Br, Cl), acyl imidazoles, thioesters, p-nitrophenyl esters, alkyl, alkenyl, alkynyl and aromatic esters;
(b) hydroxyl groups, which can be converted to, e.g., esters, ethers, aldehydes, etc.

(c) haloalkyl groups, wherein the halide can be later displaced with a nucleophilic group such as, for example, an amine, a carboxylate anion, thiol anion, carbanion, or an alkoxide ion, thereby resulting in the covalent attachment of a new group at the functional group of the halogen atom;
(d) dienophile groups, which are capable of participating in Diels-Alder reactions such as, for example, maleimido groups;
(e) aldehyde or ketone groups, such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be, for example, converted to disulfides or reacted with acyl halides;
(h) amine or sulfhydryl groups, which can be, for example, acylated, alkylated or oxidized;
(i) alkenes, which can undergo, for example, cycloadditions, acylation, Michael addition, etc;
(j) epoxides, which can react with, for example, amines and hydroxyl compounds; and
(k) phosphoramidites and other standard functional groups useful in nucleic acid synthesis.

The reactive functional groups can be chosen such that they do not participate in, or interfere with, the reactions necessary to assemble the reactive chelates. Alternatively, a reactive functional group can be protected from participating in the reaction by the presence of a protecting group. Those of skill in the art understand how to protect a particular functional group such that it does not interfere with a chosen set of reaction conditions. For examples of useful protecting groups, see, for example, Greene et al., PROTECTIVE GROUPS IN ORGANIC SYNTHESIS, John Wiley & Sons, New York, 1991.

B. The Methods

In addition to the compositions of the invention, in another aspect, there is also provided methods of using the compositions of the invention to treat a patient for a disease or condition or to diagnose the disease or condition. Thus, in one aspect, the invention provides a method of using the compositions of the invention to treat a patient for a disease or condition (e.g., cancer or autoimmune diseases, such as diabetes, arthritis, systemic lupus erythematosus) or to diagnose a condition or disease. The method comprising the steps of: (a) administering to the patient a mutant antibody comprising; a mutant polypeptide sequence, comprising a mutant amino acid at a position within or proximate to a complementarity determining region of the antibody, wherein the mutant amino acid is not present at that position in the wild type antibody, and a linker covalently bound to the mutant amino acid, the linker further comprising a reactive functional group. Subsequent to the antibody finding its specific antigen and binding, the reactive functional group forms a covalent bond with a group of complementary reactivity on said antigen, thereby forming an antigen-antibody complex with infinite binding affinity.

In some embodiments, the antibody administered to the patient is a bispecific antibody. In those embodiments where the antibody is a bispecific antibody that includes a metal chelate binding domain, a metal chelate may be administered to the patient after the antibody-antigen complex of infinite binding affinity has formed. Thus, in another aspect, the present invention provides a method in which the tissue is pretargeted with the antibodies described herein. Subsequently, the antibodies are reacted with a metal chelate to form a covalent bond between the antibody and the metal chelate thereby forming a complex with infinite binding affinity.

The bispecific antibody may include a domain from an antibody raised against essentially any chelate of any metal ion. In a preferred embodiment, the antibody is 2D12.5, a monoclonal antibody that binds metal chelates of DOTA and similar structures.

In an exemplary method the tissue is pretargeted with an antibody of the invention. In some embodiments, the antibody comprises a CDR that binds specifically with a component on the surface of a cell, and a CDR that binds a metal chelate, thereby forming a complex between the cells and the antibody. After the antibody has localized in the desired tissue, a metal chelate is administered to the patient. The chelate specifically binds to the antibody of the invention, forming an antibody-metal chelate complex.

In an exemplary embodiment the metal chelate and its antibody form an infinitely bound complex. The mutant antibody comprises: (i) an antigen recognition domain that specifically binds to the metal chelate; (ii) a reactive site not present in the wild-type of the antibody (the reactive site is in a position proximate to or within the antigen recognition domain); and (iii) a recognition moiety that binds specifically with the pretargeting reagent, thereby forming a complex between the pretargeting reagent and the mutant antibody. After the pretargeting reagent has localized in the desired tissue, following step (b), a metal chelate is administered to the patient. The chelate specifically binds to the mutant antibody of the invention, forming an antibody-antigen complex. Moreover, the chelate comprises a reactive functional group having a reactivity that is preferably complementary to the reactivity of the reactive site on the mutant antibody such that a covalent bond is formed via reaction of the reactive functional group of the chelate and the reactive site of the mutant antibody. After the antibody-antigen complex is formed, the reactive site of the antibody and that of the metal chelate react to form a covalent bond between the mutant antibody and the metal chelate.

Pretargeting methods have been developed to increase the target:background ratios of the detection or therapeutic agents. Examples of pre-targeting and biotin/avidin approaches are described, for example, in Goodwin et al., U.S. Pat. No. 4,863,713; Goodwin et al., J. Nucl. Med. 29: 226 (1988); Hnatowich et al., J. Nucl. Med. 28: 1294 (1987); Oehr et al., J. Nucl. Med. 29: 728 (1988); Klibanov et al., J. Nucl. Med. 29: 1951 (1988); Sinitsyn et al., J. Nucl. Med. 30: 66 (1989); Kalofonos et al., J. Nucl. Med. 31: 1791 (1990); Schechter et al., Int. J. Cancer 48:167 (1991); Paganelli et al., Cancer Res. 51:5960 (1991); Paganelli et al., Nucl. Med. Commun. 12: 211 (1991); Stickney et al., Cancer Res. 51: 6650 (1991); and Yuan et al., Cancer Res. 51:3119, 1991; all of which are incorporated by reference herein in their entirety.

In both of the above-described aspects of the invention, it is preferable that a significant proportion of the antibodies used remain on the cell surface to be accessible to a later introduced moiety containing the radioactive agent. Thus, it is generally preferable to choose antigens that are not rapidly endocytosed or otherwise internalized by the cell upon antibody binding. Preferably, at least one-quarter of the bound antibody should remain on the cell surface and not internalized. In some cases, however, even less of the bound antibody may remain on the cell surface. For example, for a particular tumor type, an antigen which has a high rate of internalization may still be used for pretargeting if there is no known antigen with a lower internalization rate (or for which an antibody is available) with which to image tumor locations. The suitability of a particular antigen can be determined by simple assays known in the art.

### EXAMPLES

### Example 1

### Combining Antibody Specificity and Permanent Binding with Reference to the Crystal Structure of the Antibody-Ligand Complex

The following example illustrates the practicallity of combining antibody specificity and permanent binding. In this example, the available crystal structure of the antibody-ligand complex (Love, R. et al. Biochemistry 32, 10950-10959 (1993)) was used to facilitate the design of mutants. The antibody is a site-directed Cys mutant, made by conventional techniques, and is stable for weeks at 4° C. The ligand was selected empirically. The antibody-ligand attachment occurs efficiently in complex physiological media, making this approach to antibody-ligand systems with infinite affinity easily suitable for broad application.

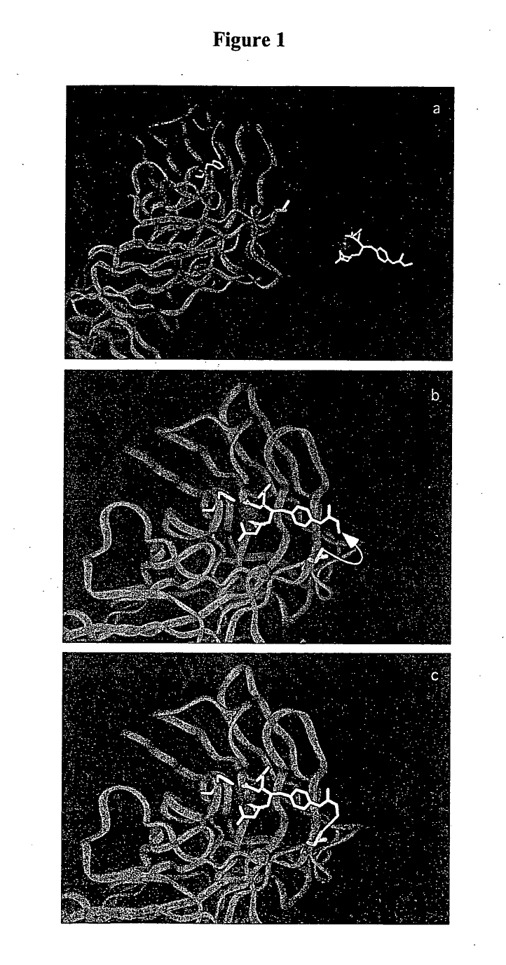

Preparation of antibody-ligand pairs that possess the binding specificity of antibodies, but do not dissociate is achieved by taking advantage of the slow dissociation of the correct ligand from the antibody combining site. The slow dissacociation allows sufficient time for the a permanent covalent bond to form during the lifetime of the complex (FIG. 1).

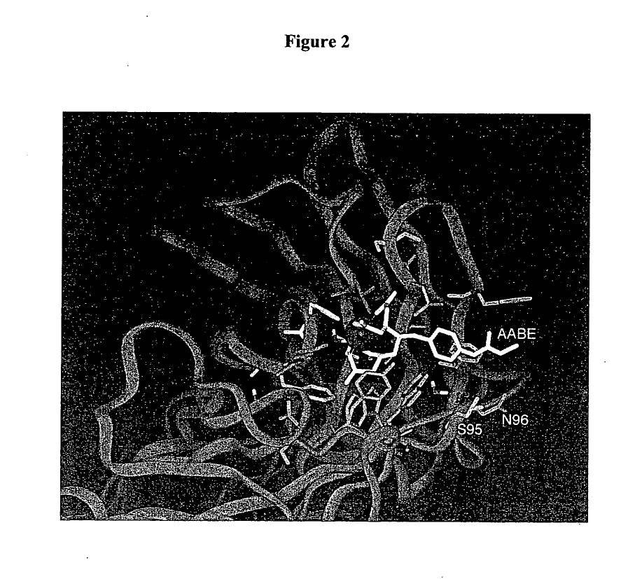

Using the gene for CHA255 and the crystal structure of the antibody-ligand complex (Love, R., et al., supra) we engineered chemically reactive sites near the ligand-binding site of the antibody by substituting cysteine at either position 95 or 96 of the light chain (FIG. 2). These residues were chosen because their side chains (i) are not exposed on the outer surface of the antibody, (ii) do not have any direct contacts with the bound ligand, and (iii) lie within a few angstroms of the para substituent of the ligand in the complex. Thus we expected to produce stable mutant proteins that retain the binding selectivity of the antibody. Cysteine was chosen because of its nucleophilicity, which often makes a free Cys sidechain the most reactive site on a protein. Because it was not exposed on the outer surface of the antibody, but rather was protected as part of the binding site, the chosen position in complementarity determining region 3 of the light chain moderated the reactivity of the Cys sidechain.

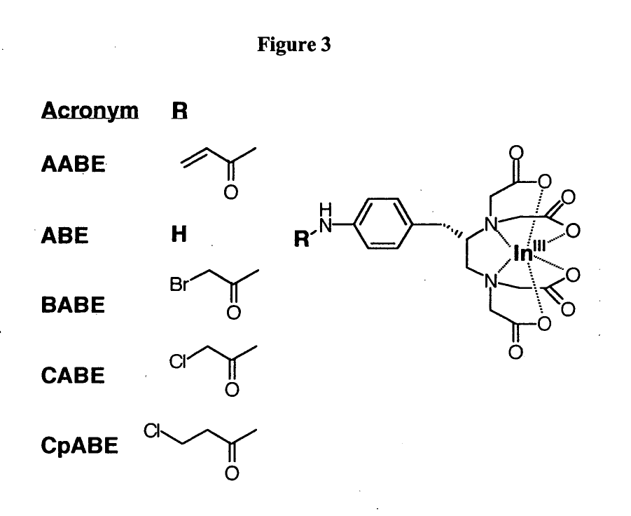

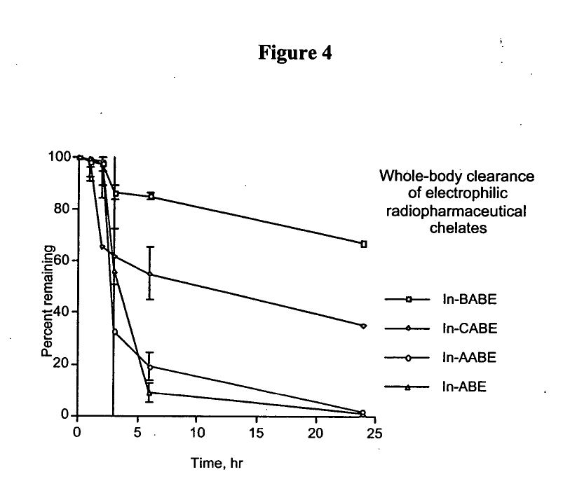

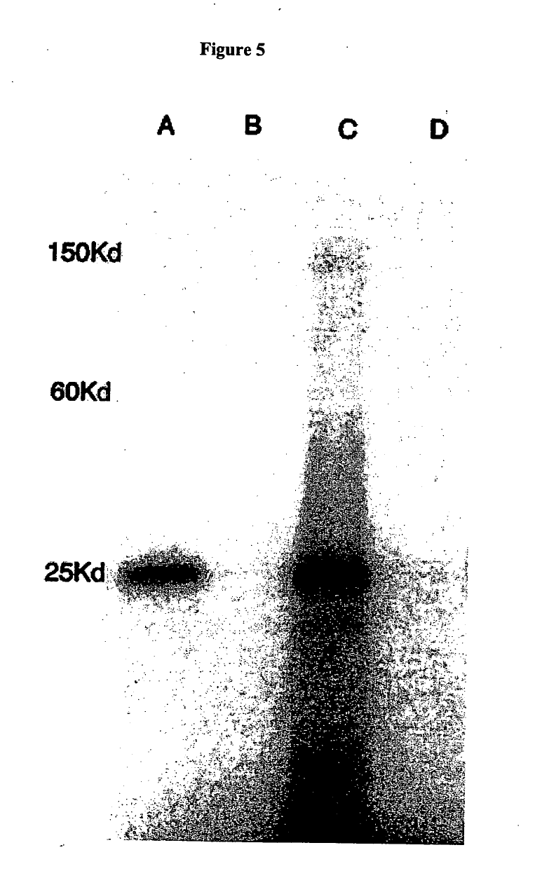

As reactive ligands, a small set of chelates bearing electrophilic para substituents (FIG. 3) that could react with cysteine were prepared. To be most useful, the electrophilic ligands should be stable in biological media so that they not react prematurely with common biological nucleophiles such as the cysteine on glutathione or in albumin (Geigy Scientific Tables Vol 3, C. Lentner, ed., Ciba-Geigy Ltd., Basel, Switz (1984)). The whole-body clearance of the radiolabeled chelates from mice was studied (FIG. 4 and Chmura, A. J., et al., J. Controlled Release 78:249-258 (2002)) to identify the electrophilic chelates that are unreactive in plasma in vitro, and which also quantitatively clear from live animals. The acryl compound AABE showed the required clearance properties. No less important, AABE-¹¹¹In reacted with the S95C mutant of CHA255 to permanently tag the light chain (FIG. 5).

To investigate whether either the S95C or the N96C Fab would bind irreversibly to its target, ¹¹¹In-labeled chelates bearing electrophilic groups were incubated at physiological pH and temperature with raw tissue culture medium from the cells expressing these recombinant proteins. The culture medium contained 10% fetal calf serum, representative of typical biological media. The specific covalent attachment of an ¹¹¹In-chelate to a mutant Fab—under conditions where it does not covalently attach to other molecules such as albumin present in the culture medium, or to the native Fab (it binds reversibly to the native Fab)—illustrates the utility of this approach.

The samples were analyzed by denaturing gel electrophoresis (SDS-PAGE). Separation under reducing and denaturing conditions on SDS-PAGE separates the light chain from the heavy chain of each Fab, functionally destroying the antibody-binding pocket. Chelates bound to the Fab but not covalently linked will dissociate because the antibody-binding pocket is no longer folded. Unbound chelates do not migrate with the antibody chains. However, a chelate that not only bound to a Fab but also covalently linked will be attached to the Fab light chain and migrate with it on SDS-PAGE. We saw this result with the Fab S95C; nucleophilic Fab S95C reacted equally well with the very electrophilic ¹¹¹In-chloroacetamidobenzyl-EDTA chelate (FIG. 5 lane A) and with the weakly electrophilic ¹¹¹In-AABE chelate (FIG. 5 lane C), but not with electrophilic ¹¹¹In-(S)-1-p-chloropropionamidobenzyl-EDTA or non-electrophilic ¹¹¹In-ABE (FIG. 5 lanes B and D). We did not observe any covalent cross-linking between the native Fab and any of the electrophilic chelates. Nor did we observe cross-links when the nucleophilic Fab N96C was studied: apparently the orientation of the Cys in position 96 is inferior to position 95 for reaction with these ligands.

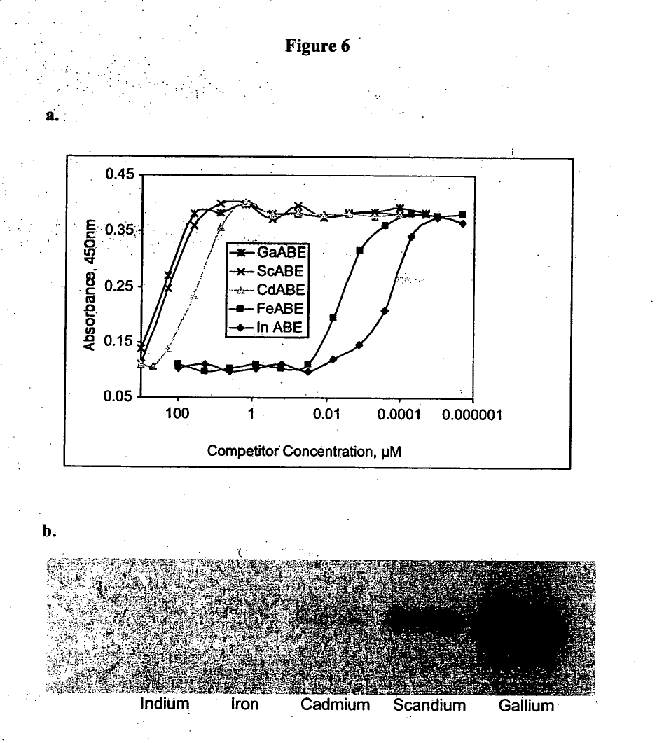

The parental mAb CHA255 has exquisite selectivity for binding to benzyl-EDTA chelates bearing different chelated metals (In, Fe, Cd, Sc, Ga) (see, e.g., Dayton T. et al., Nature 316, 265-268 (1985)). To be assured that mutation of residue 95 in the Fab binding site had no deleterious effects on the selectivity of the Fab for benzyl-EDTA complexes we analyzed the metal selectivity of the S95C mutant Fab by competitive ELISA (FIG. 6a). The concentration of each competitor that is effective at blocking the attachment of S95C Fab to the plate is related to the competitor's affinity for binding to the antibody and is practically identical to the native antibody under the same conditions. The present ELISA results show that either wild-type or S95C mutant antibody favors binding to indium chelates relative to others by free energy differences (AAG, kJ/mol) of approximately 7.6 (Fe^{III}), 30 (Cd^{II}), 34 (Sc^{III}), or 35 (Ga^{III}).

To further confirm that S95C Fab retained the ligand-binding specificity, we performed a competition assay to see whether nonradioactive ABE-indium, -iron, -cadmium, -scandium, or -gallium could block the covalent attachment of ¹¹¹In-AABE. As shown in FIG. 6b, excess ABE-indium effectively blocks the reaction, whereas the other metal chelates are less effective, in order of their relative affinities.

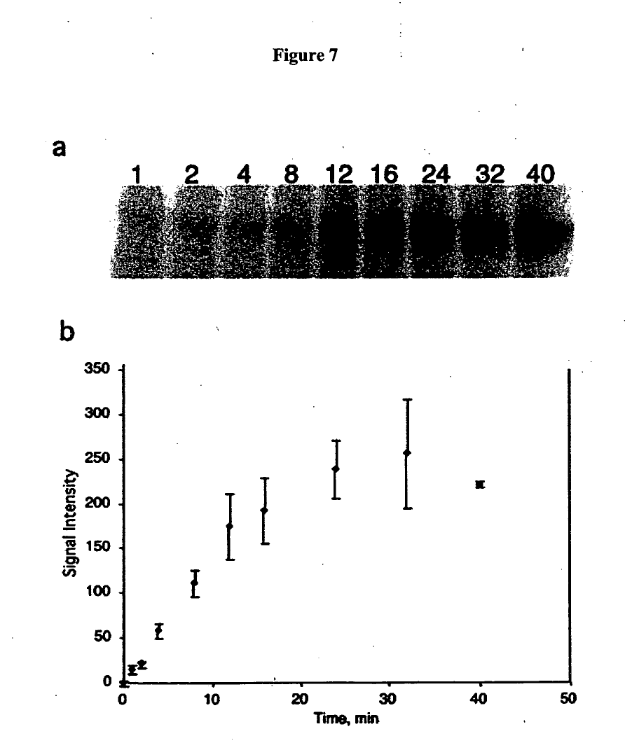

We explored the rate of covalent attachment of the AABE-¹¹¹In chelate to the S95C mutant, by monitoring the extent of reaction as a function of time. The covalent attachment was 50% complete in approximately 10 min at 22° C. (FIG. 7). This is shorter than the typical 50- to 100-min bound half-lives of complexes between the native CHA255 antibody and nonelectrophilic ligands of similar structure under similar conditions (Meyer, Damon L. et al., Bioconjugate Chem. (1990), 1(4), 278-84), suggesting that when AABE-indium binds to S95C, it forms a covalent bond with high efficiency.

### Example 2

### High Affinity of 2D12.5 Antibody for Rare-Earth DOTA Complexes

This example illustrates the broad specificity and high affinity of the 2D12.5 antibody for rare earth-DOTA complexes that make the antibody particularly interesting for applications that take advantage of the unique characteristics of lanthanides.

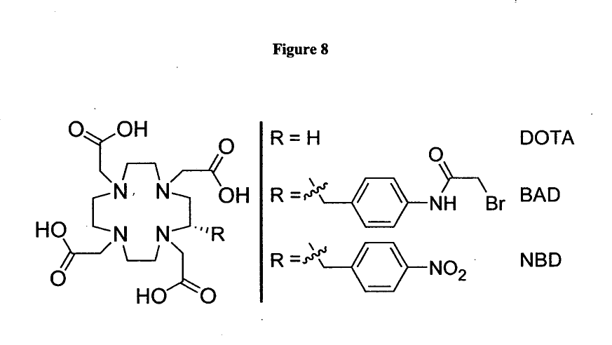

The rare earths are rich in probe properties, such as the paramagnetism of Gd, the luminescence of Tb and Eu, and the nuclear properties of Lu and the group IIIB element Y. The chelating ligand DOTA binds transition metals and rare earths with extreme stability under physiological conditions, leading to its use in vivo. Therefore, the monoclonal antibody 2D12.5 (David A Goodwin et al., Journal of Nuclear Medicine, 33, 2006-2013 (1992)) developed against the DOTA analogue Y-BAD conjugated to the immunogenic protein KLH through a 2-iminothiolane linker and selected to bind specifically to Y-NBD (FIG. 8), was examined to determine the scope of its activity.

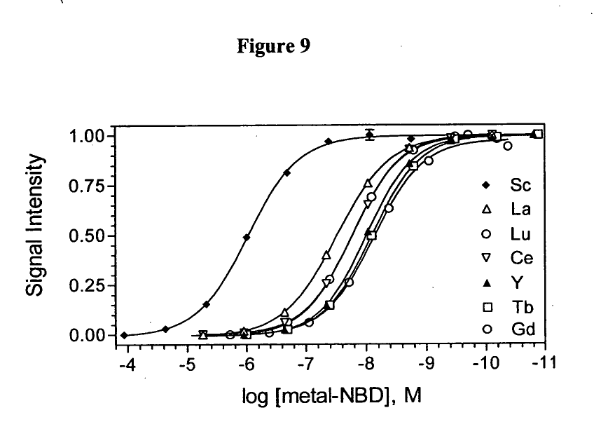

A competitive immunoassay to measure the binding constants of various metal-(S)NBD complexes relative to the original Y³⁺ complex (FIG. 9) was developed to assess the metal selectivity of antibody 2D12.5. Briefly, 2D12.5 was incubated at 37° C. in the presence of immobilized HSA-21T-Y-(S)BAD and a soluble metal-(S)NBD competitor (Y-(S)BAD was linked to human serum albumin via 2-iminothiolane). The metal-(S)NBD concentration was varied from μM to pM in order to determine the relative binding affinity of 2D12.5 for each metal chelate in comparison to Y-(S)NBD. We found that 2D12.5 binds not only Y-(S)NBD but also (S)NBD complexes of all the lanthanides. Surprisingly, some metal chelates such as Gd-(S)NBD bind more tightly than the original Y³⁺ complex; overall, the dissociation constants fall within a factor of 3 above or below the K_{D}=10 nM value for Y-(S)NBD (The overall ΔG° of binding for Y-(S)NBD is −46 kJ/mol). Other antibodies that bind metal chelates do so with a strong preference for one or possibly two metals (Love, R. et al., Biochemistry 32, 10950-10959 (1993), and Khosraviani, M., et al., (2000) Bioconjugate Chem., 11, 267-277). For example, the (S)NBD chelate of group IIIB ion Sc(III) binds to the antibody with a much lower affinity (<1%) than the strongest binding rare earth complexes, perhaps because Sc(III) has a much smaller ionic radius (Meehan, P. R., et al., Coord. Chem. Rev., 181, 121145, and Shannon, R. D. (1976) Acta Crystallogr., Sect. A: Found. Crystallogr., A32, 751-767).

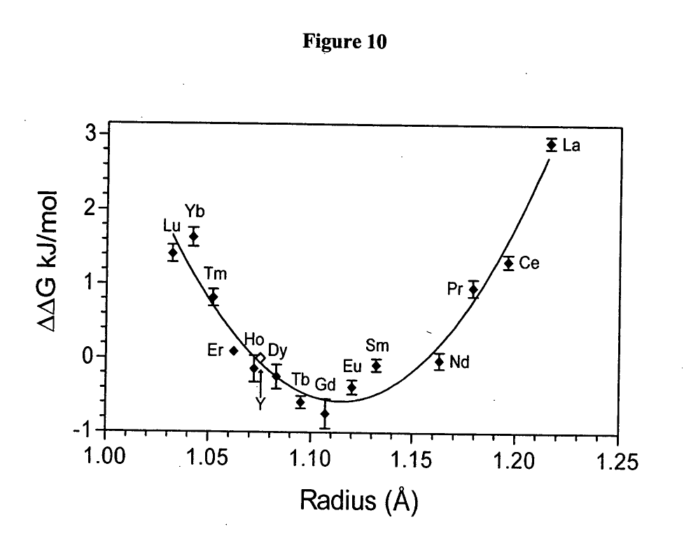

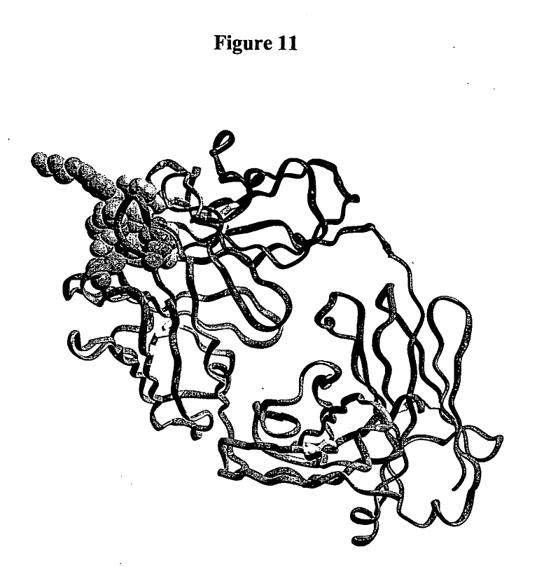

The relative binding affinities determined for each rare earth (S)NBD complex relative to Y-(S)NBD are plotted as G values in FIG. 10. Out of 15 ions tested, six rare earth complexes had G values more favorable for binding than the original Y³⁺ complex. The radii of the nonacoordinate trivalent lanthanide ions vary in small increments across the series from 1.21 Å (La³⁺) to 1.03 Å (Lu³⁺).

Our results show that when the shape of the (S)NBD complex is perturbed by either increasing or decreasing the radius of the lanthanide ion, the stability of the protein-ligand complex changes in a regular fashion. The effect of the change in ionic radius on the standard G of binding should be described approximately by an equation of the form:

$$\frac{ⅆ Δ G}{ⅆ r} = k \vert r - {r}_{0} \vert ,$$

which integrates to

$$Δ Δ G = \frac{1}{2} {k ( r - {r}_{0} )}^{2} .$$

The behavior of G as a function of ionic radius fits a parabola, as might be expected for a system that behaves in a thermodynamically elastic way, obeying Hooke's law over a small range of perturbations (Comeillie, T. M., et al., (2003) Journal of the American Chemical Society 125, 3436-3437).

Thus, the broad specificity and high affinity of this antibody for rare earth-DOTA complexes make it particularly interesting for applications that take advantage of the unique characteristics of lanthanides. For example, it could be used as a docking station for a whole set of probe molecules of particular interest for medical imaging and therapy.

### Example 3

### Structural Determination of Y-(S)—HETD-2D12.5 Fab Complexes

The following example illustrates the crystal structure determination of Y-(S)-HETD-2D12.5 Fab Complex.

Sequencing of variable domains of 2D12.5. Poly-adenylated mRNA was purified from 2D12.5 hybridoma cells by standard techniques. cDNA was obtained using Novagen's Mouse Ig-Primer kit, which incorporates degenerate 3′ constant domain primers specific to mouse IgG genes. Double stranded DNA was obtained from cDNA using degenerate 5′and 3′primers provided in the Mouse Ig-Primer kit. The heavy and light chain variable genes, each with an unpaired 3′terminal A, were cloned separately into a pT7Blue T-vector and sequenced. The constant domain sequence of the light chain was later obtained from poly-A mRNA using degenerate primers, while limited attempts to obtain the sequence of the CH1 domain were unsuccessful. Analysis of the Kabat database led to the selection of a consensus sequence for the CH1 domain that was used to solve the crystal structure; the electron density supports the Kabat derived consensus sequence.

Antibody Deglycosylation. Observation of 2D12.5 Fab heterogeneity by SDS-PAGE, and identification of an Nlinked glycosylation sequence at position 85 (Kabat numbering) of the heavy chain led us to deglycosylate the antibody with Endo F2. Approximately 100 mg of antibody 2D12.5 purified from hybridoma cell culture (˜8.4 mg/mL) was dialyzed into 50 mM NaOAc, pH 4.5. Endo F2 was added (1.6 μU per ∝g of total protein), and the solution was placed in a 10,000 NMWL dialysis cassette and allowed to incubate for several days at 37° C. A large amount of protein was treated with a relatively small amount of Endo F2 (recommended 50 μU per ∝g protein); therefore, the outer 50 mM NaOAc, pH 4.5 buffer was exchanged daily to minimize enzyme inhibition by cleaved glycans. The enzymatic reaction was monitored by SDS PAGE.

2D12.5 Fab Preparation. Deglycosylated antibody 2D12.5 (approximately 100 mg) was dialyzed into a neutral buffer (20 mM sodium phosphate, 10 mM EDTA, pH 7). The protein solution was diluted by half into the same buffer containing cysteine (20 mM) immediately prior to the addition of 10 mL papain gel (immobilized on cross-linked, 6% beaded agarose) pre-equilibrated in the same buffer. The mixture was agitated for 16 h at 37° C. and digestion progress was monitored using a Superdex 200 HR 10/30 gel filtration column equilibrated in CAPS buffer. The immobilized papain was removed by centrifugation and filtration, and the resulting solution was concentrated by centrifugation using an Ultrafree-10 protein concentrator (Millipore). Fab and contaminating Fc fragments of the same relative size were separated from undigested antibody and small proteolytic fragments by gel filtration. CAPS buffer (pH 10) was used because Fab fragments were found to have low solubility at neutral pH and concentrations greater than 1 mg/mL. Two methods were evaluated to separate Fab from comparably sized Fc fragments. Protein A, which is known to have a low affinity for mouse IgG1 Fc (Akerstrom, B. et al., J. Immunol., 135, 2589-259296), did not sufficiently remove the contaminating fragments. The Fab fragments were successfully purified by an alternate strategy using immobilized Protein G, which has a weak affinity for the CH1 (Fab) domain of mouse IgG1 antibodies (Derrick, J. P et al., (1992) Nature, 359, 752-7597). The purified Fab was dialyzed extensively into CAPS buffer and concentrated to 9.6 mg/mL. A noncompetitive ELISA was used to confirm the ligand binding activity of the purified Fab solution relative to undigested antibody.

Protein-Ligand Crystallization. Yttrium-(S)HETD, a hapten having a sidechain similar to the original antigen, was used in the crystallization. The protein-ligand complex was prepared by incubating 1.8 equivalents of Y-(S)HETD with the purified Fab (9.7 mg/mL) for several minutes. Final concentrations of protein and ligand in a typical sample used for crystallization were 190 μM and 340 μM, respectively. This solution was screened for crystallization conditions by hanging-drop vapor diffusion. A typical drop contained 4 μL of a 1:1 mixture of protein-ligand solution and crystallization solution (100 mM HEPES pH 7.5, 18-20% PEG 8000). Crystals of the space group P212121 appeared within 2 days at 290 K as thin plates with the approximate dimensions 0.75 mm×0.25 mm×0.05 mm. Crystals were transferred into a cryo-protectant solution (100 mM HEPES, 22% PEG 8000, 75 mM NaCl and 20% ethylene glycol) and allowed to equilibrate overnight before cryocooling under a N2 gas stream at 100 K. After determining the conditions for crystallizing the Y-(S)HETD bound 2D12.5 Fab, the Gd³⁺ complex of (S)NBD (an analogous chelate with a shorter sidechain, FIG. 8) was incubated with the purified 2D12.5 Fab and crystallized using the same methods described for the Y-(S)HETD hapten. Protein crystals did not form in the absence of either metal complex. Data collection, phase determination, model building and refinement of Y-(S)HETD (or Gd-(S)NBD) bound to 2D 12.5 Fab were carried out as described (Comeillie, T. M., et al., (2003) Journal of the American Chemical Society 125, 15039-15048).

Observed Isomers. When the DOTA macrocycle wraps around a metal ion, it assumes a helical twist. A bulky substituent on a backbone carbon of the macrocycle can determine the handedness of the helix. Fits to the electron density indicate that only the expected 7(δδδδ) enantiomers of Y-(S)HETD and Gd-(S)NBD are observed in the ligand-bound crystal structures of mAb 2D 12.5.

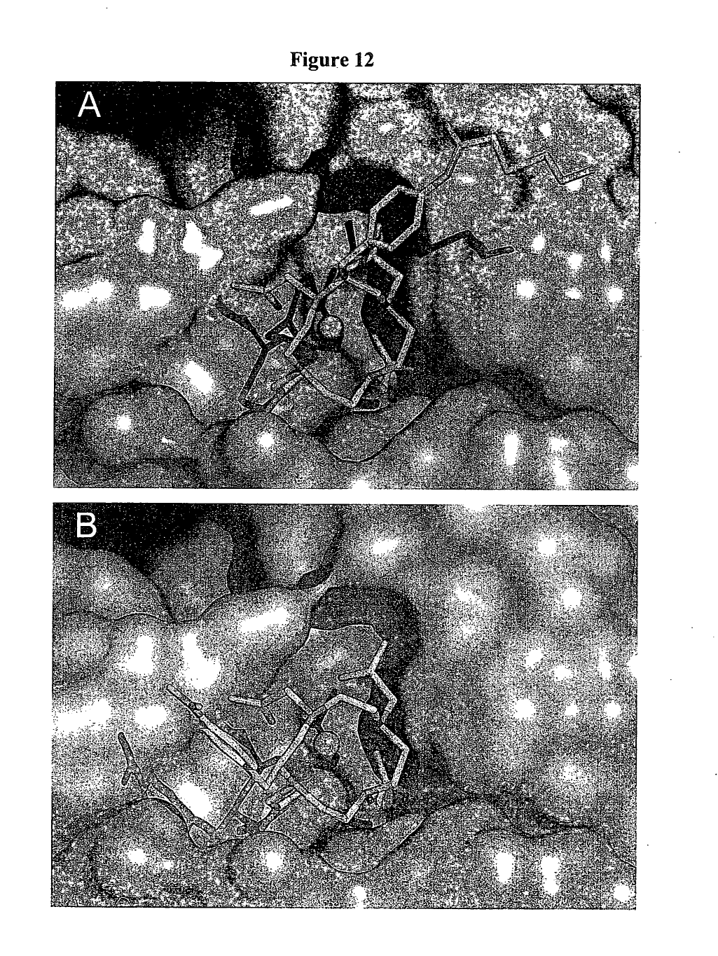

The (S)HETD and (S)NBD sidechains do not appear to interact with the protein in either structure (FIG. 12). Y-DOTA and Gd-DOTA exhibit C4 symmetry; a repeated 90° rotation of the molecule around the central axis yields the same molecule. Incorporation of a (S)HETD or (S)NBD sidechain destroys the C4 symmetry.

For each complex, the orientation that is best resolved in the electron density maps is represented in the final structures in FIG. 12. The metal-DOTA moiety lies at an angle in the binding cavity. The other possible orientations are prevented because of potential steric clashes between the (S)HETD and (S)NBD side chains with the protein. A 90° rotation of the DOTA moiety represents the difference between the two possible (S)HETD and (S)NBD sidechain orientations. The other two orientations would place the sidechain too close to aromatic sidechain residues Trp52 (CDR2(H)), Tyr32(CDR1(L)) or Trp91 (CDR3(L)). An unsubstituted metal-DOTA complex would not have the sidechain interference presented by (S)HETD and (S)NBD, and so could bind in any of four equivalent orientations.

Binding Interactions. Analysis of the binding cavity indicates that there are no significant perturbations of the protein backbone or protein sidechains between the structural models of 2D12.5 bound to Y-(S)HETD and Gd-(S)NBD. Any movement of the protein between the structures is within the RMSD values for the protein structure. The specific binding interaction between antibody 2D12.5 and its ligands has an interesting flexibility that allows the substitution of any lanthanide ion into the DOTA moiety. The different metal-DOTA complexes show quantitative, but not qualitative, differences in binding affinity 94 The structure provides several insights into this. First, there is no direct interaction between the metal and the protein. The DOTA moiety fills eight of nine available coordination sites for either Y³⁺ or Gd³⁺. An inner sphere water molecule fills the final coordination site of the metal and is observed in both the Y-(S)HETD and Gd-(S)NBD structures.

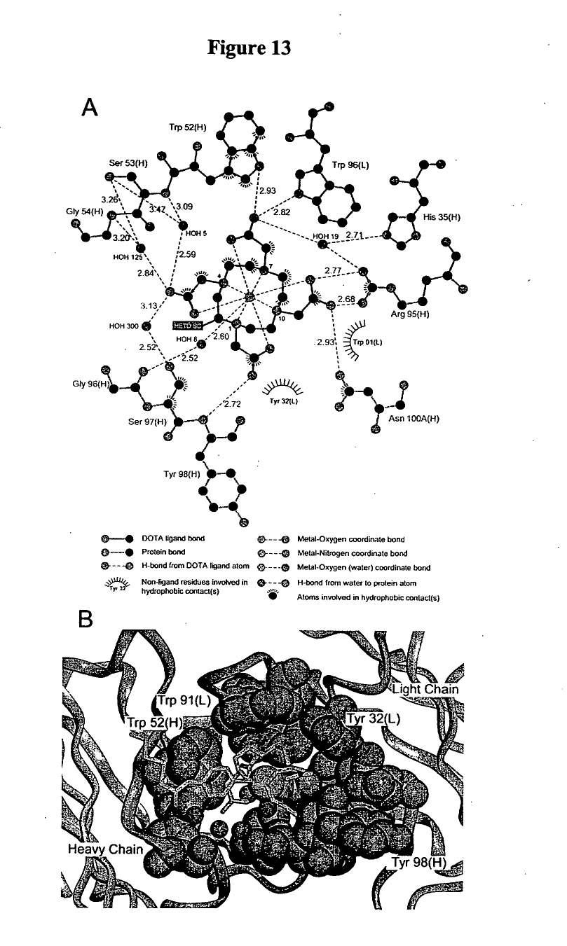

The binding interactions between the ligand and the antibody include a bidentate salt bridge, five direct H-bonds, four to five water-mediated H-bonds and numerous hydrophobic contacts (FIG. 13). The DOTA moiety forms an amphipathic cylinder with the charged carboxylate groups toward the face of the chelate near the metal ion, while nonpolar methylene groups from the macrocycle and the carboxymethyl groups occupy the rear and sides of the molecule. The net charge of the metal-DOTA complex is negative (−1), and this charge is centered near the face of the coordinating carboxylates, where most of the polar interactions occur. The single most obvious attachment is the bidentate salt bridge between a DOTA carboxylate and an arginine side chain, Arg95(H), at the bottom of the binding cavity (right side of FIG. 13).

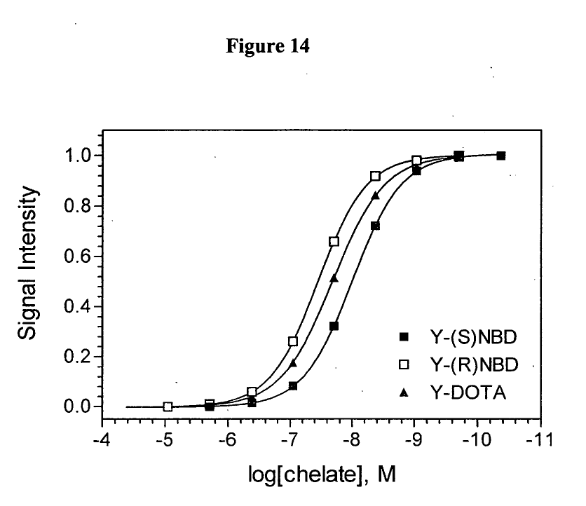

Enantiomeric selectivity. The symmetrical nature of the metal-DOTA moiety of Y-(S)HETD and Gd-(S)NBD led us to examine the enantioselectivity of 2D 12.5. Metal chelates containing polydentate ligands such as DOTA form enantiomeric complexes having opposite helicities. Other monoclonal antibodies developed to bind chiral metal complexes have demonstrated high selectivity for only one of the enantiomers (Dayton T., et al., Nature 316, 265-268 (1985); Bosslet, K. et al., (1991) Br. J. Cancer, 63, 681-686, and Blake, D. A., et al., (1996) J. Biol. Chem., 271, 2767727685). But we discovered that antibody 2D12.5 binds Y-(R)NBD with a ΔΔG=3.3 kJ/mol, relative to the Y-(S)NBD complex (FIG. 14). Interestingly, the affinity of antibody 2D12.5 for Y-(R)NBD is similar to its affinity for La-(S)NBD (ΔΔG=2.9 kJ/mol). For reference, G⁰=−45.7 kJ/mol for Y-(S)NBD binding to 2D12.5.

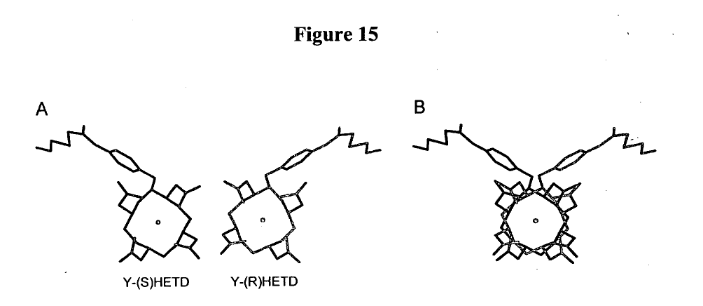

For the protein to bind both enantiomers of Y-NBD, the chiral nature of the ligand must not significantly alter the relative position of heteroatoms or hydrophobic groups necessary for high affinity binding. In fact, the DOTA moiety of 3-dimensional models of Y-(S)NBD and Y-(R)NBD having opposite DOTA helicities can be superimposed without much altering the relative location of carboxylate oxygens important for binding to the antibody (FIG. 15). This is a result of the approximately cylindrical shape and symmetry of the metal-complexed DOTA moiety. Consistent with these results, the binding affinity of Y-DOTA without a side chain is measured to be lower than Y-(S)NBD but higher than Y-(R)NBD. Y-DOTA molecules are present in solution with both helicities in equal concentration.

### Example 4

### Engineering Single Cysteine Residues Near the Binding Site of the 2D12.5 Antibody

The following example illustrates how 2D12.5 was made even more useful for biological applications by engineering single cysteine residues near the protein's binding site.

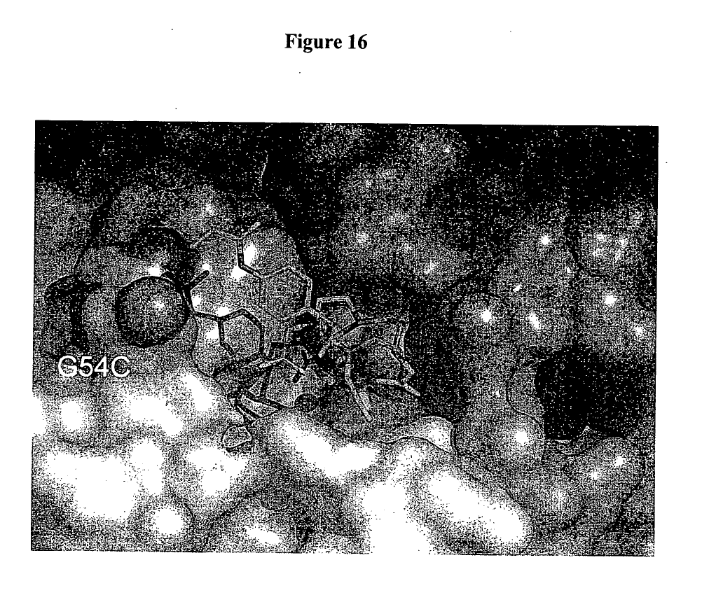

Three consecutive glycine residues that do not contact the ligand are located in a loop of the heavy chain, near the ligand p-substituent. Three mutant genes were constructed, and expressed in S2 insect cells. The G54C mutant (FIG. 16) was selected for more extensive study because it had the best expression level and activity. The G54C mutant binds reversible ligands such as Y-NBD with affinities comparable to the native protein.

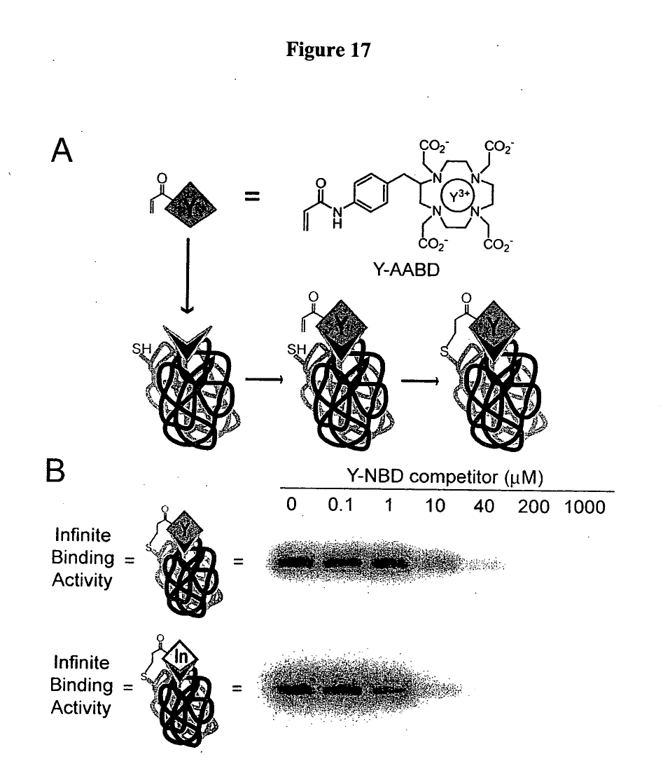

Y-AABD (FIG. 17) was synthesized and used as a candidate irreversible ligand. This yttrium complex bound reversibly to parental 2D12.5 with K_{D}=4·10⁻⁹ M, comparable to the reversible ligand Y-NBD. ⁹⁰Y-AABD was prepared and tested it for permanent binding to the G54C mutant. The results in FIG. 17B show that at 37° C., pH 7.5, ⁹⁰Y-AABD permanently and specifically attaches to the G54C mutant.

In addition, a new aspect of infinite binding was discovered: the reversible In-NBD chelate binds only weakly, but the acryl ligand ¹¹¹In-AABD binds permanently to the G54C mutant. FIG. 17B shows that ¹¹¹In-AABD binds specifically to G54C, with a yield similar to the stronger ligand ⁹⁰Y-AABD. An important practical implication is that the G54C mutant of 2D12.5 may be used for applications that include not only radiotherapy with ⁹⁰Y, but also imaging with ¹¹¹In. The range of metals with useful probe properties thus extends beyond the rare earths (K_{D}˜10⁻⁸ M for reversible binders) to scandium and indium, whose complexes do not have useful affinities as reversible binders (K_{D}˜10⁻⁶ M), and perhaps beyond.

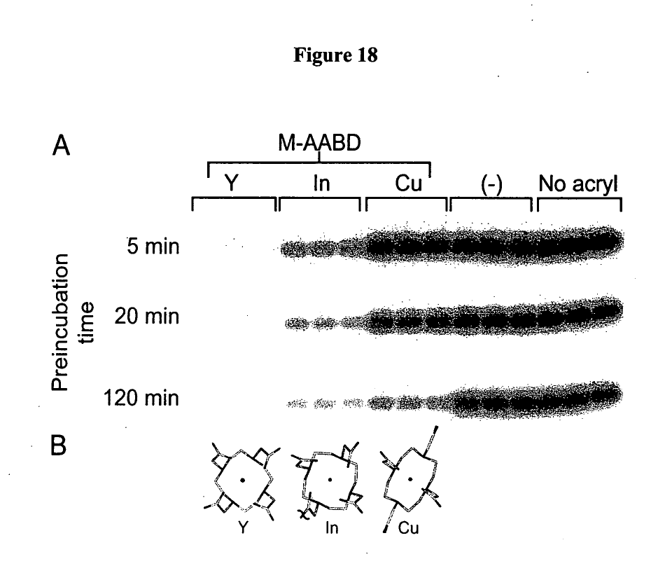

We were curious to see if an even weaker binder such as a copper chelate (K_{D}˜10⁻⁴ M for Cu-NBD) would bind irreversibly under ordinary conditions. We tested this by comparing the permanent binding of Y³⁺-, In³⁺-, and Cu²⁺-AABD, using competitive ⁹⁰Y-AABD attachment as an indicator (FIG. 18). Strongly binding Y-AABD does not allow a significant amount of 90Y-AABD to attach to the G54C: ⁹⁰Y-AABD is blocked from >90% of the sites after preincubation with Y-AABD for 5 min or more. Weaker binding In-AABD blocks >50% of the sites after 5 min preincubation, and even more at later times. Very weakly binding Cu-AABD initially occupies only about 10% of the G54C sites under these conditions, but it has permanently attached to G54C in good yield after 2 hr, blocking>70% of the sites.

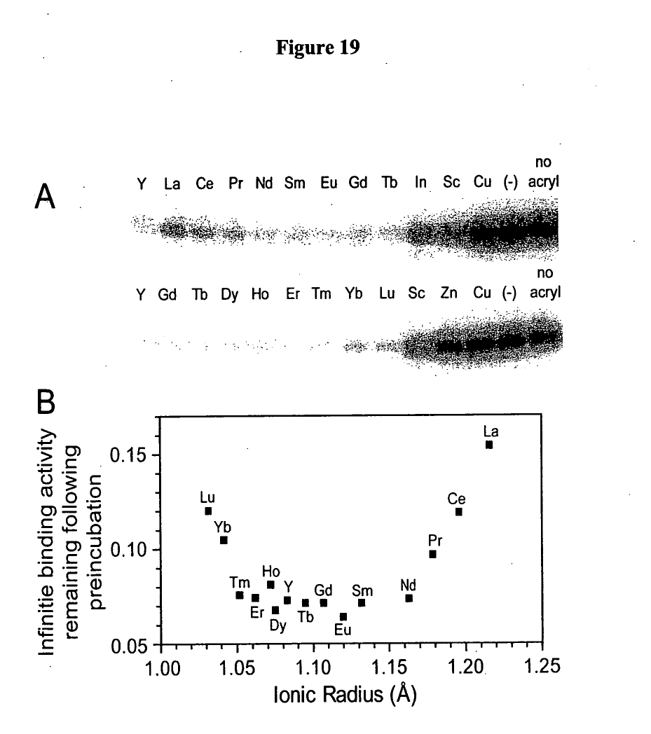

In accord with their K_{D} values, evidence from crystal structures shows significant differences in preferred coordination geometry between the DOTA complexes of yttrium, indium and copper (FIG. 18B) (Liu, S., et al., Inorg. Chem., 42, 8831-8837; Chang, C. A., et al., (1993) Inorg. Chem., 32, 3501-3508; Parker, D. et al., (1994) J. Chem. Soc., Dalton Trans., 689-693; Riesen, A., et al., (1986) Helv. Chim. Acta, 69, 2067-2073; and Cosentino, U., et al., (2002) J. Am. Chem. Soc., 124, 49014909). Control experiments with two acrylamido compounds having no measurable affinity for 2D12.5 show no permanent attachment to G54C, indicating that ligand binding is required for the covalent bond-forming reaction to occur. DOTA complexes of all the trivalent rare earth ions bind to 2D12.5 with similar affinities. Upon careful examination, the free energies of binding the rare earth-DOTA complexes to the antibody show a parabolic dependence on the ionic radius of the metal (FIG. 10), with Gd at the bottom and La and Lu at the extremes. FIG. 19 shows that the rare earth-AABD complexes bind permanently to G54C in yields that correlate with their relative affinities. All the 90% while the stronger ones have yields of 90-95%.

These results demonstrate that an infinite binding system can exhibit selective and permanent attachment with a surprising range of structurally related ligands, albeit at slower rates as affinities decrease. Separately engineering the reactivities of both ligand and receptor provides a direct route to the capture of a set of similar molecules, with unsurpassed affinity.

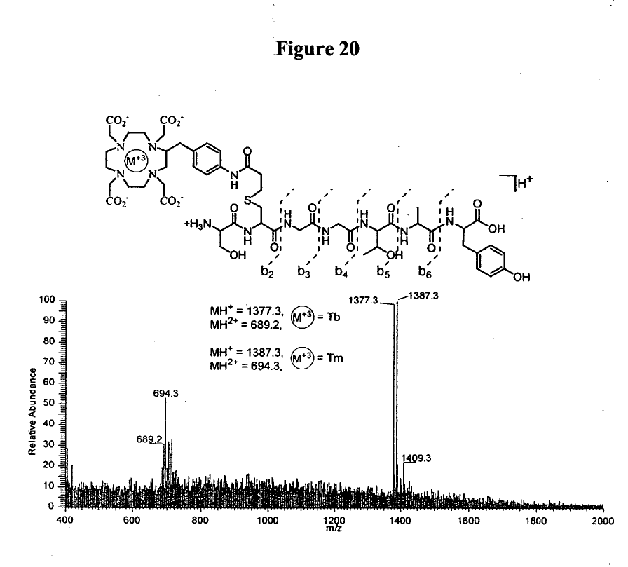

To be certain that the G54C mutant was being specifically labeled at the engineered cysteine site, further characterized the permanent bond formation between the G54C mutant and rare earth complexes of AABD. We used the new methodology of element-coded affinity tags (ECAT) to simplify identification of the labeled site (Whetstone, P. A. et al., Bioconjugate Chem 2004, 15, 3-6). Briefly, we incubated equal aliquots of G54C Fab with either Tb- or Tm-AABD, removed unlabeled AABD complexes by gel filtration and mixed the two protein samples. The combined sample was then digested with chymotrypsin, as the trypsin fragment would have been too large to identify conveniently by mass spectrometry. Any peptides incorporating a metal-AABD label were affinity separated from unlabeled peptides using an immobilized 2D12.5 affinity column. Since the labels co-elute on reverse-phase liquid chromatography, they provide a doublet signature in the mass spectrum. The masses of any peptides labeled with Tb-AABD or Tm-AABD differ by 10 mass units, a mass differential not commonly found in proteins. A search of the LC data identified only a single peptide that had a mass differential of 10 mass units. MS/MS confirmed that the sequence of the peptide was SCGGTAY, and the cysteine was labeled with either Tb-AABD or Tm-AABD (FIG. 20). This peptide incorporates the purposefully placed G54C mutation.

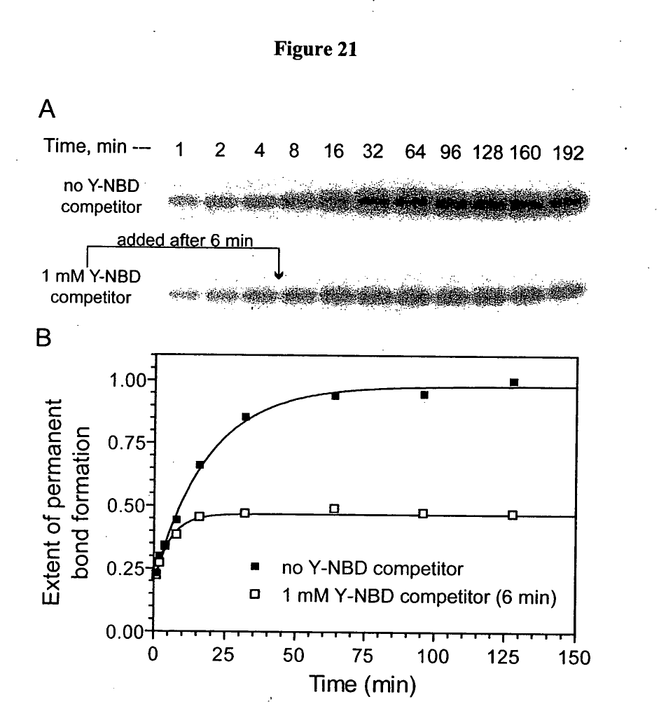

The LC/MS labeling experiments indicate that the covalent link between the G54C Fab mutant and metal-AABD complexes is specific to residue Cys-54 of the heavy chain, as expected. Further evidence about permanent bond formation was collected through kinetics experiments, one of which incorporated the reversibly binding competitor Y-NBD (FIG. 21).

The question to be addressed was the probability that Y-AABD reacts with G54C the first time it forms a complex, instead of dissociating and rebinding. In the first experiment ⁹⁰Y-labeled Y-AABD (10 μM) was added to G54C Fab (1 μM) at 37° C., pH 7.5. Samples were withdrawn at various times and were immediately added to SAB, boiled and reduced under denaturing conditions to halt any further permanent bond formation. Samples were analyzed by SDS-PAGE, and the extent of permanent bond formation was determined by measuring the relative radioactivity of bands. The time to 50% permanent bond formation was t_{1/2} approximately 13 min.

In a second experiment, performed in parallel, conditions were identical to the first except that the reversible competitor Y-NBD was added in vast excess (1 mM) relative to Y-AABD after 6 min, allowing labeled Y-AABD complexes to form bonds or dissociate, but competitively blocking any re-binding of Y-AABD to G54C.

There are two possible binding modes for the Y-AABD ligand to select as it binds to antibody 2D12.5. Thus, Y-AABD will distribute between the two binding modes i, and only one of these modes is favorably positioned to react with the G54C mutant. Because it is in large excess, the Y-NBD blocks any Y-AABD that may have initially bound in the unproductive orientation from rebinding to form the permanent covalent bond with the G54C Fab.

The experiment containing no competitor, on the other hand, allows any Y-AABD molecules initially bound in the mode that is disfavored for permanent bond formation to redistribute between the two orientations until all reactive sites are filled and permanent binding reaches a maximum. Note that the overall t_{1/2} of approximately 13 min observed in the first experiment includes the time required for reorientation of the Y-AABD ligand in the binding pocket.

A very short t_{1/2} for permanent binding was observed in the second experiment (of approximately 4 min). We expect that the 4 min half-time measured in the second experiment specifically describes the half-time for permanent bond formation for Y-AABD ligands bound in the productive orientation. Thus, there is a nearly equal distribution of the two binding modes, implying that about half the Y-AABD molecules react upon initial complex formation.

These experiments demonstrate that the Y-AABD ligands bind sufficiently for most applications. However, other ligands can be designed that might give different yields upon first association—perhaps lower if there turns out to be a binding site barrier for the ligand, or higher to maximize the efficiency of probe capture.

### Example 5

### Engineering of Lym-1 Single Chain Single-Cysteine Mutants for Irreversible Binding to HLA-DR

The following example illustrates the preparation of a library of single-Cys mutants based on the Lym-1 single-chain antibody fragment sL1. The mutants of this library can be used with site-selective cross-linking reagents to bind the antibody to a tumor antigen.

We have chosen to mutate the hypervariable sequence positions of the antibody (the six complementarity determining regions (CDRs) identified by Kabat and co-workers http://www.kabatdatabase.com/) because they are least likely to be critical for the structural integrity of the antibody and most likely to be located near the antigen. The 6 CDR regions of Lym-1 contain a total of 57 residues.

TABLE 3
Matrix approach used for determining optimal
mutant/linker combinations.

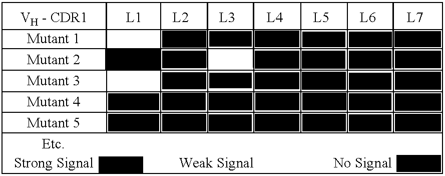

*No specific data is represented in this table but it is used to illustrate how a matrix with a reactivity coordinate (L1-L7) and a proximity coordinate (mutant 1-5), will allow for optimal mutant/linker pairs to be identified.

A rational approach was taken in which it was hypothesized that at least one naturally occurring reactive group would be present within the general vicinity of the binding epitope, based on average distribution frequencies of appropriate amino acid side chains. Thus, a scFv engineered with a reactive linker in the vicinity of the binding pocket would orient a reactive linker and a naturally available side chain appropriately for cross-linking. A matrix approach was adopted to identify candidate mutant/linker pairs showing high covalent reactivity with the natural antigen. Table 3 is a representative example of this approach where linker reactivity (L1-L7) was investigated with differing mutants. The mutant library, which comprises up to ≈50-60 mutants, effectively moves the linker molecule around the periphery of the binding site, probing for surface reactive groups in close proximity.

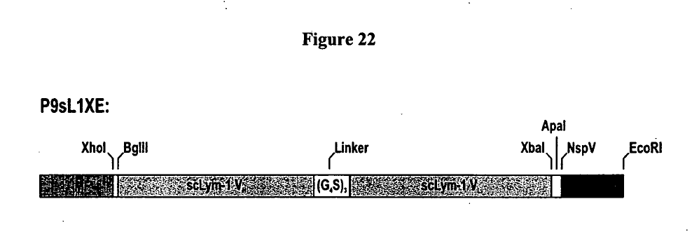

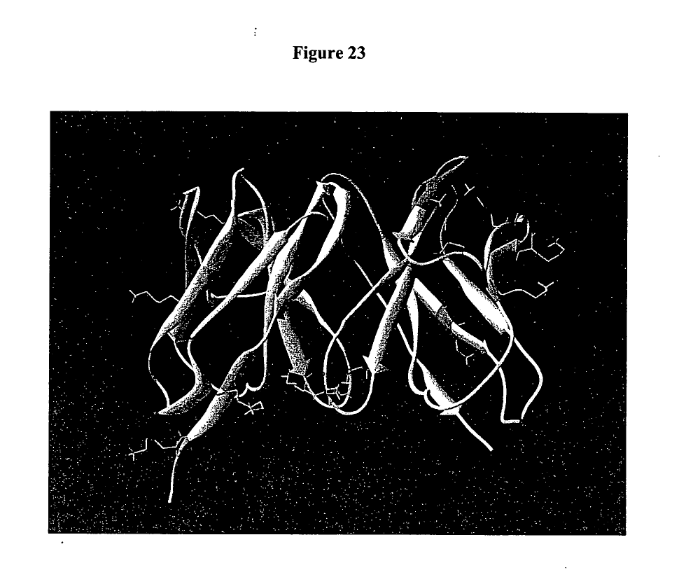

A library of single cysteine mutations was constructed based on the Lym-1 single-chain expression cassette presented in FIG. 22. These mutations were located in areas that were predictably close to the binding site by mutating amino acids in the complementarity determining regions (CDRs) of the antibody. Because the CDRs are generally predictable for all antibodies without the need for a crystal structure, this particular methodology for engineering irreversible binding is broad in scope and applicability. In addition, because the library of linkers targets a common reactive group in native proteins, there is no need for specific structure knowledge of the target, further expanding the generality of this methodology.

The method of Ito et al. ((1991) Gene, 102, 67-70) was used on the DNA template in FIG. 22 to replace single CDR residues with cysteine to facilitate chemical activation with a selection of reagents (see below). Of the 57 CDR residues of Lym-1 that made up our initial library, we have cloned 43 (75%). All sequences have been verified and are generally mutation-free apart from the desired single-Cys substitution. At this time, 37 of these have been transformed into P. pastoris and expressed (confirmed by western blot of the culture media). Table I gives an overview of the current status.

TABLE 1
sL1 Cloning and Expression^{a}

| | | | |
|---|---|---|---|
| | | Transformed | |
| Kabat | Cloned | P. pastoris | Expressed |

| | | | | |
|---|---|---|---|---|
| VH - CDR1 (SEQ ID NO:10) | SYGVH | SYGVH | SYGVH | SYGVH |
| VH - CDR2 (SEQ ID NO:11) | VIWSDGSTTYNSALKS | VxWSxxxxTYxSxxS | VxWSxxxxTxxSxxxS | VxWSxxxxTxxSxxxS |
| VH - CDR3 (SEQ ID NO:12) | HYGSTLAFAS | HYGSTxxxxS | xYGSTxxxxS | xYGSTxxxxS |
| VL - CDR1 (SEQ ID NO:15) | RASVNIYSYLA | RASVNIYSYLA | RASVNIYSYLA | RASVNIYSYLA |
| VL - CDR2 (SEQ ID NO:16) | NAKILAE | xAKILAE | xAKILxx | xAKILxx |
| VL - CDR3 (SEQ ID NO:17) | QHHYGTFT | QHHYGTFT | xxHYGTFT | xxHYGTFT |

^{a}Lower-case “x” denotes a residue not currently investigated or isolated in indicated experimental stage.

The nucleophilic lysine Σ-amino groups in the sL1 molecule are distributed such that most of the CDR residues are several Å away, so a single-Cys mutant tagged with a short cross-linker is not likely to inactivate itself by forming an intramolecular cross-link.

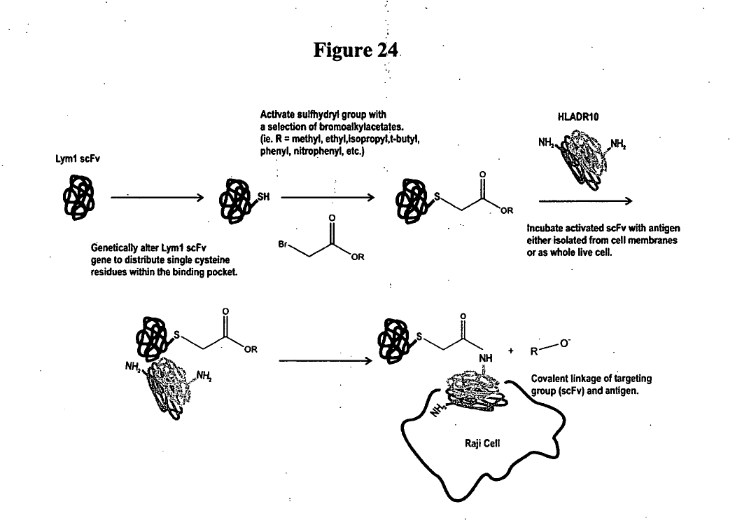

Heterobifunctional cross-linking reagents bearing two electrophiles: a cysteine-specific bromoacetyl group and a lysine-selective carboxylic ester were used. Esters are particularly useful because by changing the leaving group we can tune their reactivity over a broad range. The simplest reagents of this class are bromoacetate esters: when attached to the Cys sulfur (FIG. 24), the reach of the ester is only approximately 2.5 Å. Distances across the protein are an order of magnitude larger, so the effect of moving the site of Cys-mutation dominates the chemical reactivity of the engineered antibody conjugate. Shorter linkers will generally give more selective results in a process that depends on effective local concentration. Placing these short reagents in the binding site of the antibody will limit their accessibility to other macromolecules and serve to reduce their reactivity toward non-binding nucleophiles in solution.

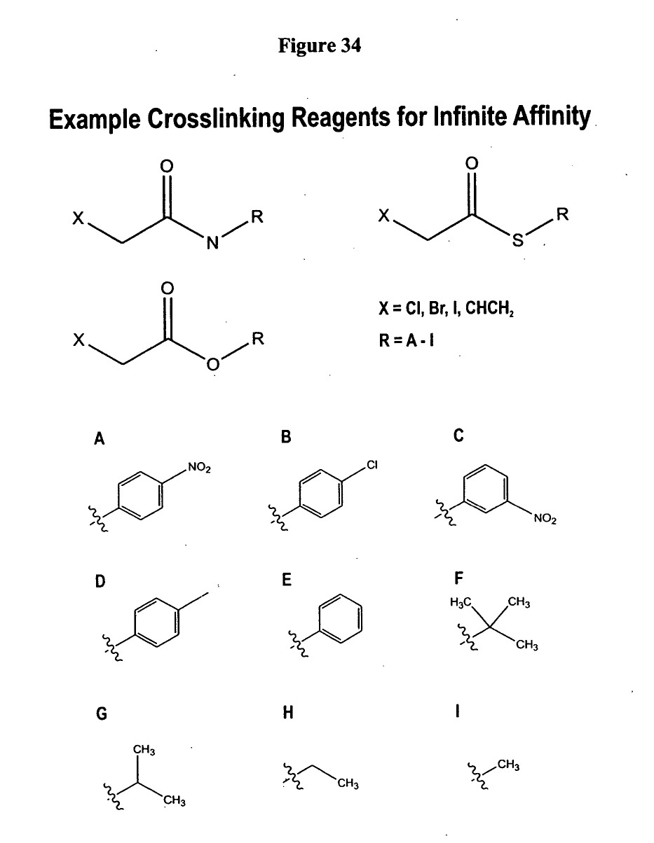

Irreversible Binding Studies of sL1 and HLA-DR10 The general strategy for the development of an irreversibly binding Lym1 sFv (sL1) is outlined in FIG. 24. Lym-1 scFv (sL1) is genetically altered to incorporate a cysteine residue in various positions of one of the six CDRs. This free sulfhydryl is then activated with an alkylbromoacetate consisting of varied R-groups (see FIG. 34) which modify the reactivity of the covalent bond formation. The activated sL1 is then incubated with a crude mixture containing the target protein. The mixture is loaded on a SDS-PAGE gel and then investigated for the appearance of new, high molecular weight bands corresponding to the conjugation of the target protein and the sL1.

The reactive species used in this approach span a range of reactivity that includes predominantly low reactivity species that will not interact irreversibly with non-targeted proteins encountered in the serum and tissues. All linking reagents investigated thus far have been readily available and range in reactivity from very reactive to no expected reactivity.

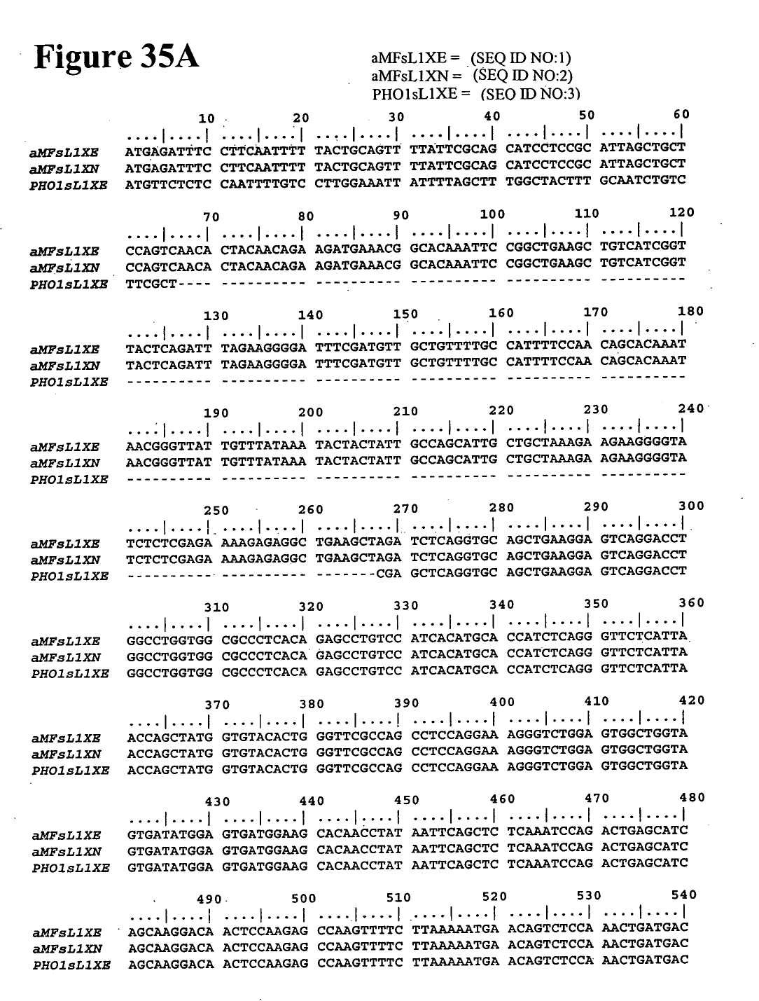

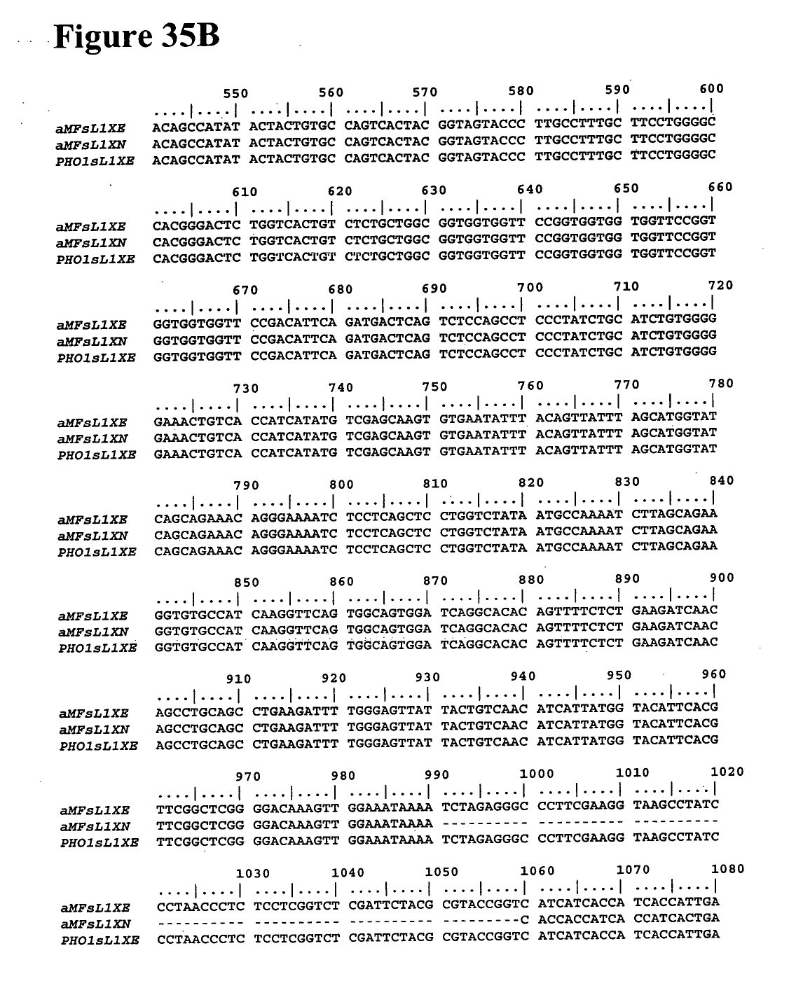

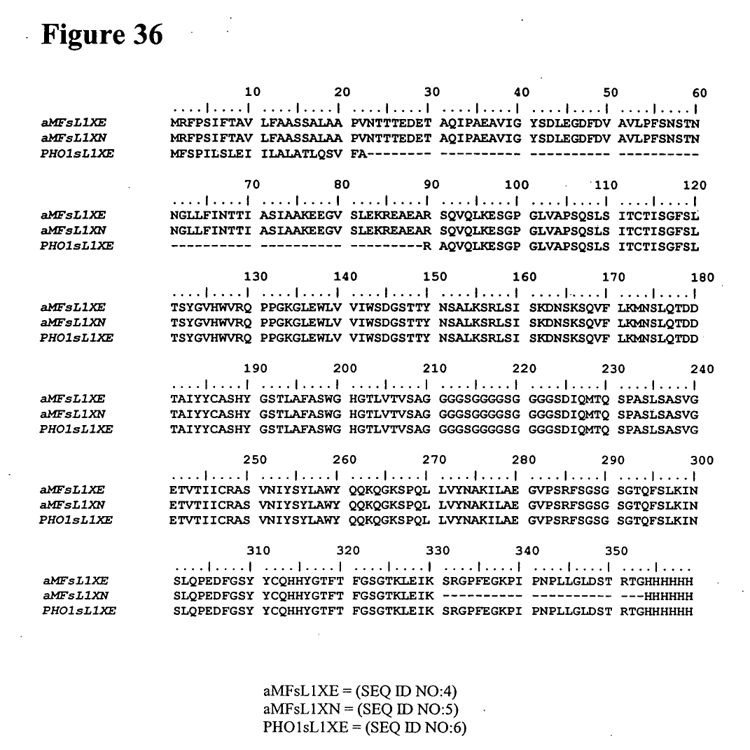

Each single-Cys mutant of sL1 is conjugated with a selection of linkers presently consisting of a set of bromoacetate esters. The addition of a crude preparation of the HLA-DR target antigen permits the association of the sL1 and the antigen through noncovalent interaction. Once associated, the sL1-antigen complex can cross-link for permanent attachment. These molecules (SEQ ID NOs:1-3) are outlined in FIG. 35 below.

The target protein is HLA-DR10, a cell surface protein with two subunits, and a hydrophobic transmembrane anchor. When these subunits are dissociated at 100° C. under reducing conditions, they have an apparent mass on a SDS-PAGE gel of 35 kDa (α subunit) and 31 kDa (β subunit). The Lym-1 single-chain protein fragment has a molecular mass of 29 kDa under similar conditions and thus a sL1-α crosslink will have an molecular mass of 64 kDa. Similarly, a sL1-β subunit crosslink will run at approximately 60 kDa. Due to the fact that an anti-β antibody was used to visualize the following bands, a 57 kDa band is expected for the cross-linked products.

The general experimental protocol for the gel shift assays in the figures is as follows. An aliquot of concentrated P. pastoris expression media containing an sL1 mutant (8-10 μL, positive by anti-V5 stain for sL1 and dialyzed into 10 mM HEPES buffer pH 8.0), is incubated for 15 min with a chosen bromoacetate ester (e.g., nitrophenyl bromoacetate) at a concentration of 10 mM, to alkylate the Cys sidechain. An equimolar amount of sulfhydryl containing small molecule (e.g., cysteine or β-mercaptoethanol) is added to quench unreacted bromoacetate, and a crude HLA-DR10 preparation from Raji cells is added and the mixture incubated at 37° C. for 4 hr. Reducing SAB is added to each sample, followed by SDS-PAGE and western blotting.

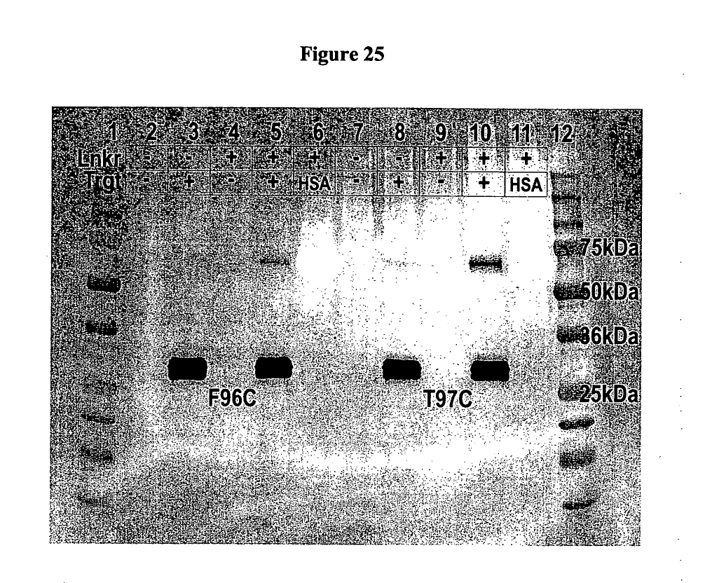

Two different mutants of sL1 that differ by one amino acid in the positioning of the reactive cysteine were compared for reactivity (FIG. 25). The high molecular weight band was suggestive of cross-linking and the obvious intensity difference between mutant F95C and T97C further suggested that the spatial location of the cysteine (and therefore the reactive group) had an influence on the extent of cross-linking.

By staining with the anti-HLA-DR10β antibody HL-40, there is clear evidence that conjugated single-Cys mutants of sL1 bind permanently to their protein targets. FIG. 25 lanes 5 and 10 show two conjugated mutants attaching to the HLA-DR β chain and causing the product to migrate at the position expected. These results show that the sL1 mutant F96C attaches with lower efficiency than the mutant T97C; these two positions are in the light chain CDR3.

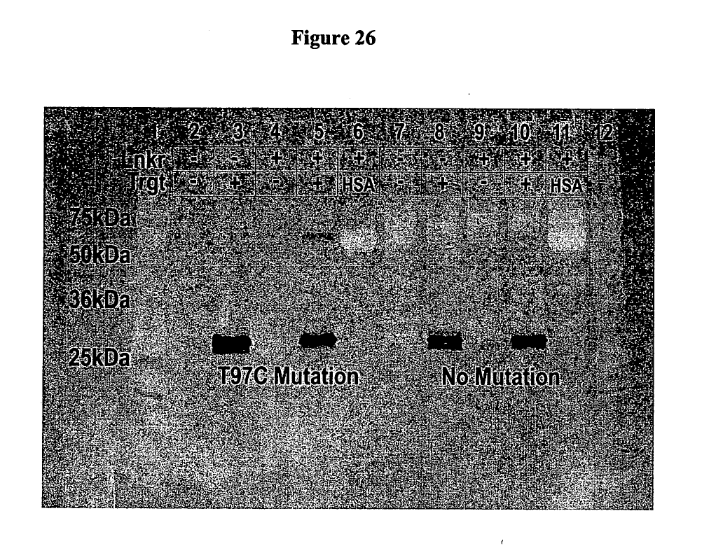

An experiment was conducted that compared the reactivity of the T97C mutant with the reactivity of a wild-type (wt)sL1 that possessed no mutation (FIG. 26). This experiment showed an expected high intensity band for the cysteine mutant and no reactive band appeared for the wt sL1. This strongly indicates a reactivity dependence on the availability of a cysteine to activate for cross-linking.

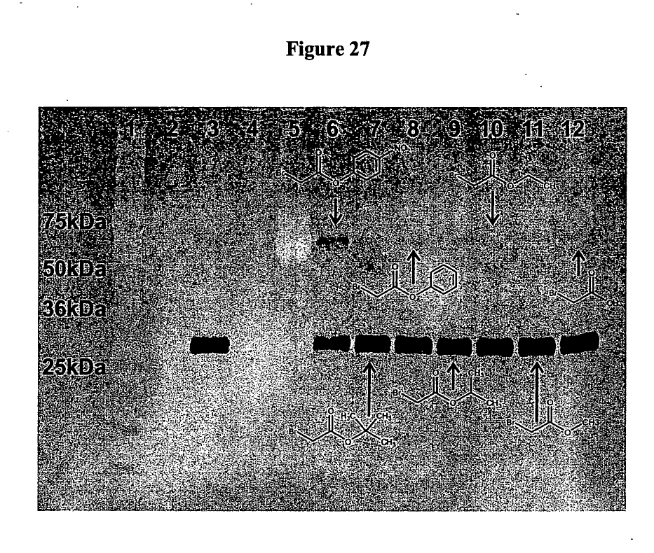

A final experiment was conducted that investigated the reactivity of the linker library (FIG. 27). The T97C mutant was activated with seven different electrophiles shown in FIG. 34 and then allowed to conjugate to HLA-DR10 as in previous experiments. When the mass shift of the target was probed, strong cross-linking was seen for the nitrophenylbromoacetate (FIG. 27, lane 6) followed by mild reactivity of the phenylbromoacete (FIG. 27, lane 8). The assay was insensitive to all remaining electrophiles relative to background. This experiment was suggestive that cross-linking is dependent upon linker reactivity and further suggests that significant work may be required to tune the linker reactivity to an appropriate level for in vivo uses.

Research Design & Methods The technology described above can produce antibody constructs with the appropriate properties to permeate tumors and attach permanently and selectively to cancer cells. We have initially chosen to study anticancer antibodies that bind their targets weakly, because we expect that the strategy of permeating the tumor before permanent attachment will be most easily applied to them. Like the Lym-1 IgG, BC8 IgG has low avidity (K_{D}˜2×10⁻⁸ M); similar to 1F5 (K_{D}˜1−3×10⁻⁸ M); while T84.66 has high avidity (K_{D}˜1×10⁻¹¹ M), providing an interesting example for comparison (lower affinity clones are available if needed). The monovalent binding affinities for scFv's prepared from the genes of these antibodies will be smaller than the avidities by perhaps one to two orders of magnitude, depending on antigen density and accessibility (Adams G. P., et al., Cancer Res. 2001 Jun. 15;61(12):4750-5). We will begin the study of each scFv by measuring their monovalent kₒₙ and k_{off} under comparable conditions at 37° C., to obtain self-consistent monovalent K_{D} values.

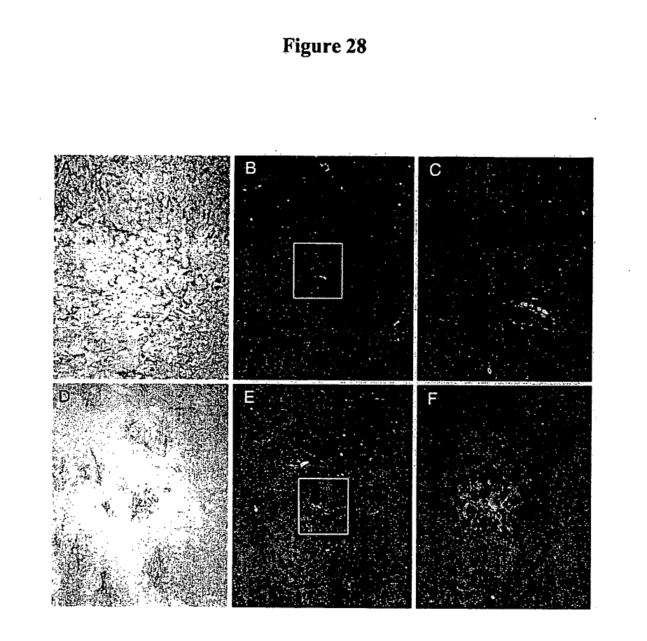

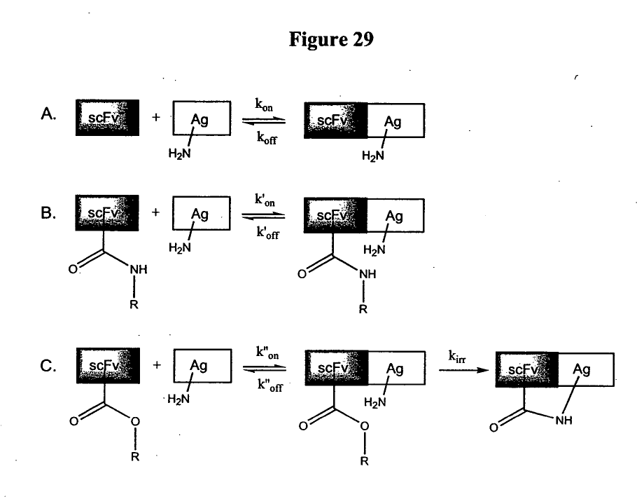

To obtain an scFv distribution like C in FIG. 28, but have it persist with the antigen rather than wash out, we will prepare conjugates with reversibly bound half-lives of the order of a few seconds (which implies a monovalent affinity much weaker than nanomolar for reversible binding: Graff C P, et al. Cancer Res. 2003 Mar. 15;63(6):1288-96), and a low but finite probability of permanent attachment. Developing conjugates suitable for future testing will rely not only on the location of the Cys mutant but also on the reactivity of the appended cross-linker, which may be varied over a considerable range. The simple nitrophenylacetate ester we used in FIG. 27 will probably be the most reactive electrophile employed for this. The probability of permanent attachment will depend on antigen density and tumor physiology, and must ultimately be determined in model systems; we will select conjugates whose attachment efficiencies (=kᵢᵣᵣ/(k″_{off}+kᵢᵣᵣ) in reaction C of FIG. 29) approximately cover the range from 0.1 to as low as we can measure below 0.001.

The effects of mutation and conjugate formation on the rate constants for association and dissociation are likely to vary with the mutation site. Because the mutations are in the CDR's, the on-rate may be decreased in many cases, and this could favor tumor permeation. We will measure kₒₙ and k_{off} for reaction A (FIG. 29) as reference data. We plan to use BIAcore measurements of k′ₒₙ and k′_{off} for control reaction B to see if the steric effect of conjugation is interfering excessively with the association reaction, and to compare the measurements of k″ₒₙ and k″_{off} for reaction C. For k^{irr} we will use in vitro incubations and western blots, since the attachment step is not well suited to measurement with the BIAcore. Targets will be in the form of cultured cells for the in vitro studies, and purified, immobilized antigens for the BIAcore.

Thus, the invention provides a generalized methodology that allows the development of an irreversibly binding antibody or engineered antibody fragment, without prior specific knowledge of antibody or antigen structure or the specific binding orientation.

The embodiment exemplified in this example has specific commercial application, as HLA-DR10 is a target protein overexpressed on the surface of malignant B-cells and may be used to target Non-Hodgkins Lymphoma for therapy or imaging. Similar experiments will be done with the BC8+CD45, IF5+CD20, and T84.66+CEA systems, with the antibodies in monovalent scFv formand any other antibody antigen complex pairs that could prove useful.

### Example 6

### Bispecifc Antibodies

Bispecific antibody constructs. We will design bispecific antitumor+probe capture constructs. Simple scFv proteins are known to quickly penetrate tumors and quickly wash out, clearing to the kidneys (Yokota T, Milenic et al., (1992) Cancer Res. June 15;52(12):3402-8; and Gregory P. Adams, and Robert Schier (1999) Journal of Immunological Methods 231 1999 249-260). Permanently-binding antitumor scFv's will be retained better in tumors and make better images than other scFv's, but due to the short lifetime of scFv's in the circulation (t_{1/2β}≦4 h), tumor uptake will be limited. Preparing diabodies (scFv dimers) doubles the mass of an scFv and provides better binding with two identical binding sites; this leads to substantially better tumor uptake and retention relative to scFv's.

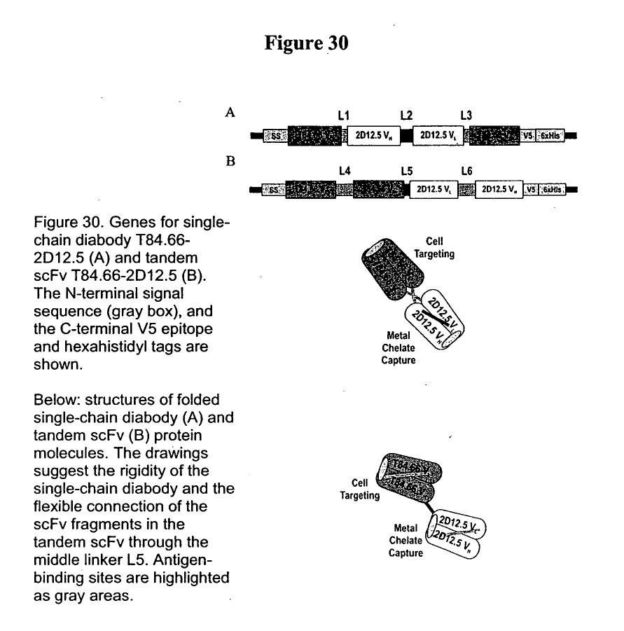

Since permanent binding requires only one binding site, a related single-chain gene may be used to prepare bispecific scFv's incorporating a targeting site for the tumor antigen (HLA-DR, CD45, CD20, CEA) and a capture site for the probe (labeled DOTA or EDTA derivative). This will further improve tumor retention due to permanent binding, and also lead to lower background because of the rapid tumor permeation and kidney elimination of the small DOTA or EDTA probes. FIG. 30 shows the outline for constructing single-chain bispecific scFv's for expression in E. coli and other hosts, however, as will be clear to one of skill in the art any variety of hosts may be employed to express single chain bispecific scFv's.

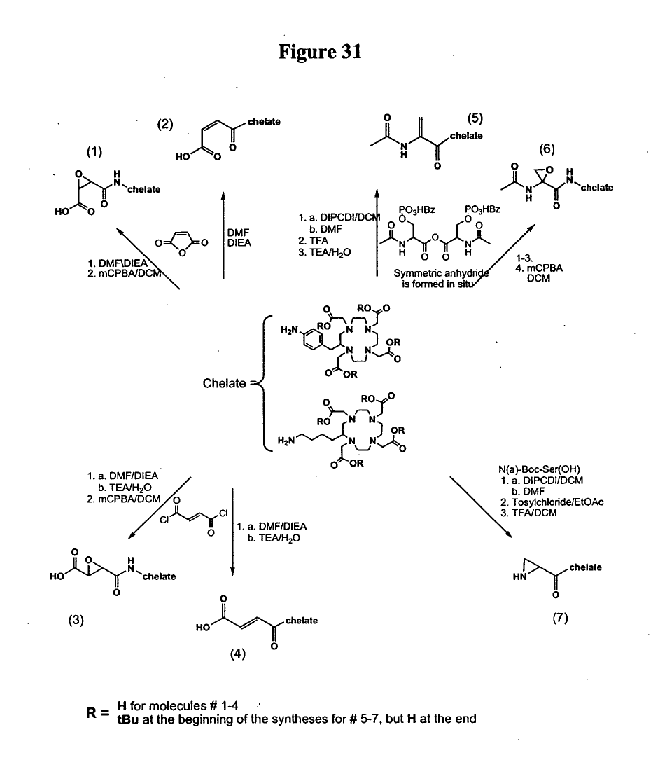

Probes 2 and 4 for 2D12.5 (FIG. 31) have been synthesized. These probes show a range of binding efficiencies, which might be used if a binding site barrier is evident. another approach is to maximize the efficiency of permanent attachment. Different electrophilic DOTA derivatives (FIG. 31), may be tested with 2D12.5 mutants in physiological media in vitro by the methods described herein. Similar chemistry may be used to prepare electrophilic EDTA derivatives for use with CHA255 mutants.

### Example 7

### Lym1 Single Chain Sequence Data

The Pichia pastoris expression system (Invitrogen) was chosen as an expression host for Lym1 single chain antibodies. Initially, 3 expression constructs (αMFsL1XE, αMFsL1XN, and PHO1sL1XE) were prepared as outlined in FIG. 35 (DNA) (SEQ ID NOs: 1-3), FIG. 36 (Protein) (SEQ ID NOs: 4-6), and Table 4 (Features).

TABLE 4
Feature list for αMFsL1XE

| Base Pairs | Feature | aMFsL1XE | aMFsL1XN | PHO1sL1XE |
|---|---|---|---|---|
| 1-267 | αMF Secretion | Present | Present | Uses PHO1 Secretion Signal |
| | Signal | | | |
| 268-273 | BglII | Present | Present | AGATCT to CGAGCT |
| | | | | coding removed restriction |
| | | | | site |
| 274-990 | Lym 1 Single Chain | Present | Present | Present |
| | Gene | | | |
| 991-1008 | XbaI, ApaI, BstBI | Present | Not Present | Present |
| 1009-1050 | V5 Epitope | Present | Not Present | Present |
| 1051-1059 | AgeI | Present | Not Present | Present |
| 1060-1077 | 6xHis Epitope | Present | Present | Present |
| 1078-1080 | Stop Codon | Present | Present | Present |

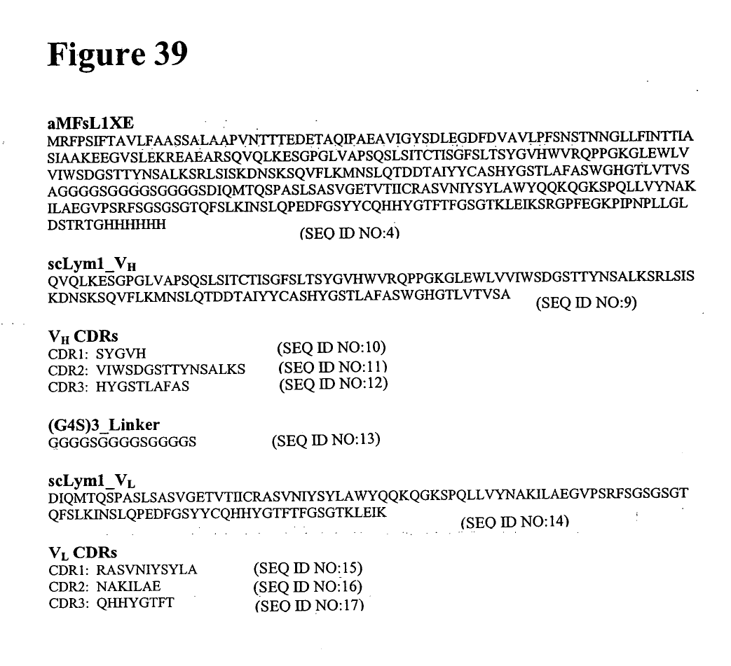

Note:
Basepair numbering in Table 1 applies to aMFsL1XE DNA sequence (SEQ ID NO: 4) only (FIG. 39). Feature changes for aMFsL1XN and PHO1sL1XE cause numbering differences and are included for comparison only.

The αNMFsL1XE and αMFsL1XN expression cassettes both used the commercial alpha Mating-Factor (αMF) secretion signal as provided by Invitrogen and a C-terminal 6xHis epitope tag for purification and affinity staining. These cassettes differed in that αMFsL1XE also included a V5 epitope tag between the C-terminus of the Lym 1 single-chain (sL1) coding region and the 6×His epitope. The third construct, PHO1sL1XE, uses the acid phosphatase (PHO1) secretion signal and included a V5 and 6×His C-terminal epitope.

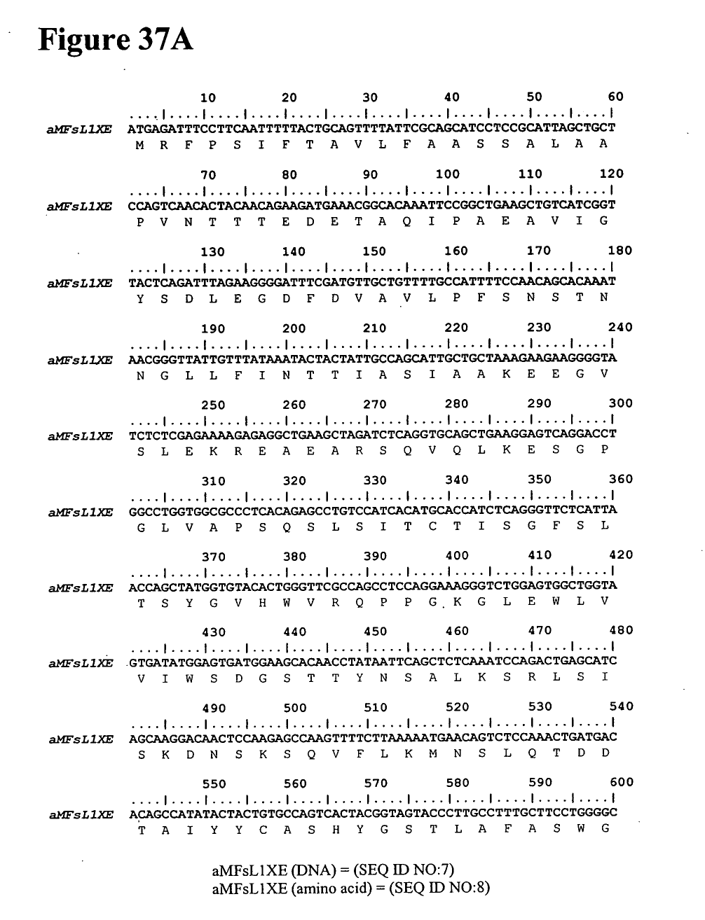

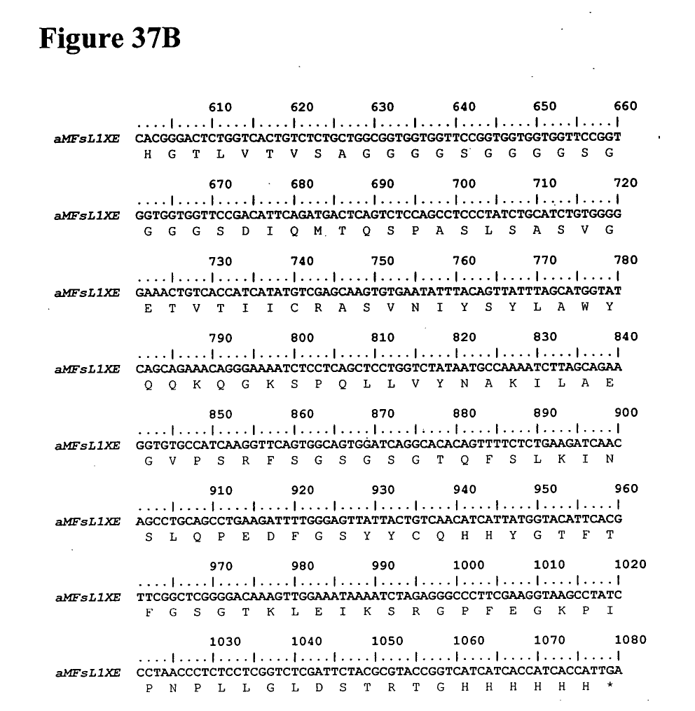

There was no remarkable difference between secretion levels of sL1 using the αMF of PHO1 secretion signals. Thus, the αMF secretion signal was selected due to the greater body of literature citing successes. The αMFsL1XE expression cassette was used as a genetic template for the construction of Lym 1 single chain library and a translated sequence alignment is presented in FIG. 37 (SEQ ID NOs: 7-8).

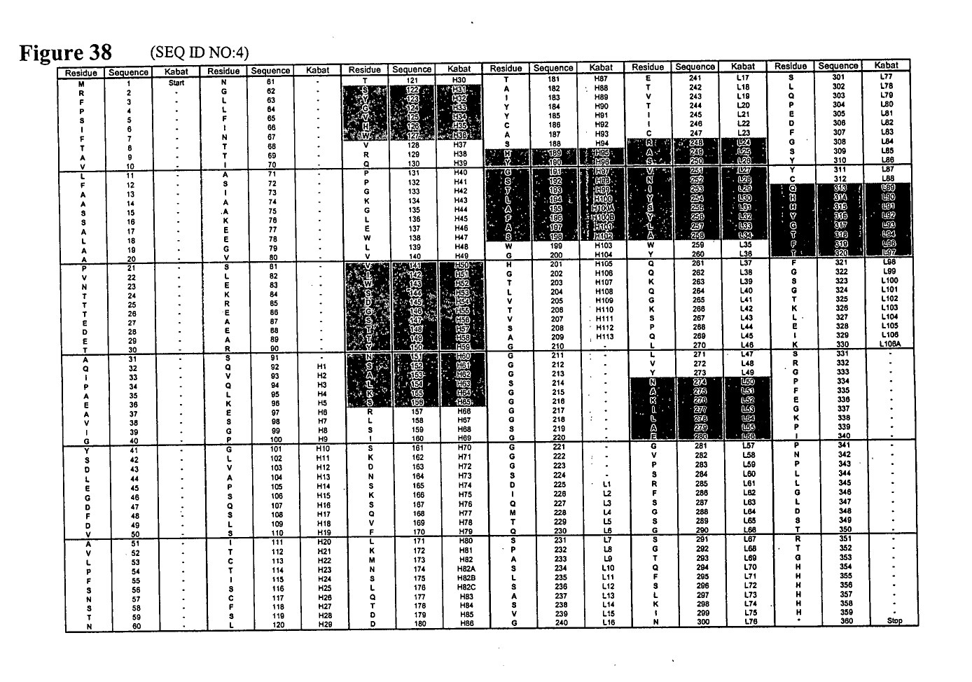

Complementarity Determining Regions (CDRs) were selected based on Kabat definitions and formed the basis for the selection of residues to ‘scan’ with a cysteine replacement. The expression cassette αMFsL1XE is shown in FIG. 38 (SEQ ID NO:4) with sequential numbering alignment and Kabat numbering scheme for the variable regions of sL1 (both heavy and light). FIG. 39 depicts selected sequence features of αMFsL1XE (SEQ ID NO:4), namely the CDRs (SEQ ID NOs: 10-12, 15-17), sL1 heavy (SEQ ID NO:9) and light chain (SEQ ID NO:14) coding regions, and the artificial connecting linker (SEQ ID NO:13).

The definition of the CDR region is based on Kabat CDR definitions which have their basis in sequence homology modeling. This is because few antibody crystal structures were determined at the time the system was proposed. In the 20 years since then, a number of different methods for CDR determination have surfaced which generally agree on some definitions (such as the variable light chain CDRs) and are significantly different on other definitions (such as the variable heavy, in particular the CDR-H3). Table 5 outlines how some of these different methods affect the CDR definitions for sL1.

TABLE 5
Varied CDR Definitions

| | | | |
|---|---|---|---|
| Kabat | AbM | Chothia | Contact |

| | | | | |
|---|---|---|---|---|
| V_{H}CDRI | (SEQ ID NO:10) | (SEQ ID NO:28) | (SEQ ID NO:30) | (SEQ ID NO:32) |
| | SYGVH | GFSLTSYGVH | GFSLTSY | TSYGVH |
| V_{H}CDR2 | (SEQ ID NO:11) | (SEQ ID NO:29) | (SEQ ID NO:31) | (SEQ ID NO:33) |
| | VIWSDGSTTYNSALKS | VIWSDGSTT | WSDGS | WLVVIWSDGSTT |
| V_{H}CDR3 | (SEQ ID NO:12) | SEQ ID NO:12) | (SEQ ID NO:12) | (SEQ ID NO:34) |
| | HYGSTLAFAS | HYGSTLAFAS | HYGSTLAFAS | ASHYGSTLAFA |
| V_{L}CDR1 | (SEQ ID NO:15) | (SEQ ID NO:15) | (SEQ ID NO:15) | (SEQ ID NO:35) |
| | RASVNIYSYLA | RASVNIYSYLA | RASVNIYSYLA | YSYLAWY |
| V_{L}CDR2 | (SEQ ID NO:16) | (SEQ ID NO:16) | (SEQ ID NO:16) | (SEQ ID NO:36) |
| | NAKILAE | NAKILAE | NAKILAE | LLVYNAKILA |
| V_{L}CDR3 | (SEQ ID NO:17) | (SEQ ID NO:17) | (SEQ ID NO:17) | (SEQ ID NO:37) |
| | QHHYGTFT | QHHYGTFT | QHHYGTFT | QHHYGTF |

Larger bold font indicates an additional residue relative to Kabat CDR definition. The Kabat definitions, being the most widely accepted and referenced scheme, was used as the basis for our CDR definitions of sL 1.

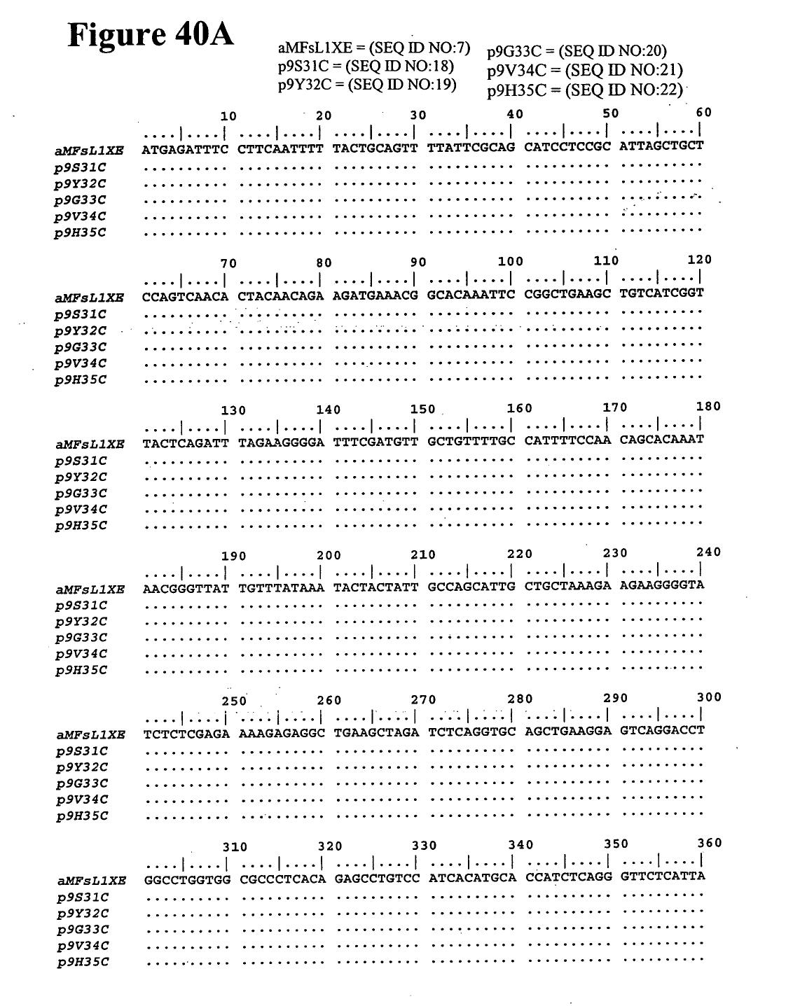

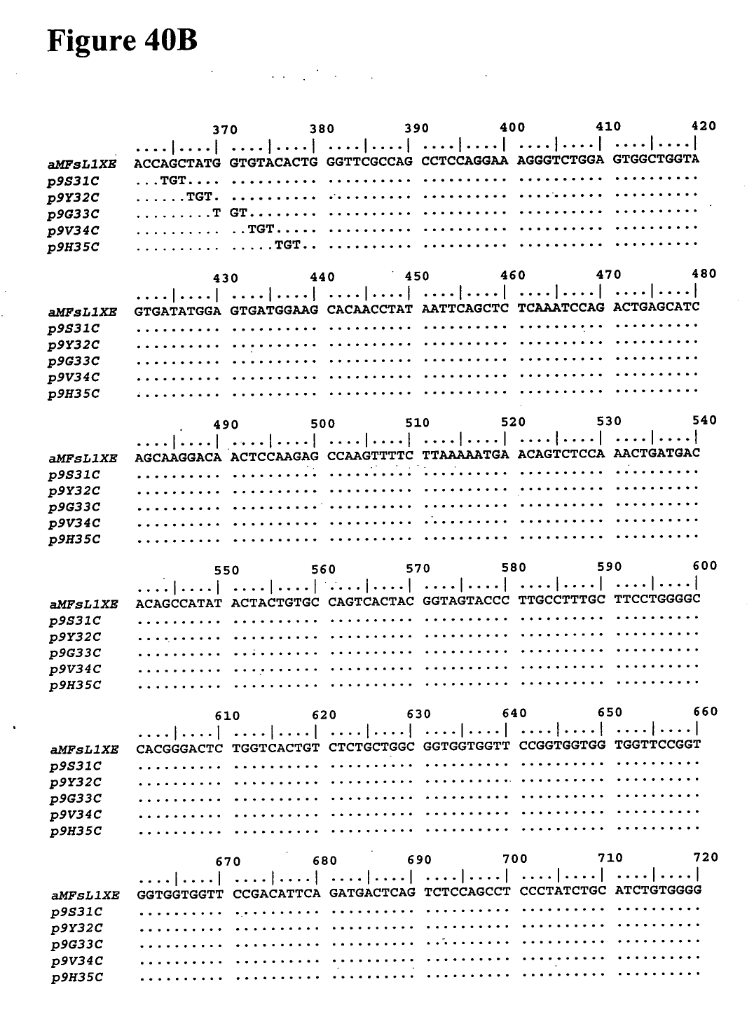

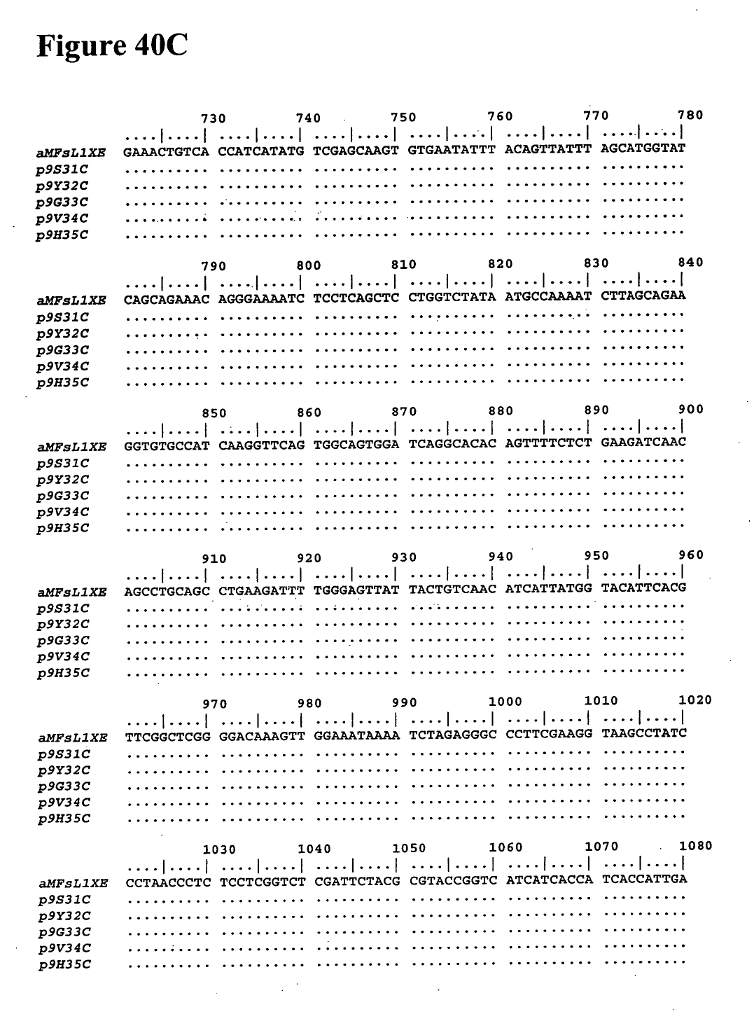

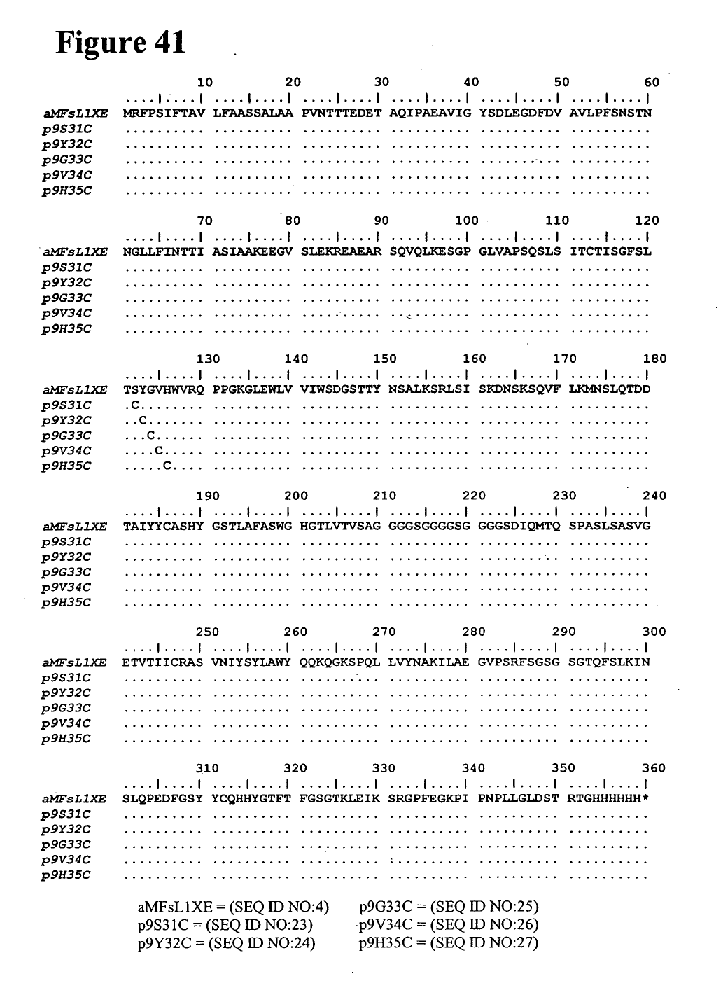

Following determination of the CDR regions of sL1, site-directed mutagenesis was used to change the native DNA coding sequence for each CDR amino acid to DNA coding for cysteine (TGT/TGC) in the translated protein sequence (FIGS. 40 and 41) (SEQ ID NOs:18-22, 23-27). These new genes with single cysteine mutations were investigated individually.

It is understood that the examples and embodiments described herein are for illustrative purposes only and that various modifications or changes in light thereof will be suggested to persons skilled in the art and are to included within the spirit and purview of this application and are considered within the scope of the appended claims. All publications, patents, and patent applications cited herein are hereby incorporated by reference in their entirety for all purposes.

What is claimed is:

1. A mutant antibody comprising:
(i) a mutant polypeptide sequence, comprising a mutant amino acid at a position within or proximate to a complimentarity determining region of said antibody, wherein said mutant amino acid is not present at said position in the wild type of said antibody; and
(ii) a linker covalently bound to said mutant amino acid, said linker comprising a reactive functional group.

2. The mutant antibody according to claim 1, wherein said linker is a member selected from the group consisting of substituted or unsubstituted alkyl, substituted or unsubstituted heteroalkyl, substituted or unsubstituted aryl, substituted or unsubstituted heteroaryl, substituted or unsubstituted heterocycloalkyl moieties.

3. The mutant antibody according to claim 1, wherein said antibody comprises a first domain that specifically binds to a cell surface antigen.

4. The antibody according to claim 3, wherein said antibody comprises a second domain that specifically binds a metal chelate.

5. An anitibody-antigen complex formed between an antibody of claim 1 and an antigen to which said antibody specifically binds.

6. The complex according to claim 5, wherein said reactive functional group is converted to a covalent bond by reaction with a group of complementary reactivity on said antigen, linking said antibody and said antigen through said linker.

7. A method of forming an antibody antigen complex that does not dissociate under physiologically relevant conditions, said method comprising:
(a) contacting said antigen with a mutant antibody comprising:
(i) a mutant polypeptide sequence, comprising a mutant amino acid at a position within or proximate to a complementarity determining region of said antibody, wherein said mutant amino acid is not present at said position in the wild type of said antibody; and
(ii) a linker covalently bound to said mutant amino acid, said linker comprising a reactive functional group, under conditions appropriate to complex said antibody to said antigen; and
(b) forming a covalent bond between said reactive functional group and a group of complementary reactivity on said antigen, thereby forming said antigen-antibody complex.
